SEQUENCE LISTING (219 sequences)

SEQ ID NO: 1 - DNA, 19 bp, Synthetic construct
  source 1..19: mol_type = other DNA
ggtctcctccatcagcacc

SEQ ID NO: 2 - DNA, 19 bp, synthetic construct
  misc_feature 1..3: note = RNA
  source 1..19: mol_type = other DNA
  misc_feature 4..16: note = DNA
  misc_feature 17..19: note = RNA
  modified_base 3: mod_base = OTHER; note = uracil
  modified_base 1..2: mod_base = OTHER; note = 2 prime OME phosphorothioate
  modified_base 17..19: mod_base = OTHER; note = 2 prime OME phosphorothioate
ggtctcctccatcagcacc

SEQ ID NO: 3 - DNA, 19 bp, synthetic construct
  misc_feature 1..6: note = RNA
  source 1..19: mol_type = other DNA
  misc_feature 7..19: note = DNA
  modified_base 3: mod_base = OTHER; note = uracil
  modified_base 5: mod_base = OTHER; note = uracil
  modified_base 1..2: mod_base = OTHER; note = 2 prime OME phosphorothioate
  modified_base 4: mod_base = OTHER; note = 2 prime OME phosphorothioate
  modified_base 6: mod_base = OTHER; note = 2 prime OME phosphorothioate
ggtctcctccatcagcacc

SEQ ID NO: 4 - DNA, 19 bp, Synthetic construct
  source 1..19: mol_type = other DNA
ggtctcctccatcagcacc

SEQ ID NO: 5 - DNA, 19 bp, Synthetic construct
  source 1..19: mol_type = other DNA
ggtctcctccatcagcacc

SEQ ID NO: 6 - DNA, 19 bp, Synthetic construct
  source 1..19: mol_type = other DNA
ggtctcctccatcagcacc

SEQ ID NO: 7 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 10: mod_base = OTHER; note = uracil
  modified_base 15..16: mod_base = OTHER; note = uracil
  modified_base 11..14: mod_base = OTHER; note = 2 prime OME nucleotide
ggccaaacctcggcttacct

SEQ ID NO: 8 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 10: mod_base = OTHER; note = uracil
  modified_base 11..14: mod_base = OTHER; note = 2 prime OME nucleotide
  modified_base 15..16: mod_base = OTHER; note = uracil
ggccaaacctcggcttacct

SEQ ID NO: 9 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 10: mod_base = OTHER; note = uracil
  modified_base 11..14: mod_base = OTHER; note = 2 prime OME nucleotide
  modified_base 15..16: mod_base = OTHER; note = uracil
ggccaaacctcggcttacct

SEQ ID NO: 10 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 10: mod_base = OTHER; note = uracil
  modified_base 15..16: mod_base = OTHER; note = uracil
  modified_base 11..14: mod_base = OTHER; note = 2 prime OME nucleotide
ggccaaacctcggcttacct

SEQ ID NO: 11 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 10: mod_base = OTHER; note = uracil
  modified_base 15..16: mod_base = OTHER; note = uracil
  modified_base 11..14: mod_base = OTHER; note = 2 prime OME nucleotide
ggccaaacctcggcttacct

SEQ ID NO: 12 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 10: mod_base = OTHER; note = uracil
  modified_base 15..16: mod_base = OTHER; note = uracil
  modified_base 11..14: mod_base = OTHER; note = 2 prime OME nucleotide
ggccaaacctcggcttacct

SEQ ID NO: 13 - DNA, 20 bp, synthetic construct
  misc_feature 1..3: note = DNA
  source 1..20: mol_type = other DNA
  misc_feature 4..20: note = RNA
  modified_base 4..9: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
  modified_base 11..14: mod_base = OTHER; note = 2 prime OME nucleotide
  modified_base 17..19: mod_base = OTHER; note = 2 prime OME nucleotide
ggccaaacctcggcttacct

SEQ ID NO: 14 - DNA, 20 bp, synthetic construct
  misc_feature 1..4: note = RNA
  source 1..20: mol_type = other DNA
  misc_feature 5..7: note = DNA
  misc_feature 8..20: note = RNA
  modified_base 8..9: mod_base = OTHER; note = 2 prime OME nucleotide
  modified_base 11..14: mod_base = OTHER; note = 2 prime OME nucleotide
  modified_base 1..4: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 15 - DNA, 20 bp, synthetic construct
  misc_feature 1..8: note = RNA
  source 1..20: mol_type = other DNA
  misc_feature 9..11: note = DNA
  misc_feature 12..20: note = RNA
  modified_base 1..8: mod_base = OTHER; note = 2 prime OME nucleotide
  modified_base 12..14: mod_base = OTHER; note = 2 prime OME nucleotide
  modified_base 15..16: mod_base = OTHER; note = uracil
  modified_base 20: mod_base = OTHER; note = uracil
  modified_base 17..19: mod_base = OTHER; note = 2 prime OME nucleotide
ggccaaacctcggcttacct

SEQ ID NO: 16 - DNA, 20 bp, synthetic construct
  misc_feature 1..12: note = RNA
  source 1..20: mol_type = other DNA
  misc_feature 13..15: note = DNA
  misc_feature 16..20: note = RNA
  modified_base 1..9: mod_base = OTHER; note = 2 prime OME nucleotide
  modified_base 10: mod_base = OTHER; note = uracil
  modified_base 16: mod_base = OTHER; note = uracil
  modified_base 20: mod_base = OTHER; note = uracil
  modified_base 11..12: mod_base = OTHER; note = 2 prime OME nucleotide
  modified_base 17..19: mod_base = OTHER; note = 2 prime OME nucleotide
ggccaaacctcggcttacct

SEQ ID NO: 17 - DNA, 20 bp, synthetic construct
  misc_feature 1..3: note = DNA
  source 1..20: mol_type = other DNA
  misc_feature 4..16: note = RNA
  misc_feature 17..18: note = DNA
  misc_feature 19..20: note = RNA
  modified_base 4..9: mod_base = OTHER; note = 2 prime OME nucleotide
  modified_base 19: mod_base = OTHER; note = 2 prime OME nucleotide
  modified_base 11..14: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 18 - DNA, 20 bp, synthetic construct
  misc_feature 1..17: note = RNA
  source 1..20: mol_type = other DNA
  misc_feature 18..20: note = DNA
  modified_base 1..9: mod_base = OTHER; note = 2 prime OME nucleotide
  modified_base 11..14: mod_base = OTHER; note = 2 prime OME nucleotide
  modified_base 17: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 19 - DNA, 20 bp, synthetic construct
  misc_feature 1..4: note = DNA
  source 1..20: mol_type = other DNA
  misc_feature 5..20: note = RNA
  modified_base 5..9: mod_base = OTHER; note = 2 prime OME nucleotide
  modified_base 11..14: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 20 - DNA, 20 bp, synthetic construct
  misc_feature 1..4: note = RNA
  source 1..20: mol_type = other DNA
  misc_feature 5..8: note = DNA
  misc_feature 9..20: note = RNA
  modified_base 1..4: mod_base = OTHER; note = 2 prime OME nucleotide
  modified_base 9: mod_base = OTHER; note = 2 prime OME nucleotide
  modified_base 11..14: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 21 - DNA, 20 bp, synthetic construct
  misc_feature 1..8: note = RNA
  source 1..20: mol_type = other DNA
  misc_feature 9..12: note = DNA
  misc_feature 13..20: note = RNA
  modified_base 1..8: mod_base = OTHER; note = 2 prime OME nucleotide
  modified_base 13..14: mod_base = OTHER; note = 2 prime OME nucleotide
  modified_base 17..19: mod_base = OTHER; note = 2 prime OME nucleotide
  modified_base 15..16: mod_base = OTHER; note = uracil
  modified_base 20: mod_base = OTHER; note = uracil
ggccaaacctcggcttacct

SEQ ID NO: 22 - DNA, 20 bp, synthetic construct
  misc_feature 1..12: note = RNA
  source 1..20: mol_type = other DNA
  misc_feature 13..16: note = DNA
  misc_feature 17..20: note = RNA
  modified_base 1..9: mod_base = OTHER; note = 2 prime OME nucleotide
  modified_base 11..12: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 20: mod_base = OTHER; note = uracil
ggccaaacctcggcttacct

SEQ ID NO: 23 - DNA, 20 bp, synthetic construct
  misc_feature 1..13: note = RNA
  source 1..20: mol_type = other DNA
  misc_feature 14..20: note = DNA
  modified_base 1..9: mod_base = OTHER; note = 2 prime OME nucleotide
  modified_base 11..13: mod_base = OTHER; note = 2 prime OME nucleotide
  modified_base 10: mod_base = OTHER; note = uracil
ggccaaacctcggcttacct

SEQ ID NO: 24 - DNA, 20 bp, synthetic construct
  misc_feature 1..14: note = RNA
  source 1..20: mol_type = other DNA
  misc_feature 15..20: note = DNA
  modified_base 1..9: mod_base = OTHER; note = 2 prime OME nucleotide
  modified_base 11..14: mod_base = OTHER; note = 2 prime OME nucleotide
  modified_base 10: mod_base = OTHER; note = uracil
ggccaaacctcggcttacct

SEQ ID NO: 25 - DNA, 20 bp, synthetic construct
  misc_feature 1..15: note = RNA
  source 1..20: mol_type = other DNA
  misc_feature 16..20: note = DNA
  modified_base 1..9: mod_base = OTHER; note = 2 prime OME nucleotide
  modified_base 11..14: mod_base = OTHER; note = 2 prime OME nucleotide
  modified_base 10: mod_base = OTHER; note = uracil
  modified_base 15: mod_base = OTHER; note = uracil
ggccaaacctcggcttacct

SEQ ID NO: 26 - DNA, 20 bp, synthetic construct
  misc_feature 1..17: note = RNA
  source 1..20: mol_type = other DNA
  misc_feature 18..20: note = DNA
  modified_base 1..9: mod_base = OTHER; note = 2 prime OME nucleotide
  modified_base 11..14: mod_base = OTHER; note = 2 prime OME nucleotide
  modified_base 17: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 27 - DNA, 20 bp, synthetic construct
  misc_feature 1..18: note = RNA
  source 1..20: mol_type = other DNA
  misc_feature 19..20: note = DNA
  modified_base 1..9: mod_base = OTHER; note = 2 prime OME nucleotide
  modified_base 11..14: mod_base = OTHER; note = 2 prime OME nucleotide
  modified_base 17..18: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 28 - DNA, 20 bp, synthetic construct
  misc_feature 1..3: note = DNA
  source 1..20: mol_type = other DNA
  misc_feature 4..20: note = RNA
  modified_base 4..9: mod_base = OTHER; note = 2 prime OME nucleotide
  modified_base 11..14: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 29 - DNA, 20 bp, synthetic construct
  misc_feature 1..4: note = RNA
  source 1..20: mol_type = other DNA
  misc_feature 5..7: note = DNA
  misc_feature 8..20: note = RNA
  modified_base 1..4: mod_base = OTHER; note = 2 prime OME nucleotide
  modified_base 8..9: mod_base = OTHER; note = 2 prime OME nucleotide
  modified_base 11..14: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 30 - DNA, 20 bp, synthetic construct
  misc_feature 1..8: note = RNA
  source 1..20: mol_type = other DNA
  misc_feature 9..11: note = DNA
  misc_feature 12..20: note = RNA
  modified_base 1..8: mod_base = OTHER; note = 2 prime OME nucleotide
  modified_base 12..14: mod_base = OTHER; note = 2 prime OME nucleotide
  modified_base 17..19: mod_base = OTHER; note = 2 prime OME nucleotide
  modified_base 15..16: mod_base = OTHER; note = uracil
  modified_base 20: mod_base = OTHER; note = uracil
ggccaaacctcggcttacct

SEQ ID NO: 31 - DNA, 20 bp, synthetic construct
  misc_feature 1..12: note = RNA
  source 1..20: mol_type = other DNA
  misc_feature 13..15: note = DNA
  misc_feature 16..20: note = RNA
  modified_base 1..9: mod_base = OTHER; note = 2 prime OME nucleotide
  modified_base 11..12: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 16: mod_base = OTHER; note = uracil
  modified_base 20: mod_base = OTHER; note = uracil
ggccaaacctcggcttacct

SEQ ID NO: 32 - DNA, 20 bp, synthetic construct
  misc_feature 1..3: note = DNA
  source 1..20: mol_type = other DNA
  misc_feature 4..16: note = RNA
  misc_feature 17..18: note = DNA
  misc_feature 19..20: note = RNA
  modified_base 4..9: mod_base = OTHER; note = 2 prime OME nucleotide
  modified_base 11..14: mod_base = OTHER; note = 2 prime OME nucleotide
  modified_base 19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 33 - DNA, 20 bp, synthetic construct
  misc_feature 1..17: note = RNA
  source 1..20: mol_type = other DNA
  misc_feature 18..20: note = DNA
  modified_base 1..9: mod_base = OTHER; note = 2 prime OME nucleotide
  modified_base 11..14: mod_base = OTHER; note = 2 prime OME nucleotide
  modified_base 17: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 34 - DNA, 20 bp, synthetic construct
  misc_feature 1..4: note = DNA
  source 1..20: mol_type = other DNA
  misc_feature 5..20: note = RNA
  modified_base 5..9: mod_base = OTHER; note = 2 prime OME nucleotide
  modified_base 11..14: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 35 - DNA, 20 bp, synthetic construct
  misc_feature 1..4: note = RNA
  source 1..20: mol_type = other DNA
  misc_feature 5..8: note = DNA
  misc_feature 9..20: note = RNA
  modified_base 1..4: mod_base = OTHER; note = 2 prime OME nucleotide
  modified_base 9: mod_base = OTHER; note = 2 prime OME nucleotide
  modified_base 11..14: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 15..16: mod_base = OTHER; note = uracil
  modified_base 20: mod_base = OTHER; note = uracil
ggccaaacctcggcttacct

SEQ ID NO: 36 - DNA, 20 bp, synthetic construct
  misc_feature 1..8: note = RNA
  source 1..20: mol_type = other DNA
  misc_feature 9..12: note = DNA
  misc_feature 13..20: note = RNA
  modified_base 1..8: mod_base = OTHER; note = 2 prime OME nucleotide
  modified_base 13..14: mod_base = OTHER; note = 2 prime OME nucleotide
  modified_base 17..19: mod_base = OTHER; note = 2 prime OME nucleotide
  modified_base 15..16: mod_base = OTHER; note = uracil
  modified_base 20: mod_base = OTHER; note = uracil
ggccaaacctcggcttacct

SEQ ID NO: 37 - DNA, 20 bp, synthetic construct
  misc_feature 1..12: note = RNA
  source 1..20: mol_type = other DNA
  misc_feature 13..16: note = DNA
  misc_feature 17..20: note = RNA
  modified_base 1..9: mod_base = OTHER; note = 2 prime OME nucleotide
  modified_base 11..12: mod_base = OTHER; note = 2 prime OME nucleotide
  modified_base 17..19: mod_base = OTHER; note = 2 prime OME nucleotide
  modified_base 10: mod_base = OTHER; note = uracil
  modified_base 20: mod_base = OTHER; note = uracil
ggccaaacctcggcttacct

SEQ ID NO: 38 - DNA, 20 bp, synthetic construct
  misc_feature 1..13: note = RNA
  source 1..20: mol_type = other DNA
  misc_feature 14..20: note = DNA
  modified_base 1..9: mod_base = OTHER; note = 2 prime OME nucleotide
  modified_base 11..13: mod_base = OTHER; note = 2 prime OME nucleotide
  modified_base 10: mod_base = OTHER; note = uracil
ggccaaacctcggcttacct

SEQ ID NO: 39 - DNA, 20 bp, synthetic construct
  misc_feature 1..14: note = RNA
  source 1..20: mol_type = other DNA
  misc_feature 15..20: note = DNA
  modified_base 1..9: mod_base = OTHER; note = 2 prime OME nucleotide
  modified_base 11..14: mod_base = OTHER; note = 2 prime OME nucleotide
  modified_base 10: mod_base = OTHER; note = uracil
ggccaaacctcggcttacct

SEQ ID NO: 40 - DNA, 20 bp, synthetic construct
  misc_feature 1..15: note = RNA
  source 1..20: mol_type = other DNA
  misc_feature 16..20: note = DNA
  modified_base 1..9: mod_base = OTHER; note = 2 prime OME nucleotide
  modified_base 11..14: mod_base = OTHER; note = 2 prime OME nucleotide
  modified_base 10: mod_base = OTHER; note = uracil
  modified_base 15: mod_base = OTHER; note = uracil
ggccaaacctcggcttacct

SEQ ID NO: 41 - DNA, 20 bp, synthetic construct
  misc_feature 1..17: note = RNA
  source 1..20: mol_type = other DNA
  misc_feature 18..20: note = DNA
  modified_base 1..9: mod_base = OTHER; note = 2 prime OME nucleotide
  modified_base 11..14: mod_base = OTHER; note = 2 prime OME nucleotide
  modified_base 17: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 42 - DNA, 20 bp, synthetic construct
  misc_feature 1..18: note = RNA
  source 1..20: mol_type = other DNA
  misc_feature 19..20: note = DNA
  modified_base 1..9: mod_base = OTHER; note = 2 prime OME nucleotide
  modified_base 11..14: mod_base = OTHER; note = 2 prime OME nucleotide
  modified_base 17..18: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 43 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 11..14: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 44 - DNA, 20 bp, synthetic construct
  misc_feature 1..16: note = RNA
  source 1..20: mol_type = other DNA
  misc_feature 17..20: note = DNA
  modified_base 1..9: mod_base = OTHER; note = 2 prime OME nucleotide
  modified_base 11..14: mod_base = OTHER; note = 2 prime OME nucleotide
  modified_base 10: mod_base = OTHER; note = uracil
  modified_base 15..16: mod_base = OTHER; note = uracil
ggccaaacctcggcttacct

SEQ ID NO: 45 - DNA, 25 bp, Synthetic construct
  source 1..25: mol_type = other DNA
tcgtaacgttacgtattcgaatcgt

SEQ ID NO: 46 - DNA, 25 bp, Synthetic construct
  source 1..25: mol_type = other DNA
tcgtaacgttacgtattcgaatcgt

SEQ ID NO: 47 - DNA, 25 bp, Synthetic construct
  source 1..25: mol_type = other DNA
acgattcgaatacgtaacgttacga

SEQ ID NO: 48 - DNA, 25 bp, Synthetic construct
  source 1..25: mol_type = other DNA
acgattcgaatacgtaacgttacga

SEQ ID NO: 49 - DNA, 25 bp, Synthetic construct
  source 1..25: mol_type = other DNA
tcgtaacgttacgtattcgaatcgt

SEQ ID NO: 50 - DNA, 25 bp, Synthetic construct
  source 1..25: mol_type = other DNA
tcgtaacgttacgtattcgaatcgt

SEQ ID NO: 51 - DNA, 25 bp, Synthetic construct
  source 1..25: mol_type = other DNA
acgattcgaatacgtaacgttacga

SEQ ID NO: 52 - DNA, 25 bp, Synthetic construct
  source 1..25: mol_type = other DNA
acgattcgaatacgtaacgttacga

SEQ ID NO: 53 - DNA, 25 bp, Synthetic construct
  source 1..25: mol_type = other DNA
tcgtaacgttacgtattcgaatcgt

SEQ ID NO: 54 - DNA, 25 bp, Synthetic construct
  source 1..25: mol_type = other DNA
acgattcgaatacgtaacgttacga

SEQ ID NO: 55 - DNA, 25 bp, Synthetic construct
  source 1..25: mol_type = other DNA
acgattcgaatacgtaacgttacga

SEQ ID NO: 56 - DNA, 25 bp, Synthetic construct
  source 1..25: mol_type = other DNA
tcgtaacgttacgtattcgaatcgt

SEQ ID NO: 57 - DNA, 3512 bp, Mus musculus
  source 1..3512: mol_type = other DNA
tggggattaagaggggggaatgtaacaggtcccgtttgcagcccaattaggatttggggttttgtcctcactctgagcgtcatttgacgctgtctggggagggcgaggccgaaacctgatcctttagtaccggggcccgttaatgtttaatcagagaggatcttccgatggggctcggggtggcgtgatctcccggcccccaggcgtccagtacccacaccccagaaggcttccaccttcacgtggacgcgcaggctgccggtgggctcccgttctctctctctttctgaggctagaggactgagccagtccttggctccccagagacatcacggcccgcagccccggagccaagtgccccgagtcccaggcgtccatgtccttcccgaggccgcgcgcacctctcctcgccccgatgggcacccactgctctgcgtggctgcggtggccgctgttgccgctgttgccgccgctgctgctgctgttgctgctactgtgccccaccggcgctggtgcccaggacgaggatggagattatgaagagctgatgctcgccctcccgtcccaggaggatggcctggctgatgaggccgcacatgtggccaccgccaccttccgccgttgctccaaggaggcctggaggctgccaggaacctacattgtggtgctgatggaggagacccagaggctacagattgaacaaactgcccaccgcctgcagacccgggctgcccgccggggctatgtcatcaaggttctacatatcttttatgacctcttccctggcttcttggtgaagatgagcagtgacctgttgggcctggccctgaagttgccccatgtggagtacattgaggaagactcctttgtcttcgcccagagcatcccatggaacctggagcgaattatcccagcatggcaccagacagaggaagaccgctcccctgatggaagcagccaggtggaggtgtatctcttagataccagcatccagggtgcccatcgggagattgagggcagggtcaccatcaccgacttcaacagcgtgccggaggaggatgggacacgcttccacagacaggcgagcaagtgtgacagccacggcacccacctggcaggtgtggtcagcggccgggatgctggtgtggccaagggcaccagcctgcacagcctgcgtgtgctcaactgtcaagggaagggcacagtcagcggcaccctcataggcctggagtttattcggaagagtcagctaatccagccctcggggccactcgtggttctgctgcccctggccggtgggtatagccgcatcctcaacgctgcctgccggcacctggcgaggactggggtggtgctggttgcagcagctgggaacttccgggacgacgcctgcctctactccccagcttctgctccagaggtcatcacagtcggggccacgaatgcccaggaccagccagttaccttggggactttggggactaattttggacgctgtgtggatctctttgcccccgggaaggacatcatcggagcgtccagtgactgcagcacatgcttcatgtcacagagtgggacctcacaggctgctgcccacgtggccggcattgtggctcggatgctgagccgggagcccacacttaccctggccgagctgcggcagaggctgatccacttctctaccaaagacgtcatcaacatggcctggttccctgaggaccagcaggtgctgacccccaacctggtggccacactgccccccagcacccatgagacaggcgggcagctgctctgtaggacggtgtggtcggcacactcggggcccactcgaacagctacagctacagcccgctgtgccccagaagaggagctgctgagctgctccagcttctccaggagcgggaggcgtcgtggtgattggattgaggccataggaggccagcaggtctgcaaggccctcaatgcatttgggggtgagggtgtctatgccgtcgcgagatgctgcctggttccccgtgccaactgcagcatccacaacacccctgcagccagagctggcctggagacccatgtccactgccaccagaaggaccatgttctcacaggctgcagcttccattgggaagtggaagaccttagtgtccggaggcagcctgcgctgaggtccagacgtcagcctggccagtgcgttggccaccaggcggccagtgtctatgcttcctgctgccatgccccagggctggaatgcaaaatcaaggagcatgggatctcaggtccttcagagcaggtcactgtggcctgcgaagcaggatggaccctgactggatgcaatgtgctccctggggcatccctcactctgggagcctacagcgtggacaacctgtgtgtggcaagagtccatgacactgccagagcagacaggaccagtggagaagccacagtagctgctgccatctgctgccggagccggccttcagcaaaggcctcctgggttcagtgacagcctcaggcagggatggtgcttgaggctgggtgcagagatatgcctgcatggctctcttgtagccaaaggtggggagattctgcgtgggagaacttggtgtctcaccctgggtacccattcctggtgtatggaagcacctccttcacggtcagggggcctgtgcttggctttctgccatcagacattaagctgtagctggctctggccagctgctccagtgtaccagaacctgaggatgctcgctgcaaggcctcagttctcaggccttagggtgtatttgtctttcaggaagatcataatggacagagatccttggaggttcaaagaccaagtaccagactggaaaattgagtctgaaagccacaaggacagtcaactcacagccagctcacattgcagacaccattttgggctccctgattaaatgcagatcagttctgcacacctccaggggtggatccagctgtaaggccatacctatatcttccagatgtcctcatctgctgcagggctttggccctgctcaggataatgtgctatgagccctcatctgactctcagtttgtactggagaaccatacaggacttaccgcaccttaccccatccactaccatgtgcactgactggcctcattttatgaaggaagagacaggaccagagaggcgatgtcacacagccagtgatgtcaggacataaattcagagtggctggccctgaataatgccaggctgggcagcgagaggacaggctatggcttgctcctggacctatactcccttagccccagtcccacagatcaggtggagactggagtgacagagggcgactgtaccaaggccacaccagctgaccagcacacctctatccttttgagctcttctgtctttttatagtaagcttcctccacctgtgttgcttttgtaacttgatatttatgcagggttttgtagtttttattatgtagtgacttttcagaataaaagcagctgatgtgactgactgcatccg

SEQ ID NO: 58 - DNA, 3731 bp, Homo sapiens
  source 1..3731: mol_type = other DNA
gtccgatggggctctggtggcgtgatctgcgcgccccaggcgtcaagcacccacaccctagaaggtttccgcagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaacaaaaaaaaaaaaaaaaaaaaa

SEQ ID NO: 59 - DNA, 19 bp, Synthetic construct
  source 1..19: mol_type = other DNA
  modified_base 8: mod_base = OTHER; note = 7-deaza-G
gtagaaggggatgaagcag

SEQ ID NO: 60 - skipped

SEQ ID NO: 61 - DNA, 19 bp, Synthetic construct
  source 1..19: mol_type = other DNA
  modified_base 8: mod_base = OTHER; note = 7-deaza-G
gtagaaggggatgaagcag

SEQ ID NO: 62 - DNA, 19 bp, Synthetic construct
  source 1..19: mol_type = other DNA
  modified_base 8: mod_base = OTHER; note = 7-deaza-G
gtagaaggggatgaagcag

SEQ ID NO: 63 - DNA, 19 bp, Synthetic construct
  source 1..19: mol_type = other DNA
  modified_base 8: mod_base = OTHER; note = 7-deaza-G
gtagaaggggatgaagcag

SEQ ID NO: 64 - DNA, 19 bp, Synthetic construct
  source 1..19: mol_type = other DNA
  modified_base 8: mod_base = OTHER; note = 7-deaza-G
gtagaaggggatgaagcag

SEQ ID NO: 65 - DNA, 19 bp, Synthetic construct
  source 1..19: mol_type = other DNA
  modified_base 8: mod_base = OTHER; note = 7-deaza-G
gtagaaggggatgaagcag

SEQ ID NO: 66 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 10..19: note = DNA
  modified_base 9: mod_base = OTHER; note = 2 prime OME phosphorothioate
  misc_feature 1..9: note = RNA
gtagaaggggatgaagcag

SEQ ID NO: 67 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 10..19: note = DNA
  modified_base 9: mod_base = OTHER; note = 2 prime OME phosphorothioate
  misc_feature 1..9: note = RNA
gtagaaggggatgaagcag

SEQ ID NO: 68 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 9..19: note = DNA
  misc_feature 1..8: note = RNA
gtagaaggggatgaagcag

SEQ ID NO: 69 - DNA, 19 bp, synthetic construct
  source 1..19: mol_type = other DNA
  modified_base 1..7: mod_base = OTHER; note = 2 prime OME phosphorothioate
  modified_base 8: mod_base = OTHER; note = 7-deaza-G
  misc_feature 9..19: note = DNA
  misc_feature 1..8: note = RNA
gtagaaggggatgaagcag

SEQ ID NO: 70 - DNA, 19 bp, Synthetic construct
  source 1..19: mol_type = other DNA
gtagaagggcatgaagcag

SEQ ID NO: 71 - DNA, 19 bp, Synthetic construct
  source 1..19: mol_type = other DNA
gtagaagggcatgaagcag

SEQ ID NO: 72 - DNA, 19 bp, Synthetic construct
  source 1..19: mol_type = other DNA
gtagaagggcatgaagcag

SEQ ID NO: 73 - DNA, 19 bp, Synthetic construct
  source 1..19: mol_type = other DNA
gtagaagggcatgaagcag

SEQ ID NO: 74 - DNA, 19 bp, Synthetic construct
  source 1..19: mol_type = other DNA
gtagaagggcatgaagcag

SEQ ID NO: 75 - DNA, 19 bp, Synthetic construct
  source 1..19: mol_type = other DNA
gtagaagggcatgaagcag

SEQ ID NO: 76 - DNA, 19 bp, synthetic construct
  source 1..19: mol_type = other DNA
  misc_feature 1..9: note = RNA
  misc_feature 10..19: note = DNA
  modified_base 1..9: mod_base = OTHER; note = 2 prime OME phosphorothioate
gtagaagggcatgaagcag

SEQ ID NO: 77 - DNA, 19 bp, synthetic construct
  source 1..19: mol_type = other DNA
  misc_feature 1..9: note = RNA
  misc_feature 10..19: note = DNA
  modified_base 1..9: mod_base = OTHER; note = 2 prime OME phosphorothioate
gtagaagggcatgaagcag

SEQ ID NO: 78 - DNA, 19 bp, synthetic construct
  source 1..19: mol_type = other DNA
  misc_feature 1..8: note = RNA
  misc_feature 9..19: note = DNA
  modified_base 1..8: mod_base = OTHER; note = 2 prime OME phosphorothioate
gtagaagggcatgaagcag

SEQ ID NO: 79 - DNA, 19 bp, synthetic construct
  source 1..19: mol_type = other DNA
  misc_feature 1..8: note = RNA
  misc_feature 9..19: note = DNA
  modified_base 1..8: mod_base = OTHER; note = 2 prime OME phosphorothioate
gtagaagggcatgaagcag

SEQ ID NO: 80 - DNA, 4649 bp, Mus musculus
  source 1..4649: mol_type = other DNA
agagcagcaacaccgggagcagagctgaactgcagcgccgcccggagcttcaagcaccatgtatgacccagagcgccgctggagcctgtcgtttgcaggctgcggcttcctgggcttctaccacgtcggggctacgctatgtctgagcgagcgcgccccgcacctcctccgcgatgcgcgcactttctttggctgctcggccggtgcactgcacgcggtcaccttcgtgtgcagtctccctctcggccgtataatggagatcctcatggacctcgtgcggaaagccaggagccgcaacatcggcaccctccacccgttcttcaacattaacaagtgcatcagagacgggctccaggagagcctcccagacaatgtccaccaggtcatttctggcaaggttcacatctcactcaccagggtgtcggatggggagaacgtgctggtgtctgagttccattccaaagacgaagtcgtggatgccctggtgtgttcctgcttcattcccctcttctctggcctaatccctccttccttccgaggcgagcggtacgtggacggaggagtgagcgacaacgtccctgtgctggatgccaaaaccaccatcacggtgtcacctttctacggtgagcatgacatctgccccaaagtcaagtccaccaacttcttccacgtgaatatcaccaacctcagcctccgcctctgcactgggaacctccaacttctgaccagagcgctcttcccgtctgatgtgaaggtgatgggagagctgtgctatcaagggtacctggacgccttccggttcctggaggagaatggcatctgtaacgggccacagcgcagcctgagtctgtccttggtggcgccagaagcctgcttggaaaatggcaaacttgtgggagacaaggtgccagtcagcctatgctttacagatgagaacatctgggagacactgtcccccgagctcagcacagctctgagtgaagcgattaaggacagggagggctacctgagcaaagtctgcaacctcctgcccgtcaggatcctgtcctacatcatgctgccctgcagtctgcccgtggagtcggctatcgctgcagtccacaggctggtgacatggctccctgatatccaggatgatatccagtggctacaatgggcgacatcccaggtttgtgcccgaatgacgatgtgcctgctcccctctaccaggtaaatacttgggcccagggtgtgtgggccagataggcatccctcccggttgttcccagagctcttagggtcagagcttgggtggtgacagccttaacaagccaggctcagccgcctgtccccagcatgccattaaagaaaccggtagcagagaaagcaggtttattcgaatataaaaaggttcaagcccccacccggttaatctttaagataccaacaggaggcttaagtttaaacagagttacacataaacagtctgaatcagggcgtggtcctgcccaccattgtctgggcttcaaggttccttctttctctccctagcatgagattcctgggacaatcccaattccttggcctccattgtatcaaagggctgaaaaccaaagggaaggcacagctgtctcttcagcatgcctcttctgccagaaccactgcaaggtttggtgctcaggctgtgcaaacattctagcaatgtttgactcagtgtcaagcaggtgacaaggaacatggtgctgtgtggggggaacccatggcccaggtgagggcttattggtgggtgaagctgtgggtgttcaggtggtggagaaggccttaagggatgggactgacacctcagcactgaaggcaggaggaagctgtggctctgggttgcacccctgcctggctccaccctctctggcatctgtagaagttacagctggttcttcctctcagccccatgctcccagaaataagactcagacccaaattatagttacaaataccttggccatatagctaggctcttctcagactagctcataacttaactcattaattttaacctccatcctgccacatggctggtggcctgtgctcaggtaccatgagtccagctcttcacatctttccggatgaatcttccataattctttctgcctcctggatgttccaccttctattccaccttttcctataggccatggttttgtttttgttttttttttccaaatttaatttaattaattaatttatttatttttggtttttcgagacagggtttctctgtatcgccctggctgtcctggaactcactatgtaagccaggctggcctcaaactcagaaatccgcctgcctctgcctcctgagtgctgggattaaaggcgtgcgcaaccatgcccggtgtggtttttttttttttttaattgacaggtggatgcatctatataatccataacatattctctctacaggtatctattaggttttgggtgaggtgtggagttctagggaactctgagagaaattcctggggagtaagtggtttatcaagttgattggaggagtttttaatgctatggacagacagacagaaggacaacagcatagtcggggctaccagggagttcaggccccggcatcggagatagaagcaggatggggtctttgaagagattctgagcccacacagcagaggagggactctctctttagagcttttgaggatgagggaggttgactgcaagagcctacagccaggctcgaggcaggcagggggtggggagcaggatgtaaaccccttcgatgctgacagactcacttctggggtaaaatattatgagatgcctgtcagtgtctgtgaagagacctgagcagagtctggattctgacatcaatcatgttcttacaatactgaagacctgagagcctgcaatcttggtttgtaaattgctggtctccgtgcttccagtgaacttggacattcttctcatggttggtccaggagaggccaaagctgagggcaccctgccttccacccccagtccagcttgaccttttatctggagcaacagtgtctagatgatgggtgggtgaggggtgctatactgtctgtccctctgggaagggttctgttacttttggaggcagctaggaagtttctctgtgcagctgccccctggtgctgtgtggtgacctcattgcctgtgaccccaggatcacaggatctgggctaaagtggtagtccatagaaaccaaagacaatgatttggtgtttagaaagctactcttggtctgggtgaagtctggtgcttaagggctatcacaaagagcgtgtcaaaccatctctcagcctgtgagtcagtggggagcccaagggcatcagtgtttggaaactggaatccaaaccgggcaatctcggaaggaaactgtttaggaattgtgatgggacgggccgtggctgtctctgaaaagggcctgccagataacttattacttttaaggacacctttggctcttactaatttataaagcattttatataaacacaccagggagtgcatggtgaactacacgtatgatcagttaagtggggctagaattaggtagggagagcatcggacctctgcctcctcaacctcaacttgcttgctttctccactggctccaaatctttgtatagtcatcagccatgaccacctctctccctccccatctactaccagcagcgttaatgggaataagtacccacttctctcaggtgtactatacagctgtgggtgtggtgtgtgtttcctgtaattcacactttagaaaggaaacaagcaaacaaaagaaaccaggtgctgcccatactcctaagtgtagacagtgaaggtgtgtgtctcccatgcctgagtctcctggaggcctagtgagctccaggttcatgcaagcacatcaggaggaatcatataatctcagcacggttgatccagatgggataagaaaggactctgggagagagaatgtggttctagagacaaagtgtctaggctacacagaagataagactgtcccaaggaaagaaaagaaaccaggaactagggtgcagctcagttgtcagaggacttctctaggcttgaagcccagagtccaatctcagcaccttataaactgtggagtgacaggcagtgacatcggcctgtaatcccaacactcaagcagtagaggcaagaggatcataagttcaaggtcttccttggctatttagggagttggaggttagctctggctacatgagaccctgtctcaaaaaaaaaaaaaaaaaaaagtagaaacttctgccttgctttgagctgcccctttctggacgtttctcatcagtagagaatattcctgccaccctatcagacaaaactcccactggtttggagtctctccattctcaggaacacctcaggagtcagacagtgagcagcagggagcaatgtcttgacttgtaagccccttagcaaggctggttcatttgtttattaaaagcaggtgtgggtgaatttatgcaaatgagtatgcaaactagtggaacagcagaaggattgaatggatacaccaaaaataaccacaactgtttaagggaaaagggtccataataaatgtggggaacaaaaaacaaataaatgtgattttttttagaaaaatg

SEQ ID NO: 81 - DNA, 2805 bp, Homo sapiens
  source 1..2805: mol_type = other DNA
atggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagac

SEQ ID NO: 82 - DNA, 2291 bp, Macaca mulatta
  source 1..2291: mol_type = other DNA
cgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccgctgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagcagtgagtgaagcaatgaaagacaaaggtggatacatgagcaagatttgcaacttgctacccattaggataatgtcttatgtgatgctgccctgtaccctgcctgtggagtctgccattgcgattgtccagagactggtgacatggcttccggatatgcccgacgatgtgcagtggctgcagtgggtgacctcacaggtcttcactcgagcgctgatgtgtctgcttcccgcctccaggtcccaaatgccagtgagcggcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatttgtgagtcatttgaggaggcgagtctaggagattctttcagaggtgctaaagcttcccatctttgtgcagctacctccgcattgccgtgtagtgacccctgcctgtgacgtggaggatcccagcctctgagctgagttggttttatgaaaagctaggaagcaatgtttggtctgtgcagcagtccagcacttaagtctaatacgtcagcatgcgttagttcagctggttgggaaatgacaccgggaagcctagcgcagagggtcccttactgactatttcatggtcctattaatggtcagactgttccagtgtgaggttcttagaatgactagtgtttggatgggtgggggccttgtggtggggggtgggctggcctatgtgtgatcttgtggggtggaaggaagagagtagcacaatcccacctccccatgccgtgggaaggggtgcacttggttcccaagaaggacactgcctgtcaggtggcctgcaaatataataaccttgacaactaaaaacctctccatgggggtggggggtaccaagataataaccgatttacattttagagcacctttttcacctaactaaaataatgtttaaagagttttatataaaaatgtaaggaattgttatctgttgaattttgtattatatgaatcagtgagatgttaatagaataagcctt

SEQ ID NO: 83 - DNA, 2294 bp, Macaca fascicularis
  source 1..2294: mol_type = other DNA
cgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccggtgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagcagtgagtgaagcaatgaaagacaaaggtggatacatgagcaagatttgcaacttgctacccattaggataatatcttatgtgatgctgccctgtaccctgcctgtggagtctgccattgcgattgtccagagactggtgacatggcttccagatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatttgtgagtcatttgaggaggcgagtctaggagattctttcagaggtgctaaagcttcccatctttgtgcagctacctccgcattgccgtgtagtgacccctgcctgtgacgtggaggatcccagcctctgagctgagttggttttatgaaaagctaggaagcaatgtttggtctgtgcagcagtccagcacttaagtctaatacgtcagcatgcgttagttcagctggttgggaaatgacaccgggaagcctagcgcagagggtcccttactgactatttcatggtcctattaatggtcagactgttccagtgtgaggttcttagaatgactagtgtttggatgggtgggggccttgtggtggggggtgggctggcctatgtgtgatcttgtggggtggaaggaagagagtagcacaatcccacctccccatgccgtgggaaggggtgcacttggttcccaagaaggacactgcctgtcaggtggcctgcaaatataataaccttgacaactaaaaacctctccatgggggtgggaggtaccaagataataaccgatttacattttagagcacctttttcacctaactaaaataatgtttaaagagttttatataaaaatgtaaggaagagttgttatctgttgaattttgtattatatgaatcagtgagatgttaatagaataagcctt

SEQ ID NO: 84 - DNA, 25 bp, Synthetic construct
  source 1..25: mol_type = other DNA
  misc_feature 1..25: note = DNA phosphodiester
tcgtaacgttacgtattcgaatcgt

SEQ ID NO: 85 - DNA, 25 bp, Synthetic construct
  source 1..25: mol_type = other DNA
  misc_feature 1..25: note = DNA phosphodiester
acgattcgaatacgtaacgttacga

SEQ ID NO: 86 - DNA, 25 bp, Synthetic construct
  source 1..25: mol_type = other DNA
tcgtatcgtaacgtattcgaatcgt

SEQ ID NO: 87 - DNA, 25 bp, Synthetic construct
  source 1..25: mol_type = other DNA
  misc_feature 1..6: note = DNA phosphodiester
tcgtatcgtaacgtattcgaatcgt

SEQ ID NO: 88 - DNA, 27 bp, Synthetic construct
  source 1..27: mol_type = other DNA
tcgacttgaacgattcgaatacgtaac

SEQ ID NO: 89 - RNA, 27 nt, synthetic construct
  source 1..27: mol_type = other RNA
  modified_base 6: mod_base = OTHER; note = uracil
tcgacttgaacgattcgaatacgtaac

SEQ ID NO: 90 - DNA, 27 bp, Synthetic construct
  source 1..27: mol_type = other DNA
  modified_base 8: mod_base = i; note = deoxy inosine
tcgacttnaacgattcgaatacgtaac

SEQ ID NO: 91 - DNA, 27 bp, Synthetic construct
  source 1..27: mol_type = other DNA
  misc_feature 1..6: note = DNA phosphodiester
tcgacttgaacgattcgaatacgtaac

SEQ ID NO: 92 - DNA, 27 bp, Synthetic construct
  source 1..27: mol_type = other DNA
tcgacttgaacgattcgaatacgtaac

SEQ ID NO: 93 - DNA, 27 bp, Synthetic construct
  source 1..27: mol_type = other DNA
tcgacttgaacgattcgaatacgtaac

SEQ ID NO: 94 - DNA, 27 bp, Synthetic construct
  source 1..27: mol_type = other DNA
tcgacttgaacgattcgaatacgtaac

SEQ ID NO: 95 - DNA, 27 bp, Synthetic construct
  source 1..27: mol_type = other DNA
tcgacttgaacgattcgaatacgtaac

SEQ ID NO: 96 - DNA, 27 bp, Synthetic construct
  source 1..27: mol_type = other DNA
  misc_feature 1..6: note = DNA phosphodiester
tcgacttgaacgattcgaatacgtaac

SEQ ID NO: 97 - DNA, 27 bp, Synthetic construct
  source 1..27: mol_type = other DNA
tcgacttgaacgattcgaatacgtaac

SEQ ID NO: 98 - DNA, 27 bp, Synthetic construct
  source 1..27: mol_type = other DNA
tcgacttgaacgattcgaatacgtaac

SEQ ID NO: 99 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 100 - DNA, 27 bp, Synthetic construct
  source 1..27: mol_type = other DNA
  misc_feature 9..27: note = DNA phosphodiester
tcgacttgaacgattcgaatacgtaac

SEQ ID NO: 101 - DNA, 27 bp, Synthetic construct
  source 1..27: mol_type = other DNA
tcgacttgaacgattcgaatacgtaac

SEQ ID NO: 102 - DNA, 27 bp, Synthetic construct
  source 1..27: mol_type = other DNA
tcgacttgaacgattcgaatacgtaac

SEQ ID NO: 103 - DNA, 27 bp, Synthetic construct
  source 1..27: mol_type = other DNA
tcgacttgaacgattcgaatacgtaac

SEQ ID NO: 104 - DNA, 27 bp, Synthetic construct
  source 1..27: mol_type = other DNA
tcgacttgaacgattcgaatacgtaac

SEQ ID NO: 105 - DNA, 27 bp, Synthetic construct
  source 1..27: mol_type = other DNA
tcgacttgaacgattcgaatacgtaac

SEQ ID NO: 106 - DNA, 27 bp, Synthetic construct
  source 1..27: mol_type = other DNA
gttacgtattcgaatcgttcaagtcga

SEQ ID NO: 107 - DNA, 27 bp, Synthetic construct
  source 1..27: mol_type = other DNA
gttacgtattcgaatcgttcaagtcga

SEQ ID NO: 108 - DNA, 27 bp, Synthetic construct
  source 1..27: mol_type = other DNA
gttacgtattcgaatcgttcaagtcga

SEQ ID NO: 109 - DNA, 25 bp, Synthetic construct
  source 1..25: mol_type = other DNA
  misc_feature 1..25: note = DNA phosphodiester
tcgtatcgtaacgtattcgaatcgt

SEQ ID NO: 110 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 111 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 112 - DNA, 27 bp, Synthetic construct
  source 1..27: mol_type = other DNA
  misc_feature 1..27: note = DNA phosphodiester
gttacgtattcgaatcgttcaagtcga

SEQ ID NO: 113 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 114 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 115 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 116 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 117 - DNA, 27 bp, Synthetic construct
  source 1..27: mol_type = other DNA
  misc_feature 1..27: note = DNA phosphodiester
gttacgtattcgaatcgttcaagtcga

SEQ ID NO: 118 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 119 - DNA, 27 bp, Synthetic construct
  source 1..27: mol_type = other DNA
tcgacttgaacgattcgaatacgtaac

SEQ ID NO: 120 - DNA, 27 bp, Synthetic construct
  source 1..27: mol_type = other DNA
tcgacttgaacgattcgaatacgtaac

SEQ ID NO: 121 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 122 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 123 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 124 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 125 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 126 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 127 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 128 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 129 - DNA, 27 bp, Synthetic construct
  source 1..27: mol_type = other DNA
  modified_base 2: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 22: mod_base = OTHER; note = 5 prime -methylated C
tcgacttgaacgattcgaatacgtaac

SEQ ID NO: 130 - DNA, 19 bp, synthetic construct
  source 1..19: mol_type = other DNA
  modified_base 1..7: mod_base = OTHER; note = 2 prime OME phosphorothioate
  modified_base 8: mod_base = OTHER; note = 7-deaza-G
  misc_feature 10..19: note = DNA
  modified_base 9: mod_base = OTHER; note = 2 prime OME phosphorothioate
  misc_feature 1..9: note = RNA
gtagaaggggatgaagcag

SEQ ID NO: 131 - DNA, 19 bp, synthetic construct
  source 1..19: mol_type = other DNA
  modified_base 1..7: mod_base = OTHER; note = 2 prime OME phosphorothioate
  modified_base 8: mod_base = OTHER; note = 7-deaza-G
  misc_feature 10..19: note = DNA
  modified_base 9: mod_base = OTHER; note = 2 prime OME phosphorothioate
  misc_feature 1..9: note = RNA
gtagaaggggatgaagcag

SEQ ID NO: 132 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 9..19: note = DNA
  misc_feature 1..8: note = RNA
gtagaaggggatgaagcag

SEQ ID NO: 133 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 9..19: note = DNA
  misc_feature 1..8: note = RNA
gtagaaggggatgaagcag

SEQ ID NO: 134 - DNA, 19 bp, synthetic construct
  source 1..19: mol_type = other DNA
  modified_base 1..9: mod_base = OTHER; note = 2 prime OME phosphorothioate
  misc_feature 10..19: note = DNA
  misc_feature 1..9: note = RNA
gtagaagggcatgaagcag

SEQ ID NO: 135 - DNA, 19 bp, synthetic construct
  source 1..19: mol_type = other DNA
  modified_base 1..9: mod_base = OTHER; note = 2 prime OME phosphorothioate
  misc_feature 10..19: note = DNA
  misc_feature 1..9: note = RNA
gtagaagggcatgaagcag

SEQ ID NO: 136 - DNA, 19 bp, synthetic construct
  source 1..19: mol_type = other DNA
  misc_feature 1..8: note = RNA
  misc_feature 9..19: note = DNA
  modified_base 1..8: mod_base = OTHER; note = 2 prime OME phosphorothioate
gtagaagggcatgaagcag

SEQ ID NO: 137 - DNA, 19 bp, synthetic construct
  source 1..19: mol_type = other DNA
  misc_feature 1..8: note = RNA
  misc_feature 9..19: note = DNA
  modified_base 1..8: mod_base = OTHER; note = 2 prime OME phosphorothioate
gtagaagggcatgaagcag

SEQ ID NO: 138 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 10..19: note = DNA
  modified_base 9: mod_base = OTHER; note = 2 prime OME phosphorothioate
  misc_feature 1..9: note = RNA
gtagaaggggatgaagcag

SEQ ID NO: 139 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 10..19: note = DNA
  modified_base 9: mod_base = OTHER; note = 2 prime OME phosphorothioate
  misc_feature 1..9: note = RNA
gtagaaggggatgaagcag

SEQ ID NO: 140 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 9..19: note = DNA
  misc_feature 1..8: note = RNA
gtagaaggggatgaagcag

SEQ ID NO: 141 - DNA, 19 bp, synthetic construct
  source 1..19: mol_type = other DNA
  modified_base 8: mod_base = OTHER; note = 7-deaza-G
  modified_base 1..7: mod_base = OTHER; note = 2 prime OME phosphorothioate
  misc_feature 9..19: note = DNA
  misc_feature 1..8: note = RNA
gtagaaggggatgaagcag

SEQ ID NO: 142 - DNA, 19 bp, synthetic construct
  source 1..19: mol_type = other DNA
  modified_base 1..9: mod_base = OTHER; note = 2 prime OME phosphorothioate
  misc_feature 10..19: note = DNA
  misc_feature 1..9: note = RNA
gtagaagggcatgaagcag

SEQ ID NO: 143 - DNA, 19 bp, synthetic construct
  source 1..19: mol_type = other DNA
  modified_base 1..9: mod_base = OTHER; note = 2 prime OME phosphorothioate
  misc_feature 10..19: note = DNA
  misc_feature 1..9: note = RNA
gtagaagggcatgaagcag

SEQ ID NO: 144 - DNA, 19 bp, synthetic construct
  source 1..19: mol_type = other DNA
  misc_feature 1..8: note = RNA
  misc_feature 9..19: note = DNA
  modified_base 1..8: mod_base = OTHER; note = 2 prime OME phosphorothioate
gtagaagggcatgaagcag

SEQ ID NO: 145 - DNA, 19 bp, synthetic construct
  source 1..19: mol_type = other DNA
  modified_base 1..8: mod_base = OTHER; note = 2 prime OME phosphorothioate
  misc_feature 9..19: note = DNA
  misc_feature 1..8: note = RNA
gtagaagggcatgaagcag

SEQ ID NO: 146-173 - skipped (28 entries)

SEQ ID NO: 174 - DNA, 25 bp, Synthetic construct
  source 1..25: mol_type = other DNA
acgattcgaatacgttacgatacga

SEQ ID NO: 175 - DNA, 25 bp, Synthetic construct
  source 1..25: mol_type = other DNA
  misc_feature 1..6: note = DNA phosphodiester
acgattcgaatacgttacgatacga

SEQ ID NO: 176 - DNA, 27 bp, Synthetic construct
  source 1..27: mol_type = other DNA
gttacgtattcgaatcgttcaagtcga

SEQ ID NO: 177 - DNA, 27 bp, Synthetic construct
  source 1..27: mol_type = other DNA
gttacgtattcgaatcgttcaagtcga

SEQ ID NO: 178 - DNA, 27 bp, Synthetic construct
  source 1..27: mol_type = other DNA
gttacgtattcgaatcgttcaagtcga

SEQ ID NO: 179 - DNA, 27 bp, Synthetic construct
  source 1..27: mol_type = other DNA
  misc_feature 1..6: note = DNA phosphodiester
gttacgtattcgaatcgttcaagtcga

SEQ ID NO: 180 - DNA, 21 bp, Synthetic construct
  source 1..21: mol_type = other DNA
tattcgaatcgttcaagtcga

SEQ ID NO: 181 - DNA, 18 bp, Synthetic construct
  source 1..18: mol_type = other DNA
tcgaatcgttcaagtcga

SEQ ID NO: 182 - DNA, 13 bp, Synthetic construct
  source 1..13: mol_type = other DNA
tcgttcaagtcga

SEQ ID NO: 183-184 - skipped (2 entries)

SEQ ID NO: 185 - DNA, 27 bp, Synthetic construct
  source 1..27: mol_type = other DNA
gttacgtattcgaatcgttcaagtcga

SEQ ID NO: 186 - DNA, 27 bp, Synthetic construct
  source 1..27: mol_type = other DNA
  misc_feature 1..27: note = DNA phosphodiester
gttacgtattcgaatcgttcaagtcga

SEQ ID NO: 187 - DNA, 27 bp, Synthetic construct
  source 1..27: mol_type = other DNA
  misc_feature 8..27: note = DNA phosphodiester
gttacgtattcgaatcgttcaagtcga

SEQ ID NO: 188 - DNA, 21 bp, Synthetic construct
  source 1..21: mol_type = other DNA
tattcgaatcgttcaagtcga

SEQ ID NO: 189 - DNA, 18 bp, Synthetic construct
  source 1..18: mol_type = other DNA
tcgaatcgttcaagtcga

SEQ ID NO: 190 - DNA, 13 bp, Synthetic construct
  source 1..13: mol_type = other DNA
tcgttcaagtcga

SEQ ID NO: 191 - DNA, 13 bp, Synthetic construct
  source 1..13: mol_type = other DNA
  misc_feature 1..13: note = DNA phosphodiester
tcgttcaagtcga

SEQ ID NO: 192 - skipped

SEQ ID NO: 193 - DNA, 27 bp, Synthetic construct
  source 1..27: mol_type = other DNA
tcgacttgaacgattcgaatacgtaac

SEQ ID NO: 194 - skipped

SEQ ID NO: 195 - DNA, 25 bp, Synthetic construct
  source 1..25: mol_type = other DNA
  misc_feature 1..25: note = DNA phosphodiester
acgattcgaatacgttacgatacga

SEQ ID NO: 196 - DNA, 27 bp, Synthetic construct
  source 1..27: mol_type = other DNA
  misc_feature 1..27: note = DNA phosphodiester
gttacgtattcgaatcgttcaagtcga

SEQ ID NO: 197 - DNA, 21 bp, Synthetic construct
  source 1..21: mol_type = other DNA
  misc_feature 1..21: note = DNA phosphodiester
tattcgaatcgttcaagtcga

SEQ ID NO: 198 - DNA, 27 bp, Synthetic construct
  source 1..27: mol_type = other DNA
  misc_feature 1..27: note = DNA phosphodiester
tcgacttgaacgattcgaatacgtaac

SEQ ID NO: 199 - DNA, 27 bp, Synthetic construct
  source 1..27: mol_type = other DNA
  misc_feature 1..27: note = DNA phosphodiester
gttacgtattcgaatcgttcaagtcga

SEQ ID NO: 200 - DNA, 25 bp, Synthetic construct
  source 1..25: mol_type = other DNA
  misc_feature 1..25: note = DNA phosphodiester
tacgtattcgaatcgttcaagtcga

SEQ ID NO: 201 - DNA, 24 bp, Synthetic construct
  source 1..24: mol_type = other DNA
  misc_feature 1..24: note = DNA phosphodiester
acgtattcgaatcgttcaagtcga

SEQ ID NO: 202 - DNA, 27 bp, Synthetic construct
  source 1..27: mol_type = other DNA
  misc_feature 1..27: note = DNA phosphodiester
gttagctattgcaatgcttcaagtgca

SEQ ID NO: 203 - RNA, 27 nt, synthetic construct
  source 1..27: mol_type = other RNA
  misc_feature 1..27: note = RNA phosphodiester
gttacgtattcgaatcgttcaagtcga

SEQ ID NO: 204 - RNA, 18 nt, synthetic construct
  source 1..18: mol_type = other RNA
  misc_feature 1..18: note = RNA phosphodiester
tcgaatcgttcaagtcga

SEQ ID NO: 205 - DNA, 27 bp, Synthetic construct
  source 1..27: mol_type = other DNA
  modified_base 5: mod_base = OTHER; note = 5 prime -methylated C
  modified_base 11: mod_base = OTHER; note = 5 prime -methylated C
  modified_base 16: mod_base = OTHER; note = 5 prime -methylated C
  modified_base 25: mod_base = OTHER; note = 5 prime -methylated C
gttacgtattcgaatcgttcaagtcga

SEQ ID NO: 206 - DNA, 21 bp, Synthetic construct
  source 1..21: mol_type = other DNA
  modified_base 5: mod_base = OTHER; note = 5 prime -methylated C
  modified_base 10: mod_base = OTHER; note = 5 prime -methylated C
  modified_base 19: mod_base = OTHER; note = 5 prime -methylated C
tattcgaatcgttcaagtcga

SEQ ID NO: 207 - DNA, 18 bp, Synthetic construct
  source 1..18: mol_type = other DNA
  modified_base 2: mod_base = OTHER; note = 5 prime -methylated C
  modified_base 7: mod_base = OTHER; note = 5 prime -methylated C
  modified_base 16: mod_base = OTHER; note = 5 prime -methylated C
tcgaatcgttcaagtcga

SEQ ID NO: 208 - DNA, 13 bp, Synthetic construct
  source 1..13: mol_type = other DNA
  modified_base 2: mod_base = OTHER; note = 5 prime -methylated C
  modified_base 11: mod_base = OTHER; note = 5 prime -methylated C
tcgttcaagtcga

SEQ ID NO: 209 - skipped

SEQ ID NO: 210 - DNA, 11 bp, Synthetic construct
  source 1..11: mol_type = other DNA
gttcaagtcga

SEQ ID NO: 211-216 - skipped (6 entries)

SEQ ID NO: 217 - RNA, 27 nt, synthetic construct
  source 1..27: mol_type = other RNA
  misc_feature 1..27: note = RNA phosphodiester
tcgacttgaacgattcgaatacgtaac

SEQ ID NO: 218 - DNA, 18 bp, synthetic construct
  source 1..18: mol_type = other DNA
gcgtgcctcctcactggc

SEQ ID NO: 219 - DNA, 17 bp, synthetic construct
  source 1..17: mol_type = other DNA
gcgtgcctcctcacggc

(19) United States
(12) Patent Application Publication
(10) Pub. No.: US 2025/0270550 A1
(43) Pub. Date: Aug. 28, 2025

(54) Cyclic Structured Oligonucleotides as Therapeutic Agents

(71) Applicant: ARNAY Sciences, LLC, Shrewsbury, MA (US)

(72) Inventor: Sudhir AGRAWAL, Shrewsbury, MA (US)

(21) Appl. No.: 18/612,655

(22) Filed: Mar. 21, 2024

Related U.S. Application Data
(63) Continuation of application No. PCT/US22/44406, filed on Sep. 22, 2022.
(60) Provisional application No. 63/247,556, filed on Sep. 23, 2021.

(51) Int. Cl. C12N 15/113

(52) U.S. Cl. CPC C12N 15/113; C12N 2310/11; C12N 2310/321; C12N 2310/33; C12N 2310/351

(57) ABSTRACT

The present invention provides oligonucleotides referred to as cyclic structured oligonucleotides (“CSOs”) comprising a functional domain, a cyclizing domain, and a linker segment as described herein, compositions comprising same, and methods of using same. This design of cyclic oligonucleotides maintains a cyclic form until it is in the presence of and hybridizes with a targeted RNA.

### CROSS-REFERENCE TO RELATED APPLICATIONS

This application is a continuation of International Application No. PCT/US22/44406, which designated the United States and was filed on Sep. 22, 2022, published in English, which claims the benefit under 35 U.S.C. § 119(e) of the filing date of U.S. Patent Provisional Application Ser. No. 63/247,556, filed Sep. 23, 2021. The contents of the above-referenced applications are hereby incorporated herein in their entirety by reference.

### SEQUENCE LISTING

The sequence listing submitted via EFS, in compliance with 37 CFR § 1.52(e)(5), is incorporated herein by reference. The sequence listing XML file submitted via EFS contains the file 42503002 US1 Revised SEQ LIST.xml, created on Jun. 11, 2025, which is 413,007 bytes in size.

### BACKGROUND

Processing and Translation of targeted RNA can be modulated by antisense oligonucleotides by multiple mechanisms. These include cleaving the targeted RNA by RNase-H, modulating aberrant splicing, processing of targeted RNA and increased translation, inhibiting the translation by steric hindrance etc. Targeted RNA could be mRNA or noncoding RNA. In other mechanisms, the antisense strand of a duplex siRNA could be incorporated in AGO and inhibit translation by the siRNA mechanism. In other mechanisms, like ADAR or CRISPR based models, antisense could edit RNA or DNA and thereby modulate translation and processing.

Over the years it has been learned that antisense hybridization and affinity influence selectivity for the targeted RNA. In addition, to use antisense nucleic acids as drugs, nuclease stability is important which has been provided by the modification of internucleotide linkages, for example, phosphorothioate.

It was postulated that nuclease stability is key for potency and since the degradation of antisense was shown to be from the 3′-end, the focus was to modify the 3′-end to slow down degradation. These designs include capping on the 3′-end, and a hairpin loop on the 3′-end, creating oligos having secondary structures comprising 3′-3′ linkages or attaching two (2) antisense oligonucleotides at their 3′ ends. These type of antisense showed increased nuclease stability but antisense potency was not improved. Unfortunately, these modifications also increased inflammatory responses thereby limiting the therapeutic index.

Both DNA and RNA and 2′-substituted RNA containing phosphorothioate have been studied as antisense agents and provide different characteristics. DNA phosphorothioate antisense, when hybridized to RNA, activates RNase H, whereas RNA or 2′-substituted RNA antisense binds to RNA with higher affinity and does not activate RNase H. To further improve antisense characteristics, a mixture of these two modifications has been employed in antisense, generally referred to as hybrid or gapmer antisense. In most studied gapmer antisense, modified RNA segment is placed on both the 3′- and 5′-end whereas DNA is placed in the middle. Gapmer antisense is the most widely studied antisense and drugs employing this chemistry are approved and are in clinical development.

One of the side-effects of both DNA and RNA phosphorothioate is due to the interaction with proteins and more specifically with the family of Pattern Recognition Receptors (PRRs). These interactions result in induction of an immune cascade thereby causing an off-target mechanism of action and related safety signals. Detailed structure-activity relationship studies have shown that the accessibility of the 5′-end of DNA and RNA phosphorothioate antisense is required for immune activation. DNA and RNA phosphorothioate containing two 5′-ends have shown increased immuno-stimulatory activity. In contrast, it has been previously shown that DNA and RNA phosphorothioate which contain two 3′-ends (and lack 5′-) show minimal inflammatory responses.

In continuing efforts to improve the properties of DNA and modified RNA phosphorothioate antisense as therapeutic agents, structural changes in oligonucleotides were considered. For example, in earlier studies, self-stabilized oligonucleotides were reported wherein oligodeoxynucleotide phosphorothioates (“PS-oligonucleotide”) containing a hairpin loop region at the 3′-end provided increased in vivo nuclease stability and limited biological activity. To date focus has been to improve stability of antisense by modifying 3′-end by various modifications including in gapmer antisense.

Despite the advances that have been made, there is still a desire to develop antisense oligonucleotides having improved properties for use as therapeutic agents and in diagnostic applications.

### SUMMARY OF THE INVENTION

The present invention provides a structural class of oligonucleotides referred to herein as “cyclic structured oligonucleotides” (CSOs) or, equivalently, “cyclic oligos”. In CSOs, two oligonucleotides are linked to each other (directly or through a linker segment). One oligonucleotide, referred to as the “functional domain,” provides a function to the CSO (e.g., the functional segment can be an antisense oligonucleotide or an immunostimulatory oligonucleotide), and the second, referred to as the “cyclizing domain” comprises a nucleotide sequence that is complementary to a terminal end of the functional domain (e.g., FIG. 1A-FIG. 1C).

CSOs adopt an intramolecular cyclic structure as a result of complementarity between functional and cyclizing domains, which form an intramolecular duplex. This intramolecular duplex formation changes both the shape of the functional domain and accessibility to the ends of oligonucleotide. This structure combines key attributes to create optimal antisense and oligonucleotide and nucleic acid based therapeutics.

In gene and RNA expression modulation, this structure masks the 5′-end thereby reducing the interaction with PRRs and permitting endosomal escape. Once in the cytoplasm or nucleus, the cyclic structure will open in the presence of target RNA as the affinity of the functional domain and a target RNA sequence is higher than the affinity between the functional domain and cyclizing domain. When the CSO is in the intramolecular cyclic form, it may exhibit fewer of the polyanionic-related side effects (e.g., complement activation and prolongation of partial thromboplastin time) known to occur with PS-oligonucleotides, because there are fewer exposed phosphorothioate linkages. Also, CSOs would have reduced protein binding.

CSOs according to the invention can be made using standard techniques for synthesis of the constituent oligonucleotides and are useful for all purposes for which the functional oligonucleotide and nucleic acid is useful.

The foregoing merely summarizes certain aspects of the invention and is not intended, nor should it be construed, as limiting the invention in any manner. All patents, patent applications, and other publications recited in this specification are hereby incorporated by reference in their entirety.

### BRIEF DESCRIPTION OF THE DRAWINGS

FIG. 1A through FIG. 1C depicts various embodiment of the cyclic structured oligonucleotide according to the invention. The dashed line represents the cyclizing domain. The solid line represents the functional domain. L represents the linking of the functional domain and cyclizing domain directly or via a linker. As shown in FIG. 1A, the cyclic structured oligonucleotide maintains a cyclic form until it is in the presence of and hybridizes with a targeted RNA. In this embodiment, the functional domain is antisense oligonucleotide which is complementary to the targeted RNA. The cyclic structured oligonucleotides can act by various mechanisms of action depending on the nature of the oligonucleotide of the functional domain as will be further described herein. FIG. 1B depicts one embodiment of a CSO according to the invention wherein the oligonucleotide of the functional domain is a splitmer as described herein. FIG. 1C depicts one embodiment of a CSO according to the invention wherein the oligonucleotide of the functional domain is a splicing oligonucleotide as described herein.

FIGS. 2A and 2B depict various embodiments of the cyclic structured oligonucleotide according to the invention, wherein the oligonucleotide of the functional domain is a siRNA. FIG. 2A depicts one embodiment wherein the cyclizing domain is attached at the 5′-end of one strand of the siRNA. The cyclizing domain can be attached to either the antisense strand or the sense strand of the siRNA. The short dashed line represents the cyclizing domain. The solid line represents the sense strand of the siRNA. The dashed line represents the antisense strand of the siRNA. L represents the linker. FIG. 2B depicts a further embodiment of the cyclic structured oligonucleotide according to the invention, wherein the oligonucleotide of the functional domain is the antisense strand of a siRNA (dashed line), and wherein the cyclizing domain (solid line) is attached at the 5′-end. In this embodiment, the sense strand is attached to the antisense strand and also acts as the cyclizing domain. Alternatively, if FIG. 2B, the functional domain is the sense strand of a siRNA (dashed line) and the cyclizing domain (solid line) is attached at the 5′-end. In this embodiment, the antisense strand is attached to the sense strand and also acts as the cyclizing domain.

FIG. 3 depicts the delivery of a CSO according to the invention wherein the oligonucleotide of the functional domain is a gene modulation oligonucleotide, and its release from the endosome into the cytoplasm.

FIG. 4A and FIG. 4B depict exemplary embodiments of the cyclic structured oligonucleotide according to the invention, wherein the oligonucleotide of the functional domain comprises an immunostimulatory oligonucleotide and wherein a cyclizing domain of various lengths is attached at the 3′end. FIG. 4B depicts exemplary cyclic structures for compound numbers 53 and 54.

FIG. 5 depicts the knockdown of PCSK9 in Hepa1-6 cells.

FIG. 6A through FIG. 6C depict the knockdown of PNPLA3 in HepG2 cells.

FIG. 7 depicts the immunostimulatory activity of CSOs according to the invention wherein the oligonucleotide of the functional domain is a TLR9 agonists compared to non-cyclic TLR9 agonists.

FIG. 8 depicts the effect of different linkers (i.e., direct bond versus triethylene glycol) on the immunostimulatory activity of CSOs according to the invention wherein the oligonucleotide of the functional domain is a TLR9 agonists.

FIG. 9 depicts the effect of cyclizing domains of different lengths on the immunostimulatory activity of CSOs according to the invention wherein the oligonucleotide of the functional domain is a TLR9 agonists.

FIG. 10 depicts the effect of cyclizing domains of different lengths and different linkers on the immunostimulatory activity of CSOs according to the invention wherein the oligonucleotide of the functional domain is a TLR9 agonists.

FIG. 11 depicts the effect of attaching the cyclizing domains to the 5′ or 3′ of the functional domain on the immunostimulatory activity of CSOs according to the invention wherein the oligonucleotide of the functional domain is a TLR9 agonists.

FIG. 12 depicts the effect of phosphodiester internucleotide linkages on the immunostimulatory activity of CSOs according to the invention wherein the oligonucleotide of the functional domain is a TLR9 agonists.

FIG. 13 depicts the activity of CSOs according to the invention wherein the oligonucleotide of the functional domain is a TLR9 antagonist. As shown in FIG. 13, these compounds did not generate an immune response.

FIG. 14 depicts the ability of CSOs according to the invention wherein the oligonucleotide of the functional domain is a TLR9 antagonist to block the immunostimulatory activity of a TLR9 agonist.

### DETAILED DESCRIPTION

It is to be understood that both the foregoing general description and the following detailed description are exemplary and explanatory only and are not restrictive.

Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers with that range, for example, 1, 2, 2.7, 3, 4, 5, 5.3, and 6. This applies regardless of the breadth of the range.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The present invention provides oligonucleotides referred to as cyclic structured oligonucleotides (“CSOs”) comprising a functional domain, a cyclizing domain, and a linker segment. Unless noted otherwise, the functional domain and the cyclizing domain are linked at their 5′ ends via a 5′-5′ linkage. Alternatively, in some embodiments, the functional domain and the cyclizing domain are linked at their 3′ ends via a 3′-3′ linkage.

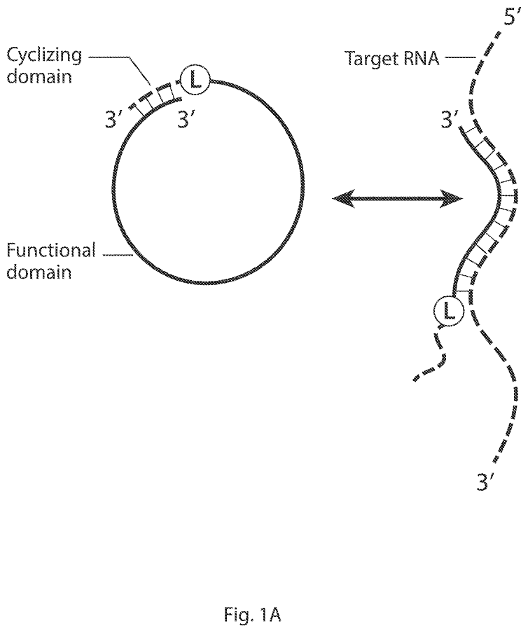

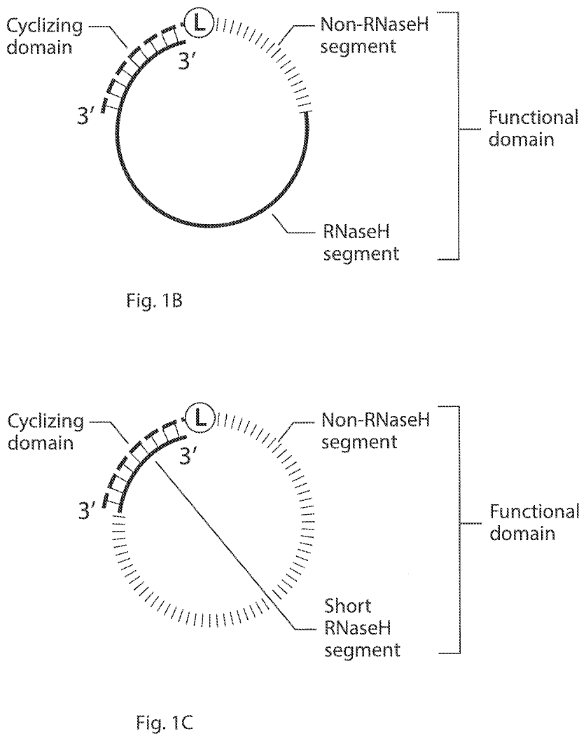

In embodiments, the cyclizing domain is attached to the functional domain on the 5′-end with a 5′-5′ linkage. In this configuration, the cyclizing domain hybridizes with the 3′-end of the oligonucleotide of the functional domain, thereby forming a cyclic structure (e.g., FIG. 1A-FIG. 1C). This design of cyclic oligonucleotides maintains a cyclic form until it is in the presence of and the functional domain hybridizes with a targeted RNA. This structure allows for increased specificity.

In embodiments, the cyclizing domain is attached to the functional domain on the 3′-end with a 3′-3′ linkage. In this configuration, the cyclizing domain hybridizes with the 5′-end of the oligonucleotide of the functional domain, thereby forming a cyclic structure.

The functional domain provides a desired function to the CSO. For example, for gene expression modulation, the oligonucleotide of the functional domain is complementary to a targeted RNA.

In embodiments, the oligonucleotides of the functional domain and the cyclizing domain are DNA or RNA or combinations thereof. In embodiments, the oligonucleotide of the functional domain is DNA or RNA or combinations thereof. In embodiments, the oligonucleotide of the cyclizing domain is DNA or RNA or combinations thereof.

In embodiments, the oligonucleotides of the functional domain and/or the cyclizing domain are unmodified. In embodiments, the oligonucleotides of the functional domain are unmodified. In embodiments, the oligonucleotides of the cyclizing domain are unmodified. In embodiments, the oligonucleotides of the functional domain and the cyclizing domain are unmodified.

In embodiments, at least one nucleotide of the oligonucleotides of the functional domain and/or the cyclizing domain are modified. In embodiments, two or more nucleotides of the oligonucleotides of the functional domain and/or the cyclizing domain are modified.

In embodiments, the oligonucleotide of the functional domain is modified. In embodiments, the oligonucleotide of the functional domain comprises a modification of the inter-nucleotide linkage, sugar, heterocyclic base, or a combination thereof. These modifications could also be appropriately placed at specific positions within the oligonucleotide of the functional domain. Other chemistries and modification are known in the field of oligonucleotides that can be readily used in accordance with the disclosure and are encompassed within the term ‘modified’ as used in the context of an oligonucleotide herein. As used herein, the terms “oligonucleotide of the functional domain” or the “functional domain” are used interchangeably.

In embodiments, the functional domain comprises an oligonucleotide between 15 and 500 nucleotides in length. In embodiments, the oligonucleotide of the functional domain is 17 and 300 nucleotides in length. In embodiments, the oligonucleotide of the functional domain is 17 and 200 nucleotides in length. In embodiments, the oligonucleotide of the functional domain is 17 and 100 nucleotides in length. In embodiments, the oligonucleotide of the functional domain is 17 and 50 nucleotides in length.

In embodiments, the oligonucleotide of the functional domain is 50 and 250 nucleotides in length. In embodiments, the oligonucleotide of the functional domain is 50 and 150 nucleotides in length.

In embodiments, the functional domain comprises an oligonucleotide between 15 and 50 nucleotides in length. In embodiments, the functional domain comprises an oligonucleotide between 17 and 40 nucleotides in length. In embodiments, the functional domain comprises an oligonucleotide between 17 and 25 nucleotides in length. In embodiments, the oligonucleotide of the functional domain is 17 and 22 nucleotides in length.

In embodiments, the oligonucleotide of the functional domain is 17, 18, 19, 20, 21, 22, 23, 24, 25, 26, 27, 28, 29, 30, 31, 32, 33, 34, 35, 36, 37, 38, 39, or 40 nucleotides in length. In embodiments, the functional domain is 17 nucleotides in length. In embodiments, the functional domain is 18 nucleotides in length. In embodiments, the functional domain is 19 nucleotides in length. In embodiments, the functional domain is 20 nucleotides in length. In embodiments, the functional domain is 21 nucleotides in length. In embodiments, the functional domain is 22 nucleotides in length. In embodiments, the functional domain is 23 nucleotides in length. In embodiments, the functional domain is 24 nucleotides in length. In embodiments, the functional domain is 25 nucleotides in length. In embodiments, the functional domain is 26 nucleotides in length. In embodiments, the functional domain is 27 nucleotides in length. In embodiments, the functional domain is 28 nucleotides in length. In embodiments, the functional domain is 29 nucleotides in length. In embodiments, the functional domain is 30 nucleotides in length. In embodiments, the functional domain is 31 nucleotides in length. In embodiments, the functional domain is 32 nucleotides in length. In embodiments, the functional domain is 33 nucleotides in length. In embodiments, the functional domain is 34 nucleotides in length. In embodiments, the functional domain is 35 nucleotides in length. In embodiments, the functional domain is 36 nucleotides in length. In embodiments, the functional domain is 37 nucleotides in length. In embodiments, the functional domain is 38 nucleotides in length. In embodiments, the functional domain is 39 nucleotides in length. In embodiments, the functional domain is 40 nucleotides in length.

In embodiments, the functional domain includes, but is not limited to, an oligonucleotide selected from an antisense oligonucleotide, a microRNA (miRNA), an siRNA, a piRNA, an hnRNA, an ncRNA, an snRNA, an sgRNA, an esiRNA, an shRNA, a lncRNA, a CRISPR-based system, an adenosine deaminase acting on RNA (ADAR) system, or a splicing oligonucleotide. In embodiments, the functional domain includes, but is not limited to, an oligonucleotide selected from an immunostimulatory oligonucleotide or an immune-inhibitory oligonucleotide, also referred to as an immune antagonist oligonucleotide.

The only limitation on the nucleotides and internucleotide linkages of the oligonucleotide of the functional domain is that they do not eliminate (a) the ability of the cyclizing domain to hybridize to and form a duplex with the functional domain of the CSO under the desired conditions, and (b) the ability of the functional domain to carry out its intended function (e.g., in the case of a functional domain that is an antisense oligonucleotide, to hybridize to and form a duplex with a complementary RNA segment under physiological conditions, which duplex is a substrate for RNase H). Preferred nucleotides and internucleotide linkages are those that will enhance the stability of the CSO to nucleases and other forms of chemical degradation and/or enhance the ability of the functional domain to carry out its intended function.

In embodiments, the internucleotide linkages of the functional domain are phosphorothioate internucleotide linkages, phosphodiester internucleotide linkages or a combination thereof.

The oligonucleotide of cyclizing domain is complementary to a sequence of nucleotides within the functional domain and of polarity opposite to the sequence of nucleotides in the functional domain to which it is complementary.

In embodiments, the oligonucleotide of the cyclizing domain is modified. In embodiments, the oligonucleotide of the cyclizing domain comprises a modification of the inter-nucleotide linkage, sugar, heterocyclic base, or a combination thereof. These modifications could also be placed at specific positions within the oligonucleotide of the cyclizing domain. As used herein, the terms “oligonucleotide of the cyclizing domain” or the “cyclizing domain” are used interchangeably.

In embodiments, the internucleotide linkages of the cyclizing domain are phosphorothioate internucleotide linkages, phosphodiester internucleotide linkages or a combination thereof.

In embodiments, the cyclizing domain comprises an oligonucleotide between 4 and 400 nucleotides in length. In embodiments, the cyclizing domain comprises an oligonucleotide between 4 and 100 nucleotides in length. In embodiments, the cyclizing domain comprises an oligonucleotide between 4 and 75 nucleotides in length. In embodiments, the cyclizing domain comprises an oligonucleotide between 4 and 50 nucleotides in length. In embodiments, the cyclizing domain comprises an oligonucleotide between 4 and 40 nucleotides in length. In embodiments, the cyclizing domain comprises an oligonucleotide between 4 and 30 nucleotides in length. In embodiments, the cyclizing domain comprises an oligonucleotide between 4 and 25 nucleotides in length. In embodiments, the cyclizing domain comprises an oligonucleotide between 4 and 12 nucleotides in length. In embodiments, the cyclizing domain comprises an oligonucleotide between 4 and 10 nucleotides in length. In embodiments, the cyclizing domain comprises an oligonucleotide between 4 and 6 nucleotides in length.

In embodiments, the cyclizing domain comprises an oligonucleotide between 5 and 8 nucleotides in length. In embodiments, the oligonucleotide of the cyclizing domain is 4, 5, 6, 7, 8, 9 or 10 nucleotides in length. In embodiments, the oligonucleotide of the cyclizing domain is 4 nucleotides in length. In embodiments, the oligonucleotide of the cyclizing domain is 5 nucleotides in length. In embodiments, the oligonucleotide of the cyclizing domain is 6 nucleotides in length. In embodiments, the oligonucleotide of the cyclizing domain is 7 nucleotides in length. In embodiments, the oligonucleotide of the cyclizing domain is 8 nucleotides in length. In embodiments, the oligonucleotide of the cyclizing domain is 9 nucleotides in length. In embodiments, the oligonucleotide of the cyclizing domain is 10 nucleotides in length.

In embodiments, the cyclizing domain comprises an oligonucleotide between 15 and 40 nucleotides in length. In embodiments, the cyclizing domain comprises an oligonucleotide between 17 and 30 nucleotides in length. In embodiments, the oligonucleotide of the cyclizing domain is 15, 16, 17, 18, 19, 20, 21, 22, 23, 24, 25, 26, 27, 28, 29, 30, 31, 32, 33, 34, 35, 36, 37, 38, 39, or 40 nucleotides in length. In embodiments, the oligonucleotide of the cyclizing domain is 15 nucleotides in length. In embodiments, the oligonucleotide of the cyclizing domain is 16 nucleotides in length. In embodiments, the oligonucleotide of the cyclizing domain is 17 nucleotides in length. In embodiments, the oligonucleotide of the cyclizing domain is 18 nucleotides in length. In embodiments, the oligonucleotide of the cyclizing domain is 19 nucleotides in length. In embodiments, the oligonucleotide of the cyclizing domain is 20 nucleotides in length. In embodiments, the oligonucleotide of the cyclizing domain is 21 nucleotides in length. In embodiments, the oligonucleotide of the cyclizing domain is 22 nucleotides in length. In embodiments, the oligonucleotide of the cyclizing domain is 23 nucleotides in length. In embodiments, the oligonucleotide of the cyclizing domain is 24 nucleotides in length. In embodiments, the oligonucleotide of the cyclizing domain is 25 nucleotides in length. In embodiments, the oligonucleotide of the cyclizing domain is 26 nucleotides in length. In embodiments, the oligonucleotide of the cyclizing domain is 27 nucleotides in length. In embodiments, the oligonucleotide of the cyclizing domain is 28 nucleotides in length. In embodiments, the oligonucleotide of the cyclizing domain is 29 nucleotides in length. In embodiments, the oligonucleotide of the cyclizing domain is 30 nucleotides in length. In embodiments, the oligonucleotide of the cyclizing domain is 31 nucleotides in length. In embodiments, the oligonucleotide of the cyclizing domain is 32 nucleotides in length. In embodiments, the oligonucleotide of the cyclizing domain is 33 nucleotides in length. In embodiments, the oligonucleotide of the cyclizing domain is 34 nucleotides in length. In embodiments, the oligonucleotide of the cyclizing domain is 35 nucleotides in length. In embodiments, the oligonucleotide of the cyclizing domain is 36 nucleotides in length. In embodiments, the oligonucleotide of the cyclizing domain is 37 nucleotides in length. In embodiments, the oligonucleotide of the cyclizing domain is 38 nucleotides in length. In embodiments, the oligonucleotide of the cyclizing domain is 39 nucleotides in length. In embodiments, the oligonucleotide of the cyclizing domain is 40 nucleotides in length.

As used herein, the term “polarity” refers to the concept of directionality in primary structure (e.g., 3′→5′ and 5′→3′ in the case of DNA and RNA, or N-terminal→C-terminal (or vice versa) in the case of PNAs). In the case where the CSOs of the invention comprise oligonucleotides, for example, which hybridize by Watson-Crick base pairing in anti-parallel fashion, the cyclizing domain will be in the 5′→3′ (or 2′) configuration and the sequence of nucleotides to which it is complementary in the functional domain will be in the 3′ (or 2′)→5′ configuration. The change in polarity in the CSO can occur anywhere in the CSO other than in the cyclizing domain and the sequence of nucleotides in the functional domain to which the cyclizing domain is complementary. In a preferred embodiment where the CSO comprises oligonucleotides, the functional domain is in the 3′→5′ configuration and the cyclizing domain is in the 5′→3′ configuration, such that the functional domain and the cyclizing domain are linked via a 5′-5′ linkage. In some embodiments, the functional domain is in the 5′→3′ configuration and the cyclizing domain is in the 3′→5′ configuration, such that the functional domain and the cyclizing domain are linked via a 3′-3′ linkage.

In embodiments, the functional domain and the cyclizing domain are covalently linked to each other through the linker segment. In embodiments, the linker segment is a direct bond, a nucleotide or oligonucleotide between 2 and 5 nucleotides in length, or other chemical moiety, or combinations thereof. In some embodiments, the linker segment can be cleavable.

The only limitation on the linker segment is that it does not eliminate the essential functions of the CSO, namely (a) the ability of the CSO to form an intramolecular cyclic structure under the conditions of interest (e.g., physiological conditions) and (b) the ability of the functional domain to carry out its intended function. Preferred “other chemical moiety” linkers include, but are not limited to, C₂-C₆ alkyl, ethylene glycol, tri (ethylene glycol), tetra (ethylene glycol), penta (ethylene glycol), hexa (ethylene glycol) and —NH(CH₂)ₙNH—, wherein n is 2, 3, 4, 5, or 6. Alternatively, the linker segment can be a combination of the foregoing. In a preferred embodiment, the linker is a direct bond, in which case the functional and cyclizing domains are directly bound. In an embodiment, the linker is ethylene glycol. In embodiments, the linker is a C₂-C₆ alkyl. In embodiments, the linker is a C₂ alkyl. In embodiments, the linker is a C₃ alkyl. In embodiments, the linker is a C₄ alkyl. In embodiments, the linker is a C₅ alkyl. In embodiments, the linker is a C₆ alkyl.

The oligonucleotide of the functional domain has a terminal end and a linker end. As the name implies, the linker end is the end of the oligonucleotide linked to the cyclizing domain through the linker segment. In general, the CSO is constructed so the terminal end of the functional domain will form a duplex with the cyclizing domain, i.e., the cyclizing domain is complementary to the terminal end of the functional domain.

As used herein, the term “complementary” refers to a pair of nucleobases (or simply a “base”) that hydrogen bond to each other in preference to other heterocyclic bases under selected (e.g., physiological) conditions (or some degree of complementarity thereof as context may require in the instance of assessing “complementary-ness” of oligonucleotides). When the nucleobases are modified or unmodified, natural or synthetic purines and pyrimidines, the term “complementary” means complementary in the Watson Crick sense.

When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mismatched” and the strand is considered partially complementary. Accordingly, strands can be varying degrees of partially complementary (e.g., 0%<x<100% complementary), until no bases align, at which point they are non-complementary (e.g., 0% complementary). As is readily understood and recognized by one of skill in the art, full (i.e., complete, 100%) complementarity is not required for hybridization of strands of nucleic acids (e.g., oligonucleotides, antisense or otherwise).

In embodiments, the target RNA may be an mRNA, pre-mRNA, ncRNA, lncRNA, or microRNA. In embodiments, the target RNA is mRNA.

In embodiments, cyclic structured oligonucleotide according to the invention is part of a pharmaceutical composition comprising a pharmaceutically acceptable carrier.

The pharmaceutical composition comprising the cyclic structured oligonucleotide of the invention may further comprise any other agent or therapy useful for treating or preventing a disease or condition and does not diminish the gene expression modulation effect of the cyclic structured oligonucleotide according to the invention. Agent(s) useful for treating or preventing the disease or condition includes, but is not limited to, small molecules, peptides, vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the cyclic structured oligonucleotide according to the invention can be administered in combination with other compounds (for example formulated with lipids or liposomes, and conjugated to peptides, antibodies, or small molecules) to enhance the specificity or magnitude of the gene expression modulation of the cyclic structured oligonucleotide according to the invention.

In embodiments, the oligonucleotide of the functional domain comprises at least one phosphorothioate internucleotide linkage. In embodiments, at least half of the internucleotide linkages are phosphorothioate. In embodiments, all of the internucleotide linkages are phosphorothioate.

In embodiments, the oligonucleotide of the functional domain is single-stranded.

In embodiments, the oligonucleotide of the functional domain is at least 90% complementary over its entire length to a portion of a target RNA. In embodiments, the oligonucleotide of the functional domain is at least 95% complementary over its entire length to a portion of the target RNA. In embodiments, the oligonucleotide of the functional domain is at least 97% complementary over its entire length to a portion of the target RNA. In embodiments, the oligonucleotide of the functional domain is at least 98% complementary over its entire length to a portion of the target RNA. In embodiments, the oligonucleotide of the functional domain is at least 99% complementary over its entire length to a portion of the target RNA. In embodiments, the oligonucleotide of the functional domain is at least 100% complementary over its entire length to a portion of the target RNA.

In embodiments, wherein the oligonucleotide of the functional domain is an oligonucleotide of a CRISPR-based system or an adenosine deaminase acting on RNA (ADAR) system, then the portion of the oligonucleotide that is complementary to the target RNA (complementary domain) is at least 90% complementary over its entire length to a portion of the target RNA, preferably at least 95% complementary, preferably at least 97% complementary, preferably at least 98% complementary, preferably at least 99% complementary, or preferably at least 100% complementary.

In any of the compounds, compositions, or methods described herein, the CSO of the invention is not an oligonucleotides having the sequence 3′-CGGTCACTCCTCCGTGCG-5′-5′-GCCAGT-3′ (SEQ ID 218), 3′-CGGTCACTCCTCCGTGCG-5′-5′-GCGAAT-3′ (SEQ ID 218), 5′-GCGTGCCTCCTCACTGGC-3′-3′-CGCACG-5′ (SEQ ID 218), 5′-GCGTGCCTCCTCACGGC-3′-3′-GGAACC-5′ (SEQ ID 219), 5′-GCGTGCCTCCTCACTGGC-3′-3′-CGCAC-5′ (SEQ ID 218), 5′-GCGTGCCTCCTCACTGGC-3′-3′-GGAACCG-5′ (SEQ ID 218), 5′-GCGTGCCTCCTCACTGGC-3′-3′-CGCACGGA-5′ (SEQ ID 218), 5′-GCGTGCCTCCTCACTGGC-3′-3′-GGAAC-5′ (SEQ ID 218), or 5′-GCGTGCCTCCTCACTGGC-3′-3′-GGAACAG-5′ (SEQ ID 218).

### Functional Domains

In various embodiments the functional domain of the CSO is an oligonucleotide as further described below. The cyclizing domain and the linker of the CSO is as described above unless otherwise noted.

### Inhibition of Gene Expression

In embodiments, the invention provides a cyclic structured oligonucleotide (CSO) comprising a functional domain, a cyclizing domain, and a linker, wherein the functional domain and the cyclizing domain are linked at their 5′ ends, wherein the functional domain comprises an oligonucleotide between 15 and 45 nucleotides in length and is complementary to targeted RNA; wherein the cyclizing domain comprises an oligonucleotide between 4 and 12 nucleotides in length and is complementary to a sequence of nucleotides within the functional domain and of polarity opposite to the sequence of nucleotides in the functional domain to which it is complementary; wherein functional domain comprises a gene modulating oligonucleotide. In embodiments, the oligonucleotide of the functional domain is modified.

In embodiments, the gene modulating oligonucleotide (i.e., an oligonucleotide that can modulate the expression of a target gene) of the functional domain includes, but is not limited to, an antisense oligonucleotide, a microRNA (miRNA), a piRNA, a hnRNA, a ncRNA, a snRNA, a sgRNA, an esiRNA, an shRNA, or a lncRNA.

In embodiments, the modification of the antisense oligonucleotide comprises at least one modified nucleobase, sugar and/or internucleotide linkage.

As shown herein, the CSOs of the invention comprising an antisense oligonucleotide linked to a cyclizing domain via 5′-5′ linkage surprisingly demonstrated increased potency. This observation was in complete contrast to earlier hypotheses that increased stability (e.g., 3′-3′ linked oligonucleotides) would translate to increased potency. Furthermore, the lack of a 5′-end would make antisense oligonucleotides of the CSO less inflammatory. This design permits antisense oligonucleotides to unfold to linear structure and to be active in cells where the target RNA is expressed.

In the case where the CSO is an antisense oligonucleotide, it is in cyclic form until it is in the presence of complementary target RNA where it adopts a linear form and binds to the target RNA. The changes from cyclic form to linear form could be confirmed by thermal melting and RNase H cleavage studies. In the linear form, the functional domain hybridizes (under physiological conditions, at a minimum) with the complementary target RNA to form a duplex. This duplex is a substrate for RNase H, and, in the presence of RNase H and under the proper conditions (e.g., physiological), the RNA strand of the duplex will be cleaved by the RNase H, thereby preventing expression.

CSOs comprising antisense functional domains maintain activity in cell cultures. The advantage foreseen with these CSOs is that their formation of intramolecular cyclic structures allows for less interaction with non-targeted macromolecules (including nucleic acids and proteins), have reduced polyanionic-related side effects, and will linearize in the presence of the targeted gene or RNA only. Additionally, due to cyclic structure, these CSOs can escape from endosomes due to a lack of interactions of 5′-end.

The oligonucleotides of the invention are isolated oligonucleotides. The term “isolated” means altered or removed from the natural state through human intervention. For example, an oligonucleotide naturally present in a living animal is not “isolated,” but a synthetic oligonucleotide, or an oligonucleotide partially or completely separated from the coexisting materials of its natural state is “isolated.” An isolated oligonucleotide can exist in substantially purified form, or can exist in a non-native environment such as, for example, a cell into which the oligonucleotide has been delivered. The oligonucleotides of the invention can comprise partially purified DNA and/or RNA, substantially pure DNA and/or RNA, synthetic DNA and/or RNA, or recombinantly produced DNA and/or RNA, as well as altered DNA and/or RNA that differs from naturally occurring DNA and/or RNA by the addition, deletion, substitution and/or alteration of one or more nucleotides. Such alterations can include addition of non-nucleotide material, such as to the end(s) of the oligonucleotide or to one or more internal nucleotides of the oligonucleotide, including modifications that make the oligonucleotide resistant to nuclease digestion.

The terms “microRNA,” “miRNA,” and “MiR” are interchangeable and refer to endogenous or artificial non-coding RNAs that can regulate gene expression. It is believed that miRNAs function via RNA interference. The design of such microRNAs is within the skill of ordinary artisans.

The terms “piRNA” and “Piwi-interacting RNA” are interchangeable and refer to a class of small RNAs involved in gene silencing. PiRNA molecules typically are between 26 and 31 nucleotides in length. The design of such PiRNAs is within the skill of ordinary artisans.

In embodiments, the antisense oligonucleotide of the functional domain is as described in WO2020/191177, which is incorporated herein by reference in its entirety. In embodiments, the antisense oligonucleotide of the functional domain is a modified oligonucleotide comprising or consisting of an antisense oligonucleotide compound 17 to 25 nucleotides in length, wherein the antisense oligonucleotide compound comprises a 3′ domain and a 5′ domain, wherein the 3′ domain is 10 to 12 nucleotides in length and each nucleotide comprises a deoxyribonucleotide and a phosphodiester or phosphothioate internucleotide linkage or combinations thereof; and wherein the 5′ domain is 5 to 15 nucleotides in length, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone. Such antisense oligonucleotides are referred to as “splitmer”.

In embodiments, the splitmer of the functional domain comprises 17 to 25 linked nucleotides having at least 12 contiguous nucleobases complementary to an equal length portion of a target RNA.

In embodiments, the modified ribonucleotides of the splitmer comprise 2′-substituted nucleotides are as described herein. In embodiments, the 2′-substituted nucleotides are selected from 2′ O-methyl ribonucleotides or 2′-MOE.

In some embodiments, the 3′ domain comprises nucleotides at positions 1, 2, 3, 4, 5, 6, 7, 8, 9, 10, 11, and 12 from the 3′ end. In some embodiments, the 3′ domain comprises nucleotides at positions 1, 2, 3, 4, 5, 6, 7, 8, 9, 10, and 11 from the 3′ end. In some embodiments, the 3′ domain comprises nucleotides at positions 1, 2, 3, 4, 5, 6, 7, 8, 9, and 10 from the 3′ end.

In some embodiments, when the 3′ domain of the antisense oligonucleotide is 12 nucleotides in length, the antisense oligonucleotides of the invention are represented by Formula (I):

5′-NₘN₁₄N₁₃N₁₂N₁₁N₁₀N₉N₈N₇N₆N₅N₄N₃N₂N₁-3′

wherein
N is any nucleotide;
N₁₃ through Nₘ comprises the 5′ domain;
N₁ through N₁₂ comprises the 3′ domain; and
m is selected from 0, 1, 2, 3, 4, 5, 6, 7, 8, 9, 10, or 11.

In some embodiments, when the 3′ domain of the antisense oligonucleotide is 11 nucleotides in length, the antisense oligonucleotides of the invention are represented by Formula (Ia):

5′-NₘN₁₄N₁₃N₁₂N₁₁N₁₀N₉N₈N₇N₆N₅N₄N₃N₂N₁-3′

wherein
N is any nucleotide;
N₁₂ through Nₘ comprises the 5′ domain;
N₁ through N₁₁ comprises the 3′ domain; and
m is selected from 0, 1, 2, 3, 4, 5, 6, 7, 8, 9, 10, or 11.

In some embodiments, the antisense oligonucleotides of the invention are represented by Formula (Ib):

5′-NₘN₁₄N₁₃N₁₂N₁₁N₁₀N₉N₈N₇N₆N₅N₄N₃N₂N₁-3′

wherein
N is any nucleotide;
N₁₁ through Nₘ comprises the 5′ domain;
N₁ through N₁₀ comprises the 3′ domain; and
m is selected from 0, 1, 2, 3, 4, 5, 6, 7, 8, 9, 10, or 11.

In some embodiments, m is 0. In some embodiments, m is selected from 1, 2, 3, 4, 5, 6, or 7. In some embodiments, m is selected from 1, 2, 3, 4, 5, or 6. In some embodiments, m is selected from 1, 2, 3, 4, or 5. In some embodiments, m is selected from 1, 2, 3, or 4. In some embodiments, m is selected from 1, 2, or 3. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11.

In embodiments, the antisense oligonucleotide compound of the functional domain is 17 to 25 nucleotides in length comprising at least 12 contiguous nucleobases complementary to an equal length portion of a target RNA sequence, wherein the antisense oligonucleotide compound comprises a 3′ domain and a 5′ domain, which is contiguous with the 3′ domain, wherein the 3′ domain is 10 to 12 nucleotides in length and each nucleotide comprises a deoxyribonucleotide and a phosphodiester or phosphothioate internucleotide linkage or combinations thereof; and wherein the 5′ domain is 5 to 15 nucleotides in length, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone.

In embodiments, the 3′ domain is 12 nucleotides in length and comprises nucleotides at positions 1, 2, 3, 4, 5, 6, 7, 8, 9, 10, 11, and 12 from the 3′ end (position 1 is the 3′ end). In embodiments, the 3′ domain is 11 nucleotides in length and comprises nucleotides at positions 1, 2, 3, 4, 5, 6, 7, 8, 9, 10, and 11 from the 3′ end. In embodiments, the 3′ domain is 12 nucleotides in length and comprises nucleotides at positions 1, 2, 3, 4, 5, 6, 7, 8, 9, and 10 from the 3′ end.

In embodiments, the nucleotides of the 3′ domain comprise a natural nucleobase. In some embodiments, the nucleobases and sugars of the nucleotides of the 3′ domain of the antisense oligonucleotide according to the invention are unmodified. In this respect, the nucleobases and sugars of the nucleotides of the 3′ domain of the antisense oligonucleotide according to the invention are naturally occurring. Each of the nucleotides of the 3′ domain comprise a deoxyribonucleotide and a phosphodiester or phosphorothioate internucleotide linkage or combinations thereof. The nucleotides of the 3′-domain comprise natural deoxyribose sugar and phosphorothioate, phosphodiester or other phosphorus-based linkages or combinations thereof, which are known to activate RNase H.

In embodiments, at least one of the nucleotides of the 3′ domain comprises a modified nucleobase.

In embodiments, the nucleotides at the 9^{th} or 10^{th} positions from the 3′ end are not modified. In embodiments, the nucleotide at the 11^{th} position from the 3′ end is not modified.

In embodiments, the oligonucleotide comprises at least one phosphorothioate internucleotide linkage.

In embodiments, at least half of the internucleotide linkages are phosphorothioate.

In embodiments, the antisense oligonucleotide is single stranded.

As used here, the term “5′ domain” refers to the nucleotides beginning at the first nucleotide following the 3′ domain and goes to the 5′ end. The 5′ domain hybridizes to the target RNA but does not allow RNase H to excise the target RNA in this domain. The term “5′ domain” is generally 2 to 15 nucleotides in length and refers to the 11^{th} through the 25^{th} nucleotides (the 1^{st} nucleotide is the 3′ end), 12^{th} through the 25^{th} nucleotides, or 13^{th} through the 25^{th} nucleotides of the antisense oligonucleotide as measured from the 3′ end depending on the length of the 3′ domain.

For example, an antisense oligonucleotide compound that is 17 nucleotides in length may comprise a 3′ domain from position 1 to position 10 and a 5′ domain from position 11 to position 17. The designation of the modified nucleotide is position-specific, as opposed to nucleotide-specific.

The 5′ domain comprises nucleotides having non-RNase H activating modifications such as modified sugars and/or modified backbones, which do not activate RNase H. In some embodiments, the 5′ domain comprises nucleotides comprising a modified sugar. In some embodiments, the 5′ domain comprises nucleotides comprising a modified backbone. In some embodiments, the 5′ domain comprises nucleotides comprising both a modified sugar and modified backbone. In embodiments, the modified backbone is a non-phosphorus-based backbone.

This design of antisense allows for targeted RNA cleavage at the specific sites towards the 5′ end of 3′ domain.

In any of these embodiments it is contemplated that the 5′ domain comprises at least one nucleotide having a backbone modification or substitution and/or a sugar modification or substitution. In some embodiments, a nucleotide at one position within the 5′ domain, at some of the positions within the 5′ domain, or at all positions within the 5′ domain comprises a backbone modification or substitution and/or a sugar modification or substitution. In one embodiment, the 5′ domain comprises one nucleotide comprising a modified backbone and/or sugar. In one embodiment, the 5′ domain comprises at least two nucleotides comprising a modified backbone and/or sugar. In one embodiment, the 5′ domain comprises at least three nucleotides comprising a modified backbone and/or sugar. In one embodiment, the 5′ domain comprises at least four nucleotides comprising a modified backbone and/or sugar. In one embodiment, the 5′ domain comprises at least five nucleotides comprising a modified backbone and/or sugar. In one embodiment, the 5′ domain comprises at least six nucleotides comprising a modified backbone and/or sugar. In one embodiment, the 5′ domain comprises at least seven nucleotides comprising a modified backbone and/or sugar. In one embodiment, the 5′ domain comprises at least eight nucleotides comprising a modified backbone and/or sugar. In one embodiment, all of the nucleotides of the 5′ domain are nucleotides comprising a modified backbone and/or sugar.

It is specifically contemplated that embodiments discussed herein, in the context of a specific nucleotide and position, may be implemented with respect to a position relative to the 3′ end. For example, an antisense oligonucleotide with a modified nucleotide at position 13 refers to an antisense oligonucleotide having a modified nucleotide at position 13 from the 3′ end of the antisense oligonucleotide.

In embodiments, the antisense oligonucleotide of the functional domain is at least 90% complementary over its entire length to a portion of the target RNA.

In embodiments, the oligonucleotide of the functional domain is a “gapmer”. As used herein, a gapmers is a chimeric antisense oligonucleotide that contains a central block of deoxynucleotide monomers sufficiently long to induce RNase H cleavage. Usually, the gapmers of the invention are directed against one or more mRNA encoding a target mRNA. The design of such gapmers is within the skill of ordinary artisans.

In embodiments, the antisense oligonucleotide of the functional domain is a modified oligonucleotides comprising or consisting of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer independently comprise 1-6 nucleosides. In certain embodiments, the wings of a gapmer independently comprise 1-5 nucleosides. In certain embodiments, the wings of a gapmer comprise the same number of nucleosides. In certain embodiments, the wings of a gapmer comprise 4 nucleosides. In certain embodiments, each nucleoside of each wing of a gapmer is a modified nucleoside.

In certain embodiments, the gap of a gapmer comprises 7-24 nucleosides. In certain embodiments, the gap of a gapmer comprises 7-18 nucleosides. In certain embodiments, the gap of a gapmer comprises 9-14 nucleosides. In certain embodiments, the gap of a gapmer comprises 7-23 nucleosides. In certain embodiments, the gap of a gapmer comprises 9 nucleosides. In certain embodiments, the gap of a gapmer comprises 10 nucleosides. In certain embodiments, the gap of a gapmer comprises 11 nucleosides. In certain embodiments, the gap of a gapmer comprises 13 nucleosides. In certain embodiments, the gap of a gapmer comprises 14 nucleosides. In certain embodiments, the gap of a gapmer comprises 17 nucleosides. In certain embodiments, the gap of a gapmer comprises 18 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in the wings and the gap nucleosides comprise unmodified deoxynucleosides sugars. Thus, a 5-11-5 MOE or OMe gapmer consists of 5 linked MOE or OMe modified nucleosides in the 5′-wing, 11 linked deoxynucleosides in the gap, and 5 linked MOE or OMe nucleosides in the 3′-wing.

In certain embodiments, modified oligonucleotides are 4-13-4 MOE or OMe gapmers. In certain embodiments, modified oligonucleotides are 5-11-5 MOE or OME gapmers. In certain embodiments, modified oligonucleotides are 3-15-3 BNA gapmers. In certain embodiments, modified oligonucleotides are 3-15-3 LNA gapmers.

In any of the embodiments described herein, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification. In certain embodiments, the uniformly modified sugar motif is 12 to 30 nucleosides in length. In certain embodiments, each nucleoside of the uniformly modified sugar motif is a 2′-substituted nucleoside, a sugar surrogate, or a bicyclic nucleoside. In certain embodiments, each nucleoside of the uniformly modified sugar motif comprises either a 2′-OCH₂CH₂OCH₃ group or a 2′-OCH₃ group. In certain embodiments, modified oligonucleotides having at least one fully modified sugar motif may also have at least 1, at least 2, at least 3, or at least 4 2′-deoxynucleosides.

Examples of cyclic structured oligonucleotides useful for gene silencing include, but are not limited to, the cyclic structured oligonucleotides of Table 1. Oligonucleotide #s 1-6 in Table 1 are targeted to proprotein convertase subtilisin/kexin type 9 (PCSK9). Oligonucleotide #s 1512-1520, 1536-1545 and 1605 in Table 1 are targeted to patatin-like phospholipase domain-containing protein 3 (PNPLA3). Cyclic structured oligonucleotides with functional domains directed to any other target of interest are well within the skill of one in the art. All internucleotide linkages are phosphorothioate linkages unless otherwise noted.

TABLE 1

| Compound | | |
|---|---|---|
| # | SEQ ID NO(s) | Sequence |
| 1 | SEQ ID NO: 146 | 3′-T₂C₂G₂T₂G₂G-5′-5′-GGTCTCCTCCATCAGCACC-3′ |
| | & SEQ ID NO: 1 | |
| 2 | SEQ ID NO: 147 | 3′-T₂C₂G₂T₂G₂G₂-5′-5′-G₁G₁U₁CTCCTCCATCAGCA₁C₁C₁-3′ |
| | & SEQ ID NO: 2 | |
| 3 | SEQ ID NO: 147 | 3′-T₂C₂G₂T₂G₂G₂-5′-5′-G₁G₁U₁C₁U₁C₁CTCCATCAGCACC-3′ |
| | & SEQ ID NO: 3 | |
| 4 | SEQ ID NO: 4 | 5′-GGTCTCCTCCATCAGCACC-3′ |
| 5 | SEQ ID NO: 5 & | 5′-GGTCTCCTCCATCAGCACC-3′-3′-C₂C₂A₂G₂A₂G-5′ |
| | SEQ ID NO: 148 | |
| 6 | SEQ ID NO: 6 & | 3′-CCACGACTACCTCCTCTGG-5′-5′- |
| | SEQ ID NO: 6 | GGTCTCCTCCATCAGCACC-3′ |
| 1605 | SEQ ID NO: 59 & | 3′-GACGAAGTAGGGGAAGATG-5′-5′-CTGCTTC-3′ |
| | SEQ ID NO: 149 | |
| 1512 | SEQ ID NO: 61 & | 3′-GACGAAGTAGGGGAAGATG-5′-5′-CTGCTT-3′ |
| | SEQ ID NO: 150 | |
| 1513 | SEQ ID NO: 62 & | 3′-GACGAAGTAGGGGAAGATG-5′-5′-CTGCT-3′ |
| | SEQ ID NO: 151 | |
| 1514 | SEQ ID NO: 63 & | 3′-GACGAAGTAGGGGAAGATG-5′-5′-CTGC-3′ |
| | SEQ ID NO: 152 | |
| 1515 | SEQ ID NO: 64 & | 3′-GACGAAGTAGGGGAAGATG-5′-5′-C₂T₂G₂C₂T₂T₂-3′ |
| | SEQ ID NO: 153 | |
| 1516 | SEQ ID NO: 65 & | 3′-GACGAAGTAGGGGAAGATG-5′-5′-C₂T₂G₂C₂T₂-3′ |
| | SEQ ID NO: 154 | |
| 1517 | SEQ ID NO: 66 & | 3′-GACGAAGTAGG₁G₁G₁A₁A₁G₁A₁T₁G₁-5′-5′-C₂T₂G₂C₂T₂T₂-3′ |
| | SEQ ID NO: 153 | |
| 1518 | SEQ ID NO: 67 & | 3′-GACGAAGTAGG₁G₁G₁A₁A₁G₁A₁T₁G₁-5′-5′-C₂T₂G₂C₂T₂-3′ |
| | SEQ ID NO: 154 | |
| 1519 | SEQ ID NO: 68 & | 3′-GACGAAGTAGGGG₁A₁A₁G₁A₁T₁G₁-5′-5′-C₂T₂G₂C₂T₂T₂-3′ |
| | SEQ ID NO: 153 | |
| 1520 | SEQ ID NO: 69 & | 3′-GACGAAGTAGGGG₁A₁A₁G₁A₁T₁G₁-5′-5′-C₂T₂G₂C₂T₂-3′ |
| | SEQ ID NO: 154 | |
| 1536 | SEQ ID NO :. 70 & | 3′-GACGAAGTACGGGAAGATG-5′-5′-CTGCTTC-3′ |
| | SEQ ID NO: 149 | |
| 1537 | SEQ ID NO: 71 & | 3′-GACGAAGTACGGGAAGATG-5′-5′-CTGCTT-3′ |
| | SEQ ID NO: 150 | |
| 1538 | SEQ ID NO: 72 & | 3′-GACGAAGTACGGGAAGATG-5′-5′-CTGCT-3′ |
| | SEQ ID NO: 151 | |
| 1539 | SEQ ID NO: 73 & | 3′-GACGAAGTACGGGAAGATG-5′-5′-CTGC-3′ |
| | SEQ ID NO: 152 | |
| 1540 | SEQ ID NO: 74 & | 3′-GACGAAGTACGGGAAGATG-5′-5′-C2T2G2C2T2T2-3′ |
| | SEQ ID NO: 153 | |
| 1541 | SEQ ID NO: 75 & | 3′-GACGAAGTACGGGAAGATG-5′-5′-C2T2G2C2T2-3′ |
| | SEQ ID NO: 154 | |
| 1542 | SEQ ID NO: 76 & | 3′-GACGAAGTACG₁G₁G₁A₁A₁G₁A₁T₁G₁-5′-5′-C₂T₂G₂C₂T₂T₂-3′ |
| | SEQ ID NO: 153 | |
| 1543 | SEQ ID NO: 77 & | 3′-GACGAAGTACG₁G₁G₁A₁A₁G₁A₁T₁G₁-5′-5′-C₂T₂G₂C₂T₂-3′ |
| | SEQ ID NO: 154 | |
| 1544 | SEQ ID NO: 78 & | 3′-GACGAAGTACGG₁G₁A₁A₁G₁A₁T₁G₁-5′-5′-C₂T₂G₂C₂T₂T₂-3′ |
| | SEQ ID NO: 153 | |
| 1545 | SEQ ID NO: 79 & | 3′-GACGAAGTACGG₁G₁A₁A₁G₁A₁T₁G₁-5′-5′-C₂T₂G₂C₂T₂-3′ |
| | SEQ ID NO: 154 | |
| 1546 | SEQ ID NO: 130 & | 3′-GACGAAGTAGG₁G₁G₁A₁A₁G₁A₁T₁G₁-5′-5′-CTGCTT-3′ |
| | SEQ ID NO: 150 | |
| 1547 | SEQ ID NO: 131 & | 3′-GACGAAGTAGG₁G₁G₁A₁A₁G₁A₁T₁G₁-5′-5′-CTGCT-3′ |
| | SEQ ID NO: 151 | |
| 1548 | SEQ ID NO: 132 & | 3′-GACGAAGTAGGGG₁A₁A₁G₁A₁T₁G₁-5′-5′-CTGCTT-3′ |
| | SEQ ID NO: 150 | |
| 1549 | SEQ ID NO: 133 & | 3′-GACGAAGTAGGG₁G₁A₁A₁G₁A₁T₁G₁-5′-5′-CTGCT-3′ |
| | SEQ ID NO: 151 | |
| 1550 | SEQ ID NO: 134 & | 3′-GACGAAGTACG₁G₁G₁A₁A₁G₁A₁T₁G₁-5′-5′-CTGCTT-3′ |
| | SEQ ID NO: 150 | |
| 1551 | SEQ ID NO: 135 & | 3′-GACGAAGTACG₁G₁G₁A₁A₁G₁A₁T₁G₁-5′-5′-CTGCT-3′ |
| | SEQ ID NO: 151 | |
| 1552 | SEQ ID NO: 136 & | 3′-GACGAAGTACGG₁G₁A₁A₁G₁A₁T₁G₁-5′-5′-CTGCTT-3′ |
| | SEQ ID NO: 150 | |
| 1553 | SEQ ID NO: 137 & | 3′-GACGAAGTACGG₁G₁A₁A₁G₁A₁T₁G₁-5′-5′-CTGCT-3′ |
| | SEQ ID NO: 151 | |
| 1554 | SEQ ID NO: 138 & | 3′-GACGAAGTAGG₁G₁G₁A₁A₁G₁A₁T₁G₁-5′-5′-CT₂GC₂TT₂-3′ |
| | SEQ ID NO: 156 | |
| 1555 | SEQ ID NO: 139 & | 3′-GACGAAGTAGG₁G₁G₁A₁A₁G₁A₁T₁G₁-5′-5′-CT₂GC₂T-3′ |
| | SEQ ID NO: 157 | |
| 1556 | SEQ ID NO: 140 & | 3′-GACGAAGTAGGGG₁A₁A₁G₁A₁T₁G₁-5′-5′-CT₂GC₂TT₂-3′ |
| | SEQ ID NO: 156 | |
| 1557 | SEQ ID NO: 141 & | 3′-GACGAAGTAGGGG₁A₁A₁G₁A₁T₁G₁-5′-5′-CT₂GC₂T-3′ |
| | SEQ ID NO: 157 | |
| 1558 | SEQ ID NO: 142 & | 3′-GACGAAGTACG₁G₁G₁A₁A₁G₁A₁T₁G₁-5′-5′-CT₂GC₂TT₂-3′ |
| | SEQ ID NO: 156 | |
| 1559 | SEQ ID NO: 143 & | 3′-GACGAAGTACG₁G₁G₁A₁A₁G₁A₁T₁G₁-5′-5′-CT₂GC₂T-3′ |
| | SEQ ID NO: 157 | |
| 1560 | SEQ ID NO: 144 & | 3′-GACGAAGTACGG₁G₁A₁A₁G₁A₁T₁G₁-5′-5′-CT₂GC₂TT₂-3′ |
| | SEQ ID NO: 156 | |
| 1561 | SEQ ID NO: 145 & | 3′-GACGAAGTACGG₁G₁A₁A₁G₁A₁T₁G₁-5′-5′-CT₂GC₂T-3′ |
| | SEQ ID NO: 157 | |
| 1562 | SEQ ID NO: 138 & | 3′-GACGAAGTAGG₁G₁G₁A₁A₁G₁A₁T₁G₁-5′-5′-C₂TG₂CT₂T-3′ |
| | SEQ ID NO: 213 | |
| 1563 | SEQ ID NO: 139 & | 3′-GACGAAGTAGG₁G₁G₁A₁A₁G₁A₁T₁G₁-5′-5′-C₂TG₂CT₂-3′ |
| | SEQ ID NO: 214 | |
| 1564 | SEQ ID NO: 140 & | 3′-GACGAAGTAGGGG₁A₁A₁G₁A₁T₁G₁-5′-5′-C₂TG₂CT₂T-3′ |
| | SEQ ID NO: 213 | |
| 1565 | SEQ ID NO: 141 & | 3′-GACGAAGTAGGGG₁A₁A₁G₁A₁T₁G₁-5′-5′-C₂TG₂CT₂-3′ |
| | SEQ ID NO: 214 | |
| 1566 | SEQ ID NO: 142 & | 3′-GACGAAGTACG₁G₁G₁A₁A₁G₁A₁T₁G₁-5′-5′-C₂TG₂CT₂T-3′ |
| | SEQ ID NO: 213 | |
| 1567 | SEQ ID NO: 143 & | 3′-GACGAAGTACG₁G₁G₁A₁A₁G₁A₁T₁G₁-5′-5′-C₂TG₂CT₂-3′ |
| | SEQ ID NO: 214 | |
| 1568 | SEQ ID NO: 144 & | 3′-GACGAAGTACGG₁G₁A₁A₁G₁A₁T₁G₁-5′-5′-C₂TG₂CT₂T-3′ |
| | SEQ ID NO: 213 | |
| 1569 | SEQ ID NO: 145 & | 3′-GACGAAGTACGG₁G₁A₁A₁G₁A₁T₁G₁-5′-5′-C₂TG₂CT₂-3′ |
| | SEQ ID NO: 214 | |
| 1570 | SEQ ID NO: 138 & | 3′-GACGAAGTAGG₁G₁G₁A₁A₁G₁A₁T₁G₁-5′-5′-C₃T₃G₃C₃T₃T₃-3′ |
| | SEQ ID NO: 215 | |
| 1571 | SEQ ID NO: 139 & | 3′-GACGAAGTAGG₁G₁G₁A₁A₁G₁A₁T₁G₁-5′-5′-C₃T₃G₃C₃T₃-3′ |
| | SEQ ID NO: 216 | |
| 1572 | SEQ ID NO: 140 & | 3′-GACGAAGTAGGGG₁A₁A₁G₁A₁T₁G₁-5′-5′-C₃T₃G₃C₃T₃T₃-3′ |
| | SEQ ID NO: 215 | |
| 1573 | SEQ ID NO: 141 & | 3′-GACGAAGTAGGGG₁A₁A₁G₁A₁T₁G₁-5′-5′-C₃T₃G₃C₃T₃-3′ |
| | SEQ ID NO: 216 | |
| 1574 | SEQ ID NO: 142 & | 3′-GACGAAGTACG₁G₁G₁A₁A₁G₁A₁T₁G₁-5′-5′-C₃T₃G₃C₃T₃T₃-3′ |
| | SEQ ID NO: 215 | |
| 1575 | SEQ ID NO: 143 & | 3′-GACGAAGTACG₁G₁G₁A₁A₁G₁A₁T₁G₁-5′-5′-C₃T₃G₃C₃T₃-3′ |
| | SEQ ID NO: 216 | |
| 1576 | SEQ ID NO: 144 & | 3′-GACGAAGTACGG₁G₁A₁A₁G₁A₁T₁G₁-5′-5′-C₃T₃G₃C₃T₃T₃-3′ |
| | SEQ ID NO: 215 | |
| 1577 | SEQ ID NO: 145 & | 3′-GACGAAGTACGG₁G₁A₁A₁G₁A₁T₁G₁-5′-5′-C₃T₃G₃C₃T₃-3′ |
| | SEQ ID NO: 216 | |

GCAT-DNA phosphorothioate linkage; G₁/C₁/A₁/U₁-2′OME phosphorothioate linkage; A₂/T₂/C₂/G2-DNA phosphodiester linkage; G = 7-deaza-G; A₃/T₃/C₃/G₃-RNA phosphodiester linkage

In certain embodiments, the target nucleic acid (target RNA) is the murine sequence of the target. In certain embodiments, the target nucleic acid (target RNA) is the human sequence of the target.

In certain embodiments, the PCSK9 nucleic acid is the murine sequence set forth in GENBANK Accession No. NM_153565.2 (incorporated herein as SEQ ID NO: 57). In certain embodiments, the PCSK9 nucleic acid is the human sequence set forth in GENBANK Accession No. NM_174936.3 (incorporated herein as SEQ ID NO: 58).

In certain embodiments, the PNPLA3 nucleic acid is the murine sequence set forth in GENBANK Accession No. NM_054088.3 (incorporated herein as SEQ ID NO: 80). In certain embodiments, the PCSK9 nucleic acid is the human sequence set forth in GENBANK Accession No. NM_025225.2 (incorporated herein as SEQ ID NO: 81). In certain embodiments, the PCSK9 nucleic acid is the rhesus monkey sequence set forth in GENBANK Accession No. XM 001109144.3 (incorporated herein as SEQ ID NO: 82). In certain embodiments, the PCSK9 nucleic acid is the cynomolgus monkey sequence set forth in GENBANK Accession No. XM_005567051.2 (incorporated herein as SEQ ID NO: 83).

### siRNA

In embodiments, the oligonucleotide of the functional domain is an siRNA. siRNAs comprise short double-stranded RNA from about 15 to about 50 nucleotides in length, preferably about 18 to about 36 nucleotides in length, that are targeted to the RNA.

In embodiments, the invention provides a cyclic structured oligonucleotide (CSO) comprising a functional domain, a cyclizing domain, and a linker, wherein the functional domain and the cyclizing domain are linked at their 5′ or 3′ ends, wherein the functional domain comprises an oligonucleotide between 15 and 50 nucleotides in length and is complementary to targeted RNA; wherein the cyclizing domain comprises an oligonucleotide between 4 and 100 nucleotides in length and is complementary to a sequence of nucleotides within the functional domain and of polarity opposite to the sequence of nucleotides in the functional domain to which it is complementary; wherein functional domain comprises a siRNA.

In embodiments, the cyclizing domain is the same length as the sequence of the strand of the siRNA of the functional domain to which it is complementary. In embodiments, the cyclizing domain is longer than the sequence of the strand of the siRNA of the functional domain to which it is complementary. In embodiments, the cyclizing domain is shorter than the sequence of the strand of the siRNA of the functional domain to which it is complementary.

In embodiments, the cyclizing domain comprises an oligonucleotide between 4 and 50 nucleotides in length. In embodiments, the cyclizing domain comprises an oligonucleotide between 15and 45 nucleotides in length. In embodiments, the cyclizing domain comprises an oligonucleotide between 15 and 45 nucleotides in length. In embodiments, the oligonucleotide of the cyclizing domain is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In embodiments, the cyclizing domain comprises an oligonucleotide between 4 and 10 nucleotides in length. In embodiments, the cyclizing domain comprises an oligonucleotide between 6 and 10 nucleotides in length. In embodiments, the cyclizing domain comprises an oligonucleotide between 6 and 8 nucleotides in length. In embodiments, the oligonucleotide of the cyclizing domain is 4, 5, 6, 7, 8, 9, or 10 nucleotides in length.

In embodiments, the cyclizing domain is at least 95% complementary to the strand of the siRNA that it is complementary. In embodiments, the cyclizing domain is at least 97% complementary to the strand of the siRNA that it is complementary. In embodiments, the cyclizing domain is at least 98% complementary to the strand of the siRNA that it is complementary. In embodiments, the cyclizing domain is at least 99% complementary to the strand of the siRNA that it is complementary. In embodiments, the cyclizing domain is at least 100% complementary to the strand of the siRNA that it is complementary.

The terms “siRNA” and “short interfering RNA” are interchangeable and refer to single-stranded or double-stranded RNA molecules that are capable of inducing RNA interference. siRNA molecules typically have a duplex region that is between 18 and 36 base pairs in length. The design of such siRNAs is within the skill of ordinary artisans.

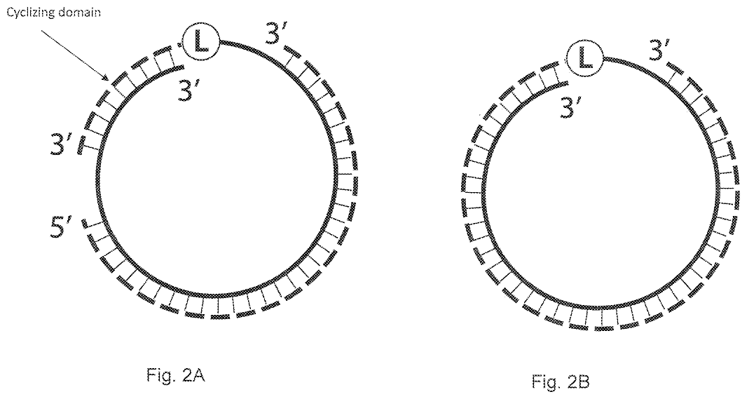

In one embodiment, when the oligonucleotide of the functional domain is an siRNA, the cyclizing domain is attached at the 5′-end of the sense strand of the siRNA through a linker segment (see FIG. 2A). In one embodiment, when the oligonucleotide of the functional domain is an siRNA, the cyclizing domain is attached at the 5′-end of the antisense strand of the siRNA through a linker segment.

In another embodiment, when the oligonucleotide of the functional domain is the antisense strand of the siRNA, the cyclizing domain is the sense strand of the siRNA and is attached at either the 3′- or 5′-end of the antisense strand (see FIGS. 2B and 2C). In other words, in this embodiment, the sense strand of the siRNA also acts as the cyclizing domain.

One or both strands of the siRNA of the invention can also comprise a 3′-overhang. A “3′ overhang” refers to at least one unpaired nucleotide extending from the 3′-end of an RNA strand. Thus, in one aspect, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about two nucleotides in length.

In the case both strands of the siRNA molecule comprise a 3′ overhang, the length of the overhangs can be the same or different for each strand. In a most preferred embodiment, the 3′ overhang is present on both strands of the siRNA and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNAs of the invention can be targeted to any stretch of approximately 18-30, preferably 19-25 contiguous nucleotides of a target mRNA sequence. Techniques for selecting target sequences for siRNA are well known in the art. Thus, the sense strand of the present siRNA comprises a nucleotide sequence identical to any contiguous stretch of about 18 to about 30 nucleotides in the target mRNA.

### Splicing Oligonucleotides

In embodiments, the invention provides a cyclic structured oligonucleotide (CSO) comprising a functional domain, a cyclizing domain, and a linker, wherein the functional domain and the cyclizing domain are linked at their 5′ ends, wherein the functional domain comprises an oligonucleotide between 15 and 45 nucleotides in length and is complementary to targeted RNA; wherein the cyclizing domain comprises an oligonucleotide between 4 and 12 nucleotides in length and is complementary to a sequence of nucleotides within the functional domain and of polarity opposite to the sequence of nucleotides in the functional domain to which it is complementary; wherein functional domain comprises a splicing oligonucleotide and wherein the oligonucleotide of the functional domain is modified.

As used herein, the term “splicing oligonucleotide” refers to an antisense oligonucleotide for modulating splicing. For splice modulation, antisense binds to the target RNA and modulates splicing, thereby the expression of a protein. In the cyclic structured oligonucleotides, the cyclizing domain can be attached either on the 3′ end or the 5′ end to form the cyclic structure. In a preferred embodiment, the cyclizing domain is attached to the 5′ end of the oligonucleotide of the functional domain. The cyclic structure allows reduced protein binding, reduced polyanionic characteristic, and lack of accessibility to the ends which permits endosomal escape and mitigates interaction with pattern recognition receptors.

In embodiments, the oligonucleotide for modulating splicing is a snRNA. The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs. The design of such snRNA is within the skill of ordinary artisans.

In embodiments, the oligonucleotide of the functional domain is as described in WO 2021/055011, which is incorporated herein by reference in its entirety. Specifically, in embodiments, the oligonucleotide of the functional domain comprises an oligonucleotide comprising 14 to 30 linked nucleotides complementary to a target pre-mRNA comprising a retained intron, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof. A CSO having a functional domain comprising an oligonucleotide that modulates splicing is useful for selecting a first mRNA transcript in a gene comprising at least two mRNA transcripts, wherein the oligonucleotide comprises at least 12 contiguous nucleobases complementary to an equal length portion of a target pre-mRNA; wherein the oligonucleotide targets a splice site of the pre-mRNA for a second mRNA transcript thereby blocking the splice site for the second mRNA transcript and directing splicing of the pre-mRNA to the first mRNA transcript. In embodiments, the 2′-substituted nucleotides are selected from 2′ O-methyl ribonucleosides or 2′-methoxyethyl ribonucleosides (MOE).

In embodiments, the splicing oligonucleotide comprises 1 region comprising from 2 to 5 consecutive deoxyribonucleotides, and the remaining nucleotides are 2′-substituted, non-ionic or constrained sugar nucleotides, or combinations thereof. In embodiments, the splicing oligonucleotide comprises 2 regions independently comprising from 2 to 5 consecutive deoxyribonucleotides, and the remaining nucleotides are 2′-substituted, non-ionic, or constrained sugar nucleotides, or combinations thereof. In embodiments, the splicing comprises 3 regions independently comprising from 2 to 5 consecutive deoxyribonucleotides, and the remaining nucleotides are 2′-substituted, non-ionic, or constrained sugar nucleotides, or combinations thereof. In some embodiments, the consecutive deoxyribonucleotides are 2-4 nucleotides in length. In some embodiments, the consecutive deoxyribonucleotides are 4 nucleotides in length.

In embodiments, wherein the consecutive deoxyribonucleotides of the splicing oligonucleotide are at the 5′ end of the antisense oligonucleotide, at the 3′ end of the splicing oligonucleotide, or flanked by at the 2′-substituted, non-ionic, or constrained sugar nucleotides, or combinations thereof. In embodiments, the consecutive deoxyribonucleotides are at the 5′ end of the splicing oligonucleotide. In embodiments, the consecutive deoxyribonucleotides are at the 3′ end of the splicing oligonucleotide. In embodiments, the consecutive deoxyribonucleotides are flanked by the 2′-substituted, non-ionic, or constrained sugar nucleotides, or combinations thereof.

In embodiments, the splicing oligonucleotide of the functional domain comprises 14 to 30 linked nucleotides having at least 12 contiguous nucleobases complementary to an equal length portion of a target pre-mRNA comprising a retained intron, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

In embodiments, the 2′-substituted nucleotides are as described herein. In embodiments, the 2′-substituted nucleotides are selected from 2′ O-methyl ribonucleotides or 2′-MOE.

In embodiments, the oligonucleotide of the functional domain comprises 1 region comprising from 2 to 5 consecutive deoxyribonucleotides. In embodiments, the consecutive deoxyribonucleotides are at the 5′ end of the splicing oligonucleotide, at the 3′ end of the antisense oligonucleotide, flanked by at the 2′-substituted, non-ionic, or constrained sugar nucleotides, or combinations thereof. In embodiments, the consecutive deoxyribonucleotides are at the 5′ end of the splicing oligonucleotide. In embodiments, the consecutive deoxyribonucleotides are at the 3′ end of the splicing oligonucleotide.

In embodiments, the consecutive deoxyribonucleotides are 2-4 nucleotides in length. In embodiments, the consecutive deoxyribonucleotides are 4 nucleotides in length.

In embodiments, an exon flanks the 5′ splice site of the retained intron. In embodiments, an exon flanks the 3′ splice site of the retained intron. In embodiments, an exon flanks the 5′ splice site of the retained intron and an exon flanks the 3′ splice site of the retained intron.

Examples of cyclic oligonucleotides useful for splicing include, but are not limited to, the cyclic oligonucleotides of Table 2. The oligonucleotides in Table 2 are targeted to DMD. Cyclic oligonucleotides with functional domains directed to any other target of interest are well within the skill of one in the art.

TABLE 2

| Compound | | |
|---|---|---|
| # | SEQ ID NO: | Sequence |
| 7 | SEQ ID NO: 158 | 3′-A₂T₂G₂G₂A₂-5′-5′-G₁G₁C₁C₁A₁A₁A₁C₁C₁U₁C₁G₁G₁C₁U₁U₁A₂C₂C₂T₂-3′ |
| | & SEQ ID NO: 7 | |
| 8 | SEQ ID NO: 159 | 3′-A₂A₂T₂G₂G₂A₂-5′-5′-G₁G₁C₁C₁A₁A₁A₁C₁C₁U₁C₁G₁G₁C₁U₁U₁A₂C₂C₂T₂-3′ |
| | & SEQ ID NO: 8 | |
| 9 | SEQ ID NO: 160 | 3′-A₁A₁U₁G₁G₁A₁-5′-5′-G₁G₁C₁C₁A₁A₁A₁C₁C₁U₁C₁G₁G₁C₁U₁U₁A₂C₂C₂T₂-3′ |
| | & SEQ ID NO: 9 | |
| 10 | SEQ ID NO: 10 & | 5′-G₁G₁C₁C₁A₁A₁A₁C₁C₁U₁C₁G₁G₁C₁U₁U₁A₂C₂C₂T₂-3′-3′-C₂C₂G₂G₂T₂-5′ |
| | SEQ ID NO: 161 | |
| 11 | SEQ ID NO: 11 & | 5′-G₁G₁C₁C₁A₁A₁A₁C₁C₁U₁C₁G₁G₁C₁U₁U₁A₂C₂C₂T₂-3′-3′-C₂C₂G₂G₂T₂T₂-5′ |
| | SEQ ID NO: 162 | |
| 12 | SEQ ID NO: 12 & | 5′-G₁G₁C₁C₁A₁A₁A₁C₁C₁U₁C₁G₁G₁C₁U₁U₁A₂C₂C₂T₂-3′-3′-C₁C₁G₁G₁U₁U₁-5′ |
| | SEQ ID NO: 163 | |
| 13 | SEQ ID NO: 158 | 3′-A₂T₂G₂G₂A₂-5-5′-G₂G₂C₂C₁A₁A₁A₁C₁C₁U₁C₁G₁G₁C₁U₁U₁A₁C₁C₁U₁-3′ |
| | & SEQ ID NO: 13 | |
| 14 | SEQ ID NO: 158 | 3′-A₂T₂G₂G₂A₂-5-5′-G₁G₁C₁C₁A₂A₂A₂C₁C₁U₁C₁G₁G₁C₁U₁U₁A₁C₁C₁U₁-3′ |
| | & SEQ ID NO: 14 | |
| 15 | SEQ ID NO: 158 | 3′-A₂T₂G₂G₂A₂-5-5′-G₁G₁C₁C₁A₁A₁A₁C₁C₂T₂C₂G₁G₁C₁U₁U₁A₁C₁C₁U₁-3′ |
| | & SEQ ID NO: 15 | |
| 16 | SEQ ID NO: 158 | 3′-A₂T₂G₂G₂A₂-5-5′-G₁G₁C₁C₁A₁A₁A₁C₁C₁U₁C₁G₁G₂C₂T₂U₁A₁C₁C₁U₁-3′ |
| | & SEQ ID NO: 16 | |
| 17 | SEQ ID NO: 158 | 3′-A₂T₂G₂G₂A₂-5-5′-G₂G₂C₂C₁A₁A₁A₁C₁C₁U₁C₁G₁G₁C₁U₁U₁A₂C₂C₁U₁-3′ |
| | & SEQ ID NO: 17 | |
| 18 | SEQ ID NO: 158 | 3′-A₂T₂G₂G₂A₂-5-5′-G₁G₁C₁C₁A₁A₁A₁C₁C₁U₁C₁G₁G₁C₁U₁U₁A₁C₂C₂T₂-3′ |
| | & SEQ ID NO: 18 | |
| 19 | SEQ ID NO: 158 | 3′-A₂T₂G₂G₂A₂-5-5′-G₂G₂C₂C₂A₁A₁A₁C₁C₁U₁C₁G₁G₁C₁U₁U₁A₁C₁C₁U₁-3′ |
| | & SEQ ID NO: 19 | |
| 20 | SEQ ID NO: 158 | 3′-A₂T₂G₂G₂A₂-5-5′-G₁G₁C₁C₁A₂A₂A₂C₂C₁U₁C₁G₁G₁C₁U₁U₁A₁C₁C₁U₁-3′ |
| | & SEQ ID NO: 20 | |
| 21 | SEQ ID NO: 158 | 3′-A₂T₂G₂G₂A₂-5-5′-G₁G₁C₁C₁A₁A₁A₁C₁C₂T₂C₂G₂G₁C₁U₁U₁A₁C₁C₁U₁-3′ |
| | & SEQ ID NO: 21 | |
| 22 | SEQ ID NO: 158 | 3′-A₂T₂G₂G₂A₂-5-5′-G₁G₁C₁C₁A₁A₁A₁C₁C₁U₁C₁G₁G₂C₂T₂T₂A₁C₁C₁U₁-3′ |
| | & SEQ ID NO: 22 | |
| 23 | SEQ ID NO: 158 | 3′-A₂T₂G₂G₂A₂-5-5′-G₁G₁C₁C₁A₁A₁A₁C₁C₁U₁C₁G₁G₁C₂T₂T₂A₂C₂C₂T₂-3′ |
| | & SEQ ID NO: 23 | |
| 24 | SEQ ID NO: 158 | 3′-A₂T₂G₂G₂A₂-5-5′-G₁G₁C₁C₁A₁A₁A₁C₁C₁U₁C₁G₁G₁C₁T₂T₂A₂C₂C₂T₂-3′ |
| | & SEQ ID NO: 24 | |
| 25 | SEQ ID NO: 158 | 3′-A₂T₂G₂G₂A₂-5-5′-G₁G₁C₁C₁A₁A₁A₁C₁C₁U₁C₁G₁G₁C₁U₁T₂A₂C₂C₂T₂-3′ |
| | & SEQ ID NO: 25 | |
| 26 | SEQ ID NO: 158 | 3′-A₂T₂G₂G₂A₂-5-5′-G₁G₁C₁C₁A₁A₁A₁C₁C₁U₁C₁G₁G₁C₁U₁U₁A₁C₂C₂T₂-3′ |
| | & SEQ ID NO: 26 | |
| 27 | SEQ ID NO: 158 | 3′-A₂T₂G₂G₂A₂-5-5′-G₁G₁C₁C₁A₁A₁A₁C₁C₁U₁C₁G₁G₁C₁U₁U₁A₁C₁C₂T₂-3′ |
| | & SEQ ID NO: 27 | |
| 28 | SEQ ID NO: 28 & | 5′-G₂G₂C₂C₁A₁A₁A₁C₁C₁U₁C₁G₁G₁C₁U₁U₁A₁C₁C₁U₁-3′-3′-C₂C₂G₂G₂T₂-5′ |
| | SEQ ID NO: 161 | |
| 29 | SEQ ID NO: 29 & | 5′-G₁G₁C₁C₁A₂A₂A₂C₁C₁U₁C₁G₁G₁C₁U₁U₁A₁C₁C₁U₁-3′-3′-C₂C₂G₂G₂T₂-5′ |
| | SEQ ID NO: 161 | |
| 30 | SEQ ID NO: 30 & | 5′-G₁G₁C₁C₁A₁A₁A₁C₁C₂T₂C₂G₁G₁C₁U₁U₁A₁C₁C₁U₁-3′-3′-C₂C₂G₂G₂T₂-5′ |
| | SEQ ID NO: 161 | |
| 31 | SEQ ID NO: 31 & | 5′-G₁G₁C₁C₁A₁A₁A₁C₁C₁U₁C₁G₁G₂C₂T₂U₁A₁C₁C₁U₁-3′-3′-C₂C₂G₂G₂T₂-5′ |
| | SEQ ID NO: 161 | |
| 32 | SEQ ID NO: 32 & | 5′-G₂G₂C₂C₁A₁A₁A₁C₁C₁U₁C₁G₁G₁C₁U₁U₁A₂C₂C₁U₁-3′-3′-C₂C₂G₂G₂T₂-5′ |
| | SEQ ID NO: 161 | |
| 33 | SEQ ID NO: 33 & | 5′-G₁G₁C₁C₁A₁A₁A₁C₁C₁U₁C₁G₁G₁C₁U₁U₁A₁C₂C₂T₂-3′-3′-C₂C₂G₂G₂T₂-5′ |
| | SEQ ID NO: 161 | |
| 34 | SEQ ID NO: 34 & | 5′-G₂G₂C₂C₂A₁A₁A₁C₁C₁U₁C₁G₁G₁C₁U₁U₁A₁C₁C₁U₁-3′-3′-C₂C₂G₂G₂T₂-5′ |
| | SEQ ID NO: 161 | |
| 35 | SEQ ID NO: 35 & | 5′-G₁G₁C₁C₁A₂A₂A₂C₂C₁U₁C₁G₁G₁C₁U₁U₁A₁C₁C₁U₁-3′-3′-C₂C₂G₂G₂T₂-5′ |
| | SEQ ID NO: 161 | |
| 36 | SEQ ID NO: 36 & | 5′-G₁G₁C₁C₁A₁A₁A₁C₁C₂T₂C₂G₂G₁C₁U₁U₁A₁C₁C₁U₁-3′-3′-C₂C₂G₂G₂T₂-5′ |
| | SEQ ID NO: 161 | |
| 37 | SEQ ID NO: 37 & | 5′-G₁G₁C₁C₁A₁A₁A₁C₁C₁U₁C₁G₁G₂C₂T₂T₂A₁C₁C₁U₁-3′-3′-C₂C₂G₂G₂T₂-5′ |
| | SEQ ID NO: 161 | |
| 38 | SEQ ID NO: 38 & | 5′-G₁G₁C₁C₁A₁A₁A₁C₁₁CU₁C₁G₁G₁C₂T₂T₂A₂C₂C₂T₂-3′-3′-C₂C₂G₂G₂T₂-5′ |
| | SEQ ID NO: 161 | |
| 39 | SEQ ID NO: 39 & | 5′-G₁G₁C₁C₁A₁A₁A₁C₁C₁U₁C₁G₁G₁C₁T₂T₂A₂C₂C₂T₂-3′-3′-C₂C₂G₂G₂T₂-5′ |
| | SEQ ID NO: 161 | |
| 40 | SEQ ID NO: 40 & | 5′-G₁G₁C₁C₁A₁A₁A₁C₁C₁U₁C₁G₁G₁C₁U₁T₂A₂C₂C₂T₂-3′-3′-C₂C₂G₂G₂T₂-5′ |
| | SEQ ID NO: 161 | |
| 41 | SEQ ID NO: 41 & | 5′-G₁G₁C₁C₁A₁A₁A₁C₁C₁U₁C₁G₁G₁C₁U₁U₁A₁C₂C₂T₂-3′-3′-C₂C₂G₂G₂T₂-5′ |
| | SEQ ID NO: 161 | |
| 42 | SEQ ID NO: 42 & | 5′-G₁G₁C₁C₁A₁A₁A₁C₁C₁U₁C₁G₁G₁C₁U₁U₁A₁C₁C₂T₂-3′-3′-C₂C₂G₂G₂T₂-5′ |
| | SEQ ID NO: 161 | |
| 43 | SEQ ID NO: 164 | 3′-A₁U₁G₁G₁A₁-5′-5′-G₁G₁C₁C₁A₁A₁A₁C₁C₁U₁C₁G₁G₁C₁U₁U₁A₂C₂C₂T₂-3′ |
| | & SEQ ID NO: 43 | |
| 44 | SEQ ID NO: 44 & | 5′-G₁G₁C₁C₁A₁A₁A₁C₁C₁U₁C₁G₁G₁C₁U₁U₁A₂C₂C₂T₂-3′-3′-C₁C₁G₁G₁U₁-5′ |
| | SEQ ID NO: 165 | |

G₁/C₁/A₁/U₁-2′OME nucleotide; A₂/T₂/C₂/G₂-deoxyribonucleotide

In embodiments, the invention provides a cyclic structured oligonucleotide (CSO) comprising a functional domain, a cyclizing domain, and a linker, wherein the functional domain and the cyclizing domain are linked at their 5′ ends, wherein the functional domain comprises an oligonucleotide between 15 and 500 nucleotides in length and is complementary to targeted RNA; wherein the cyclizing domain comprises an oligonucleotide between 4 and 12 nucleotides in length and is complementary to a sequence of nucleotides within the functional domain and of polarity opposite to the sequence of nucleotides in the functional domain to which it is complementary; wherein functional domain comprises an antisense oligonucleotide of an adenosine deaminase acting on RNA (ADAR) system.

In some embodiments, the oligonucleotide of the functional domain is an antisense oligonucleotide that recruit endogenous ADAR (adenosine deaminase acting on RNA) enzymes to edit endogenous transcripts. ADARs are a group of enzymes that catalyze the conversion of adenosines (A's) to inosines (I's) in a process known as RNA editing. Though ADARs can act on different types of RNA, editing events in coding regions of mRNA are of particular interest as I's base pair like guanosines (G's). Thus, every A-to-I change catalyzed by ADAR is read as an A-to-G change during translation, potentially altering protein sequence and function. This ability to re-code makes ADAR an attractive therapeutic tool to correct genetic mutations within mRNA.

The oligonucleotide of an ADAR system itself comprises one or more domains. One domain, known as the complementary domain, comprises a region of consecutive nucleotides that are complementary to the target RNA. In embodiments, the complementary domain is from about 15 to about 120 nucleotides in length. In embodiments, the complementary domain is from about 17 to about 60 nucleotides in length. Another domain, known as the recruiting domain, comprises a region of the oligonucleotide that recruits ADAR enzymes.

### CRISPR

In embodiments, the invention provides a cyclic structured oligonucleotide (CSO) comprising a functional domain, a cyclizing domain, and a linker, wherein the functional domain and the cyclizing domain are linked at their 5′ ends, wherein the functional domain comprises an oligonucleotide between 17 and 500 nucleotides in length and is complementary to targeted RNA; wherein the cyclizing domain comprises an oligonucleotide between 4 and 12 nucleotides in length and is complementary to a sequence of nucleotides within the functional domain and of polarity opposite to the sequence of nucleotides in the functional domain to which it is complementary; wherein functional domain comprises an antisense oligonucleotide of a CRISPR-based system.

The oligonucleotide of the functional domain is an antisense oligonucleotide that functions as a guideRNA for a CRISPR-based system. The terms “sgRNA” and “guideRNA” are interchangeable and refer to a specific RNA sequence that recognizes the target DNA or RNA region of interest and directs the endonuclease there for editing. The gRNA is usually made up of two parts: crispr RNA (crRNA), a 17-30 nucleotide sequence complementary to the target DNA, and a tracr RNA, which serves as a binding scaffold for the Cas nuclease.

Any suitable engineered sgRNA, or crRNA and tracrRNA, can be employed as long as it is effective for recognizing a target DNA or RNA. The design of such sgRNA, or crRNA and tracrRNA is within the skill of ordinary artisans.

### Immunostimulatory Oligonucleotides

In embodiments, the invention provides a cyclic structured oligonucleotide (CSO) comprising a functional domain, a cyclizing domain, and a linker, wherein the functional domain and the cyclizing domain are linked at their 3′ ends, wherein the functional domain comprises an oligonucleotide between 11 and 400 nucleotides in length; wherein the cyclizing domain comprises an oligonucleotide between 4 and 400 nucleotides in length and is complementary to a sequence of nucleotides within the functional domain and of polarity opposite to the sequence of nucleotides in the functional domain to which it is complementary; wherein functional domain comprises an immunostimulatory oligonucleotide.

In embodiments, the immunostimulatory oligonucleotide is capable of inducing an interferon response in a vertebrate cell.

In embodiments, the nucleotide sequence of the immunostimulatory oligonucleotide is not complementary to and does not bind to another nucleotide sequence, for example, a target RNA. In this embodiment, the nucleotide sequence of the immunostimulatory oligonucleotide is not, for example, an antisense oligonucleotide and does not have antisense activity.

In embodiments, the immunostimulatory oligonucleotide is between 11 and 40 nucleotides in length. In embodiments, immunostimulatory oligonucleotide is between 15 and 28 nucleotides in length. In embodiments, immunostimulatory oligonucleotide is between 17 and 25 nucleotides in length.

In embodiments, the cyclizing domain is the same length as the sequence of the functional domain to which it is complementary. In embodiments, the cyclizing domain is longer than the sequence of the functional domain to which it is complementary. In embodiments, the cyclizing domain is shorter than the sequence of the functional domain to which it is complementary.

In embodiments, the cyclizing domain is between 11 and 40 nucleotides in length. In embodiments, the cyclizing domain is between 15 and 28 nucleotides in length. In embodiments, the cyclizing domain is between 17 and 25 nucleotides in length. In embodiments, the cyclizing domain is between 4 and 12 nucleotides in length. In embodiments, the cyclizing domain is between 4 and 10 nucleotides in length. In embodiments, the cyclizing domain is between 4 and 8 nucleotides in length.

In embodiments, the internucleotide linkages of the immunostimulatory oligonucleotide are phosphorothioate, phosphodiester, or combinations thereof.

In embodiments, the internucleotide linkages of the immunostimulatory oligonucleotide are phosphorothioate internucleotide linkages.

In embodiments, the internucleotide linkages of the immunostimulatory oligonucleotide are phosphodiester.

In embodiments, the internucleotide linkages of the immunostimulatory oligonucleotide are a combination of phosphorothioate and phosphodiester internucleotide linkages.

In embodiments, the internucleotide linkages of the cyclizing domain are phosphorothioate, phosphodiester, or combinations thereof.

In embodiments, the internucleotide linkages of the cyclizing domain are phosphorothioate internucleotide linkages.

In embodiments, the internucleotide linkages of the cyclizing domain are phosphodiester.

In embodiments, the internucleotide linkages of the cyclizing domain are a combination of phosphorothioate and phosphodiester internucleotide linkages.

In embodiments, the internucleotide linkages of the immunostimulatory oligonucleotide are phosphorothioate and the internucleotide linkages of the cyclizing domain are phosphodiester, or vice versa.

Immunostimulatory oligonucleotides include, but are not limited to, oligonucleotides that induce immunostimulation through endosomal toll-like receptors, RIG like receptors, STING, cGAS and inflammasomes. Toll-like receptors (TLRs) are pattern recognition receptors (PRRs) which play a crucial in the initiation of innate immune response by detecting potential harmful pathogens. Each TLR has a broad range of specificities, for example, TLR1, 2, 4 and 6 recognize bacterial lipids, TLR3, 7 and 8 recognize viral RNA, TLR9 recognizes bacterial DNA comprising a CG motif, and TLR5 and 10 recognize bacterial or parasite proteins. The design of such immunostimulatory oligonucleotides is within the skill of ordinary artisans.

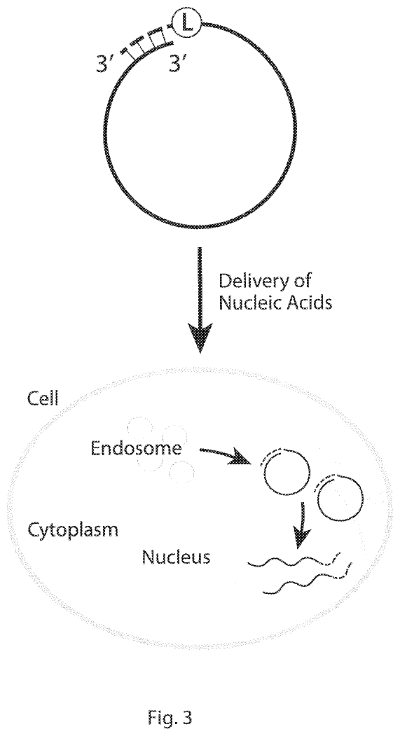

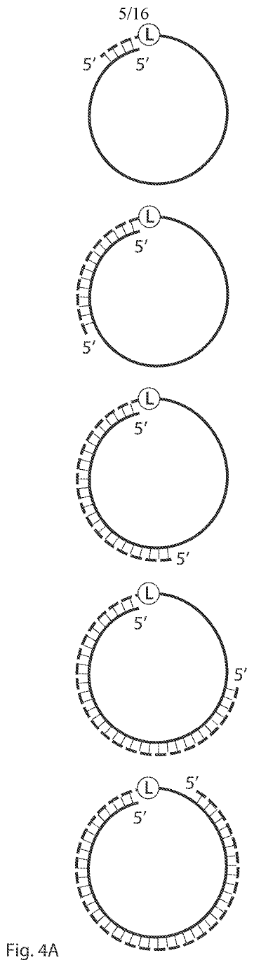

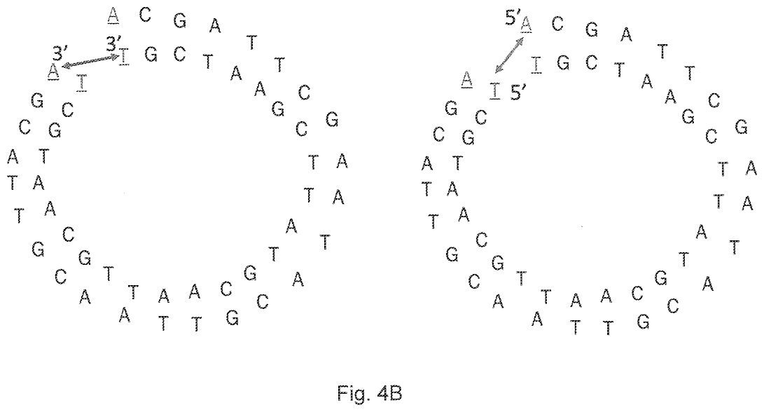

In embodiments, when the oligonucleotide of the functional domain is an immunostimulatory oligonucleotide, the cyclizing domain is attached at the 5′-end or the 3′ end of the functional domain through a linker segment (see e.g., FIG. 3). In embodiments, the cyclizing domain is attached at the 5′ end. In embodiments, the cyclizing domain is attached at the 3′ end.

In another embodiment, the oligonucleotide of the functional domain is an immunostimulatory oligonucleotide, the cyclizing domain is another immunostimulatory oligonucleotide wherein the immunostimulatory oligonucleotide of the cyclizing domain is complementary to a sequence of nucleotides within the functional domain and of polarity opposite to the sequence of nucleotides in the functional domain to which it is complementary and is attached at the 3′end of the functional domain. In other words, in this embodiment, the cyclizing domain also acts as an immunostimulatory oligonucleotide.

Examples of cyclic oligonucleotides useful for immunostimulation include, but are not limited to, the cyclic oligonucleotides of Table 3. The oligonucleotides in Table 3 comprise a CpG dinucleotide and activate Toll-like receptor 9 (TLR-9). Cyclic oligonucleotides with functional domains comprising an immunostimulatory oligonucleotide directed to any other PRR or TLR of interest are well within the skill of one in the art.

TABLE 3

| Compound | | |
|---|---|---|
| # | SEQ ID NO: | Sequence |
| 45 | SEQ ID NO: 166 | 3′-TAGCA-5′-5′-TCGTAACGTTACGTATTCGAATCGT-3′ |
| | & SEQ ID NO: 45 | |
| 46 | SEQ ID NO: 167 | 3′-TTAGCA-5′-5′-TCGTAACGTTACGTATTCGAATCGT-3′′ |
| | & SEQ ID NO: 46 | |
| 47 | SEQ ID NO: 168 | 3′-ATGCT-5′-5′-ACGATTCGAATACGTAACGTTACGA-3′ |
| | & SEQ ID NO: 47 | |
| 48 | SEQ ID NO: 169 | 3′-AATGCT-5′-5′-ACGATTCGAATACGTAACGTTACGA-3′ |
| | & SEQ ID NO: 48 | |
| 49 | SEQ ID NO: 170 | 5′-TTACGA-3′-3′-TGCTAAGCTTATGCATTGCAATGCT-5′ |
| | & SEQ ID NO: 49 | |
| 50 | SEQ ID NO: 171 | 5′-TACGA-3′-3′-TGCTAAGCTTATGCATTGCAATGCT-5′ |
| | & SEQ ID NO: 50 | |
| 51 | SEQ ID NO: 172 | 5′-AATGCT-3′-3′-AGCATTGCAATGCATAAGCTTAGCA-5′ |
| | & SEQ ID NO: 51 | |
| 52 | SEQ ID NO: 173 | 5′-ATGCT-3′-3′-AGCATTGCAATGCATAAGCTTAGCA-5′ |
| | & SEQ ID NO: 52 | |
| 53 | SEQ ID NO: 53 & | 5′-TCGTAACGTTACGTATTCGAATCGT-3′-3′- |
| | SEQ ID NO: 55 | AGCATTGCAATGCATAAGCTTAGCA-5′ |
| 54 | SEQ ID NO: 54 & | 3′-AGCATTGCAATGCATAAGCTTAGCA-5′-5′- |
| | SEQ ID NO: 56 | TCGTAACGTTACGTATTCGAATCGT-3′ |
| 55 | SEQ ID NO: 84 & | 5′-T₂C₂G₂T₂A₂A₂C₂G₂T₂T₂A₂C₂G₂T₂A₂T₂T₂C₂G₂A₂A₂T₂C₂G₂T₂-3′-3′- |
| | SEQ ID NO: 85 | A₂G₂C₂A₂T₂T₂G₂C₂A₂A₂T₂G₂C₂A₂T₂A₂A₂G₂C₂T₂T₂A₂G₂C₂A₂-5′ |
| 1101 | SEQ ID NO: 86 & | 5′-TCGTATCGTAACGTATTCGAATCGT-3′-3′- |
| | SEQ ID NO: 174 | AGCATAGCATTGCATAAGCTTAGCA-5′ |
| 1102 | SEQ ID NO: 87 & | 5′-T₂C₂G₂T₂A₂T₂CGTAACGTATTCGAATCGT-3′-3′- |
| | SEQ ID NO: 175 | AGCATAGCATTGCATAAGCT₂T₂A₂G₂C₂A₂-5′ |
| 1103 | SEQ ID NO: 88 & | 5′-TCGACTTGAACGATTCGAATACGTAAC-3′-3′- |
| | SEQ ID NO: 176 | AGCTGAACTTGCTAAGCTTATGCATTG-5′ |
| 1104 | SEQ ID NO: 89 & | 5′-TCGACUTGAACGATTCGAATACGTAAC-3′-3′- |
| | SEQ ID NO: 177 | AGCTGAACTTGCTAAGCTTATGCATTG-5′ |
| 1105 | SEQ ID NO: 90 & | 5′-TCGACTTIAACGATTCGAATACGTAAC-3′-3′- |
| | SEQ ID NO: 178 | AGCTGAACTTGCTAAGCTTATGCATTG-5′ |
| 1106 | SEQ ID NO: 91 & | 5′-T₂C₂G₂A₂C₂T₂TGAACGATTCGAATACGTAAC-3′-3′- |
| | SEQ ID NO: 179 | AGCTGAACTTGCTAAGCTTATG₂C₂A₂T₂T₂G₂-5′ |
| 1107 | SEQ ID NO: 92 & | 5′-TCGACTTGAACGATTCGAATACGTAAC-3′-3′- |
| | SEQ ID NO: 180 | AGCTGAACTTGCTAAGCTTAT-5′ |
| 1108 | SEQ ID NO: 93 & | 5′-TCGACTTGAACGATTCGAATACGTAAC-3′-3′- |
| | SEQ ID NO: 181 | AGCTGAACTTGCTAAGCT-5′ |
| 1109 | SEQ ID NO: 94 & | 5′-TCGACTTGAACGATTCGAATACGTAAC-3′-3′- |
| | SEQ ID NO: 182 | AGCTGAACTTGCT-5′ |
| 1110 | SEQ ID NO: 95 & | 5′-TCGACTTGAACGATTCGAATACGTAAC-3′-3′-AGCTGA-5′ |
| | SEQ ID NO: 183 | |
| 1111 | SEQ ID NO: 96 & | 5′-T₂C₂G₂A₂C₂T₂TGAACGATTCGAATACGTAAC-3′-3′- |
| | SEQ ID NO: 184 | A₂G₂C₂T₂G₂A₂-5′ |
| 1112 | SEQ ID NO: 97 | 5′-TCGACTTGAACGATTCGAATACGTAAC-3′ |
| 1113 | SEQ ID NO: 98 & | 5′-TCGACTTGAACGATTCGAATACGTAAC-3′-L-3′- |
| | SEQ ID NO: 185 | AGCTGAACTTGCTAAGCTTATGCATTG-5′ |
| 1114 | SEQ ID NO: 99 & | 5′-T₂C₂G₂A₂C₂T₂T₂G₂A₂A₂C₂G₂A₂T₂T₂C₂G₂A₂A₂T₂A₂C₂G₂T₂A₂A₂C₂-3′- |
| | SEQ ID NO: 186 | L-3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| 1115 | SEQ ID NO: 100 & | 5′-TCGACTTGA₂A₂C₂G₂A₂T₂T₂C₂G₂A₂A₂T₂A₂C₂G₂T₂A₂A₂C₂-3′-L-3′- |
| | SEQ ID NO: 187 | A₂G₂C₂T₂G₂A₂A₂C₂T₂T₂G₂C₂T₂A₂A₂G₂C₂T₂T₂ATGCATTG-5′ |
| 1116 | SEQ ID NO: 101 & | 5′-TCGACTTGAACGATTCGAATACGTAAC-3′-L-3′- |
| | SEQ ID NO: 188 | AGCTGAACTTGCTAAGCTTAT-5′ |
| 1117 | SEQ ID NO: 102 & | 5′-TCG ACTTGAACGATTCGAATACGTAAC-3′-L-3′- |
| | SEQ ID NO: 189 | AGCTGAACTTGCTAAGCT-5′ |
| 1118 | SEQ ID NO: 103 & | 5′-TCGACTTGAACGATTCGAATACGT AAC-3′-L-3′- |
| | SEQ ID NO: 190 | AGCTGAACTTGCT-5′ |
| 1119 | SEQ ID NO: 104 & | 5′-TCGACTTGAACGATTCGAATACGT AAC-3′-L-3′- |
| | SEQ ID NO: 191 | A₂G₂C₂T₂G₂A₂A₂C₂T₂T₂G₂C₂T₂-5′ |
| 1120 | SEQ ID NO: 105 & | 5′-TCG ACTTGAACGATTCGAATACGTAAC-3′L-3′-AGCTGA-5′ |
| | SEQ ID NO: 192 | |
| 1121 | SEQ ID NO: 106 | 3′-AGCTGAACTTGCTAAGCTTATGCATTG-5′ |
| 1122 | SEQ ID NO: 107 & | 3′-AGCTGAACTTGCTAAGCTTATGCATTG-5′-5′- |
| | SEQ ID NO: 193 | TCGACTTGAACGATTCGAATACGTAAC-3′ |
| 1123 | SEQ ID NO: 108 & | 3′-AGCTGAACTTGCTAAGCTTATGCATTG-5′-5′-TCGACT-3′ |
| | SEQ ID NO: 194 | |
| 1124 | SEQ ID NO: 109 & | 5′-T₂C₂G₂T₂A₂T₂C₂G₂T₂A₂A₂C₂G₂T₂A₂T₂T₂C₂G₂A₂A₂T₂C₂G₂T₂-3′-3′- |
| | SEQ ID NO: 195 | A₂G₂C₂A₂T₂A₂G₂C₂A₂T₂T₂G₂C₂A₂T₂A₂A₂G₂C₂T₂T₂A₂G₂C₂A₂-5′ |
| 1125 | SEQ ID NO: 110 | 5′-T₂C₂G₂A₂C₂T₂T₂G₂A₂A₂C₂G₂A₂T₂T₂C₂G₂A₂A₂T₂A₂C₂G₂T₂A₂A₂C₂-3′- |
| | & SEQ ID NO: 196 | 3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| 1126 | SEQ ID NO: 111 & | 5′-T₂C₂G₂A₂C₂T₂T₂G₂A₂A₂C₂G₂A₂T₂T₂C₂G₂A₂A₂T₂A₂C₂G₂T₂A₂A₂C₂- |
| | SEQ ID NO: 197 | 3′-3′-A₂G₂C₂T₂G₂A₂A₂C₂T₂T₂G₂C₂T₂A₂A₂G₂C₂T₂T₂A₂T₂-5′ |
| 1127 | SEQ ID NO: 112 & | 3′-A₂G₂C₂T₂G₂A₂A₂C₂T₂T₂G₂C₂T₂A₂A₂G₂C₂T₂T₂A₂T₂G₂C₂A₂T₂T₂G₂-5′- |
| | SEQ ID NO: 198 | 5′-T₂C₂G₂A₂C₂T₂T₂G₂A₂A₂C₂G₂A₂T₂T₂C₂G₂A₂A₂T₂A₂C₂G₂T₂A₂A₂C₂-3′ |
| 1128 | SEQ ID NO: 113 & | 5′-T₂C₂G₂A₂C₂T₂T₂G₂A₂A₂C₂G₂A₂T₂T₂C₂G₂A₂A₂T₂A₂C₂G₂T₂A₂A₂C₂-3′- |
| | SEQ ID NO: 199 | 3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| 1129 | SEQ ID NO: 114 & | 5′-T₂C₂G₂A₂C₂T₂T₂G₂A₂A₂C₂G₂A₂T₂T₂C₂G₂A₂A₂T₂A₂C₂G₂T₂A₂A₂C₂- |
| | SEQ ID NO: 200 | 3′-3′-A₂G₂C₂T₂G₂A₂A₂C₂T₂T₂G₂C₂T₂A₂A₂G₂C₂T₂T₂A₂T₂G₂C₂A₂T₂-5′ |
| 1130 | SEQ ID NO: 115 & | 5′-T₂C₂G₂A₂C₂T₂T₂G₂A₂A₂C₂G₂A₂T₂T₂C₂G₂A₂A₂T₂A₂C₂G₂T₂A₂A₂C₂- |
| | SEQ ID NO: 201 | 3′-3′-A₂G₂C₂T₂G₂A₂A₂C₂T₂T₂G₂C₂T₂A₂A₂G₂C₂T₂T₂A₂T₂G₂C₂A₂-5′ |
| 1131 | SEQ ID NO: 116 | 5′-T₂C₂G₂A₂C₂T₂T₂G₂A₂A₂C₂G₂A₂T₂T₂C₂G₂A₂A₂T₂A₂C₂G₂T₂A₂A₂C₂- |
| | | 3′ |
| 1132 | SEQ ID NO: 117 | 3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| 1133 | SEQ ID NO: 118 & | 5′-T₂C₂G₂A₂C₂T₂T₂G₂A₂A₂C₂G₂A₂T₂T₂C₂G₂A₂A₂T₂A₂C₂G₂T₂A₂A₂C₂-3′- |
| | SEQ ID NO: 202 | 3′-A₂C₂G₂T₂G₂A₂A₂C₂T₂T₂C₂G₂T₂A₂A₂C₂G₂T₂T₂A₂T₂C₂G₂A₂T₂T₂G₂-5′ |
| 1134 | SEQ ID NO: 119 & | 5′-TCGACTTGAACGATTCGAATACGTAAC-3′-L-3′- |
| | SEQ ID NO: 203 | A₃G₃C₃T₃G₃A₃A₃C₃T₃T₃G₃C₃T₃A₃A₃G₃C₃T₃T₃A₃T₃G₃C₃A₃T₃T₃G₃-5′ |
| 1135 | SEQ ID NO: 120 & | 5′-TCGACTTGAACGATTCGAATACGTAAC-3′-L-3′- |
| | SEQ ID NO: 204 | A₃G₃C₃T₃G₃A₃A₃C₃T₃T₃G₃C₃T₃A₃A₃G₃C₃T₃-5′ |
| 1136 | SEQ ID NO: 217 & | 5′-T₃C₃G₃A₃C₃T₃T₃G₃A₃A₃C₃G₃A₃T₃T₃C₃G₃A₃A₃T₃A₃C₃G₃T₃A₃A₃C₃- |
| | SEQ ID NO: 204 | 3′-L-3′-A₃G₃C₃T₃G₃A₃A₃C₃T₃T₃G₃C₃T₃A₃A₃G₃C₃T₃-5′ |

GCAT-DNA phosphorothioate linkage; A₂/T₂/C₂/G₂-DNA phosphodiester linkage; A₃/T₃/C₃/G₃-RNA phosphodiester linkage; U = deoxy uridine; I = deoxy inosine; L = triethylene glycol

### Immune Antagonist

In embodiments, the invention provides a cyclic structured oligonucleotide (CSO) comprising a functional domain, a cyclizing domain, and a linker, wherein the functional domain and the cyclizing domain are linked at their 3′ ends, wherein the functional domain comprises an oligonucleotide between 11 and 400 nucleotides in length; wherein the cyclizing domain comprises an oligonucleotide between 4 and 400 nucleotides in length and is complementary to a sequence of nucleotides within the functional domain and of polarity opposite to the sequence of nucleotides in the functional domain to which it is complementary; wherein functional domain comprises an immune antagonist oligonucleotide.

In embodiments, the immune antagonist oligonucleotide is capable of blocking an interferon response in a vertebrate cell.

In embodiments, the nucleotide sequence of the immune antagonist oligonucleotide is not complementary to and does not bind to another nucleotide sequence, for example, a target RNA. In this embodiment, the nucleotide sequence of the immune antagonist oligonucleotide is not, for example, an antisense oligonucleotide and does not have antisense activity.

In embodiments, the immunostimulatory oligonucleotide is between 11 and 30 nucleotides in length. In embodiments, immunostimulatory oligonucleotide is between 15 and 28 nucleotides in length. In embodiments, immunostimulatory oligonucleotide is between 17 and 25 nucleotides in length.

In embodiments, the cyclizing domain is the same length as the sequence of the functional domain to which it is complementary. In embodiments, the cyclizing domain is longer than the sequence of the functional domain to which it is complementary. In embodiments, the cyclizing domain is shorter than the sequence of the functional domain to which it is complementary.

In embodiments, the cyclizing domain is between 11 and 40 nucleotides in length. In embodiments, the cyclizing domain is between 15 and 28 nucleotides in length. In embodiments, the cyclizing domain is between 17 and 25 nucleotides in length. In embodiments, the cyclizing domain is between 4 and 12 nucleotides in length. In embodiments, the cyclizing domain is between 4 and 10 nucleotides in length. In embodiments, the cyclizing domain is between 4 and 8 nucleotides in length.

In embodiments, the internucleotide linkages of the immune antagonist oligonucleotide are phosphorothioate, phosphodiester, or combinations thereof.

In embodiments, the internucleotide linkages of the immune antagonist oligonucleotide are phosphorothioate internucleotide linkages.

In embodiments, the internucleotide linkages of the immune antagonist oligonucleotide are phosphodiester.

In embodiments, the internucleotide linkages of the immune antagonist oligonucleotide are a combination of phosphorothioate and phosphodiester internucleotide linkages.

In embodiments, the internucleotide linkages of the cyclizing domain are phosphorothioate, phosphodiester, or combinations thereof.

In embodiments, the internucleotide linkages of the cyclizing domain are phosphorothioate internucleotide linkages.

In embodiments, the internucleotide linkages of the cyclizing domain are phosphodiester.

In embodiments, the internucleotide linkages of the cyclizing domain are a combination of phosphorothioate and phosphodiester internucleotide linkages.

In embodiments, the internucleotide linkages of the immune antagonist oligonucleotide are phosphorothioate and the internucleotide linkages of the cyclizing domain are phosphodiester, or vice versa.

Immune antagonist oligonucleotides include, but are not limited to, oligonucleotides that block immunostimulation through endosomal toll-like receptors, RIG like receptors, STING, cGAS and inflammasomes. Toll-like receptors (TLRs) are pattern recognition receptors (PRRs) which play a crucial in the initiation of innate immune response by detecting potential harmful pathogens. Each TLR has a broad range of specificities, for example, TLR1, 2, 4 and 6 recognize bacterial lipids, TLR3, 7 and 8 recognize viral RNA, TLR9 recognizes bacterial DNA comprising a CG motif, and TLR5 and 10 recognize bacterial or parasite proteins. The design of such immune antagonist oligonucleotides is within the skill of ordinary artisans.

In another embodiment, the oligonucleotide of the functional domain is an immune antagonist oligonucleotide, the cyclizing domain is another immune antagonist oligonucleotide wherein the immune antagonist oligonucleotide of the cyclizing domain is complementary to a sequence of nucleotides within the functional domain and of polarity opposite to the sequence of nucleotides in the functional domain to which it is complementary and is attached at the 3′-end of the functional domain (see FIG. 3B). In other words, in this embodiment, the cyclizing domain also acts as an immune antagonist oligonucleotide.

Examples of cyclic oligonucleotides useful for immunostimulation include, but are not limited to, the cyclic oligonucleotides of Table 4. The oligonucleotides in Table 4 comprise a methylated C within the CpG dinucleotide and block activation of Toll-like receptor 9 (TLR-9). Cyclic oligonucleotides with functional domains comprising an immune antagonist oligonucleotide directed to any other PRR or TLR of interest are well within the skill of one in the art.

TABLE 4

| Compound | | |
|---|---|---|
| # | SEQ ID NO: | Sequence |
| 1201 | SEQ ID NO: 121 & | 5′-TCₘGACTTGAACₘGATTCₘGAATACₘGTAAC-3′-3′- |
| | SEQ ID NO: 205 | AGCₘTGAACTTGCₘTAAGCₘTTATGCₘATTG-5′ |
| 1202 | SEQ ID NO: 122 & | 5′-TCₘGACTTGAACₘGATTCₘGAATACₘGTAAC-3′-3′- |
| | SEQ ID NO: 206 | AGCₘTGAACTTGCₘTAAGCₘTTAT-5′ |
| 1203 | SEQ ID NO: 123 & | 5′-TCₘGACTTGAACₘGATTCₘGAATACₘGTAAC-3′-3′- |
| | SEQ ID NO: 207 | AGCₘTGAACTTGCₘTAAGCₘT-5′ |
| 1204 | SEQ ID NO: 124 & | 5′-TCₘGACTTGAACₘGATTCₘGAATACₘGTAAC-3′-3′- |
| | SEQ ID NO: 208 | AGCₘTGAACTTGCₘT-5′ |
| 1205 | SEQ ID NO: 125 & | 5′-TCₘGACTTGAACₘGATTCₘGAATACₘGTAAC-3′-3′- |
| | SEQ ID NO: 209 | AGCₘTGA-5′ |
| 1206 | SEQ ID NO: 126 | 5′-TCₘGACTTGAACₘGATTCₘGAATACₘGTAAC-3′ |
| 1207 | SEQ ID NO: 127 & | 5′-TCₘGACTTGAACₘGATTCₘGAATACₘGTAAC-3′-3′- |
| | SEQ ID NO: 210 | AGCTGAACTTG-5′ |
| 1208 | SEQ ID NO: 128 & | 5′-TCₘGACTTGAACₘGATTCₘGAATACₘGTAAC-3′- |
| | SEQ ID NO: 211 | 3′-AGCTGAAC-5′ |
| 1209 | SEQ ID NO: 129 & | 5′-TCₘGACTTGAACₘGATTCₘGAATACₘGTAAC-3′-3′- |
| | SEQ ID NO: 212 | AGCTGA-5 |

Cₘ = 5′-methylated C

Examples of cyclizing domains useful in the cyclic oligonucleotides described herein include, but are not limited to, the cyclizing domains of Table 5.

TABLE 5

| SEQ ID NO: | Sequence |
|---|---|
| SEQ ID NO: 146 | 3′-T₂C₂G₂T₂G₂G-5′ |
| SEQ ID NO: 147 | 3′-T₂C₂G₂T₂G₂G₂-5′ |
| SEQ ID NO: 148 | 3′-C₂C₂A₂G₂A₂G-5′ |
| SEQ ID NO: 149 | 5′-CTGCTTC-3′ |
| SEQ ID NO: 150 | 5′-CTGCTT-3′ |
| SEQ ID NO: 151 | 5′-CTGCT-3′ |
| SEQ ID NO: 152 | 5′-CTGC-3′ |
| SEQ ID NO: 153 | 5′-C₂T₂G₂C₂T₂T₂-3′ |
| SEQ ID NO: 154 | 5′-C₂T₂G₂C₂T₂-3′ |
| SEQ ID NO: 155 | 5′-C₂T₂G₂C₂-3′ |
| SEQ ID NO: 156 | 5′-CT₂GC₂TT₂-3′ |
| SEQ ID NO: 157 | 5′-CT₂GC₂T-3′ |
| SEQ ID NO: 158 | 3′-A₂T₂G₂G₂A₂-5′ |
| SEQ ID NO: 159 | 3′-A₂A₂T₂G₂G₂A₂-5′ |
| SEQ ID NO: 160 | 3′-A₁A₁U₁G₁G₁A₁-5′ |
| SEQ ID NO: 161 | 3′-C₂C₂G₂G₂T₂-5′ |
| SEQ ID NO: 162 | 3′-C₂C₂G₂G₂T₂T₂-5′ |
| SEQ ID NO: 163 | 3′-C₁C₁G₁G₁U₁U₁-5′ |
| SEQ ID NO: 164 | 3′-A₁U₁G₁GIA₁-5′ |
| SEQ ID NO: 165 | 3′-C₁C₁G₁G₁U₁-5′ |
| SEQ ID NO: 166 | 3′-TAGCA-5′ |
| SEQ ID NO: 167 | 3′-TTAGCA-5′ |
| SEQ ID NO: 168 | 3′-ATGCT-5′ |
| SEQ ID NO: 169 | 3′-AATGCT-5′ |
| SEQ ID NO: 170 | 5′-TTACGA-3′ |
| SEQ ID NO: 171 | 5′-TACGA-3′ |
| SEQ ID NO: 172 | 5′-AATGCT-3′ |
| SEQ ID NO: 173 | 5′-ATGCT-3′ |
| SEQ ID NO: 174 | 3′-AGCATAGCATTGCATAAGCTTAGCA-5′ |
| SEQ ID NO: 175 | 3′-AGCATAGCATTGCATAAGCT₂T₂A₂G₂C₂A₂-5′ |
| SEQ ID NO: 176 | 3′-AGCTGAACTTGCTAAGCTTATGCATTG-5′ |
| SEQ ID NO: 177 | 3′-AGCTGAACTTGCTAAGCTTATGCATTG-5′ |
| SEQ ID NO: 178 | 3′-AGCTGAACTTGCTAAGCTTATGCATTG-5′ |
| SEQ ID NO: 179 | 3′-AGCTGAACTTGCTAAGCTTATG₂C₂A₂T₂T₂G₂-5′ |
| SEQ ID NO: 180 | 3′-AGCTGAACTTGCTAAGCTTAT-5′ |
| SEQ ID NO: 181 | 3′-AGCTGAACTTGCTAAGCT-5′ |
| SEQ ID NO: 182 | 3′-AGCTGAACTTGCT-5′ |
| SEQ ID NO: 183 | 3′-AGCTGA-5′ |
| SEQ ID NO: 184 | 3′-A₂G₂C₂T₂G₂A₂-5′ |
| SEQ ID NO: 185 | 3′-AGCTGAACTTGCTAAGCTTATGCATTG-5′ |
| SEQ ID NO: 186 | 3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| SEQ ID NO: 187 | 3′-A₂G₂C₂T₂G₂A₂A₂C₂T₂T₂G₂C₂T₂A₂A₂G₂C₂T₂T₂ATGCATTG-5′ |
| SEQ ID NO: 188 | 3′-AGCTGAACTTGCTAAGCTTAT-5′ |
| SEQ ID NO: 189 | 3′-AGCTGAACTTGCTAAGCT-5′ |
| SEQ ID NO: 190 | 3′-AGCTGAACTTGCT-5′ |
| SEQ ID NO: 191 | 3′-A₂G₂C₂T₂G₂A₂A₂C₂T₂T₂G₂C₂T₂-5′ |
| SEQ ID NO: 192 | 3′-AGCTGA-5′ |
| SEQ ID NO: 193 | 5′-TCGACTTGAACGATTCGAATACGTAAC-3′ |
| SEQ ID NO: 194 | 5′-TCGACT-3′ |
| SEQ ID NO: 195 | 3′-A₂G₂C₂A₂T₂A₂G₂C₂A₂T₂T₂G₂C₂A₂T₂A₂A₂G₂C₂T₂T₂A₂G₂C₂A₂-5′ |
| SEQ ID NO: 196 | 3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| SEQ ID NO: 197 | 3′-A₂G₂C₂T₂G₂A₂A₂C₂T₂T₂G₂C₂T₂A₂A₂G₂C₂T₂T₂A₂T₂-5′ |
| SEQ ID NO: 198 | 5′-T₂C₂G₂A₂C₂T₂T₂G₂A₂A₂C₂G₂A₂T₂T₂C₂G₂A₂A₂T₂A₂C₂G₂T₂A₂A₂C₂-3′ |
| SEQ ID NO: 199 | 3′-A₂G₂C₂T₂G₂A₂A₂C₂T₂T₂G₂C₂T₂A₂A₂G₂C₂T₂T₂A₂T₂G₂C₂A₂T₂T₂G₂-5′ |
| SEQ ID NO: 200 | 3′-A₂G₂C₂T₂G₂A₂A₂C₂T₂T₂G₂C₂T₂A₂A₂G₂C₂T₂T₂A₂T₂G₂C₂A₂T₂-5′ |
| SEQ ID NO: 201 | 3′-A₂G₂C₂T₂G₂A₂A₂C₂T₂T₂G₂C₂T₂A₂A₂G₂C₂T₂T₂A₂T₂G₂C₂A₂-5′ |
| SEQ ID NO: 202 | 3′-A₂C₂G₂T₂G₂A₂A₂C₂T₂T₂C₂G₂T₂A₂A₂C₂G₂T₂T₂A₂T₂C₂G₂A₂T₂T₂G₂-5′ |
| SEQ ID NO: 203 | 3′-A₃G₃C₃T₃G₃A₃A₃C₃T₃T₃G₃C₃T₃A₃A₃G₃C₃T₃T₃A₃T₃G₃C₃A₃T₃T₃G₃-5′ |
| SEQ ID NO: 204 | 3′-A₃G₃C₃T₃G₃A₃A₃C₃T₃T₃G₃C₃T₃A₃A₃G₃C₃T₃-5′ |
| SEQ ID NO: 205 | 3′-AGCₘTGAACTTGCₘTAAGCₘTTATGCₘATTG-5′ |
| SEQ ID NO: 206 | 3′-AGCₘTGAACTTGCₘTAAGCₘTTAT-5′ |
| SEQ ID NO: 207 | 3′-AGCₘTGAACTTGCₘTAAGCₘT-5′ |
| SEQ ID NO: 208 | 3′-AGCₘTGAACTTGCₘT-5′ |
| SEQ ID NO: 209 | 3′-AGCₘTGA-5′ |
| SEQ ID NO: 210 | 3′-AGCTGAACTTG-5′ |
| SEQ ID NO: 211 | 3′-AGCTGAAC-5′ |
| SEQ ID NO: 212 | 3′-AGCTGA-5 |
| SEQ ID NO: 213 | 5′-C₂TG₂CT₂T-3′ |
| SEQ ID NO: 214 | 5′-C₂TG₂CT₂-3′ |
| SEQ ID NO: 215 | 5′-C₃T₃G₃C₃T₃T₃-3′ |
| SEQ ID NO: 216 | 5′-C₃T₃G₃C₃T₃-3′ |
| SEQ ID NO: 217 | 5′-T₃C₃G₃A₃C₃T₃T₃G₃A₃A₃C₃G₃A₃T₃T₃C₃G₃A₃A₃T₃A₃C₃G₃T₃A₃A₃C₃-3′ |

Cₘ = 5′-methylated C; GCAT- DNA phosphorothioate linkage; G₁/C₁/A₁/U₁-2′OME phosphorothioate linkage; A₂/T₂/C₂/G₂-DNA phosphodiester linkage; A₃/T₃/C₃/G₃-RNA phosphodiester linkage

### Pharmaceutical Composition

In certain embodiments, described herein are pharmaceutical compositions comprising one or more CSO compounds of the invention. In certain embodiments, the pharmaceutical composition comprises a pharmaceutically acceptable diluent or carrier. In certain embodiments, a pharmaceutical composition comprises a sterile saline solution and one or more CSO compounds. In certain embodiments, a pharmaceutical composition consists of a sterile saline solution and one or more CSO compounds. In certain embodiments, the sterile saline is pharmaceutical grade saline. In certain embodiments, a pharmaceutical composition comprises one or more CSO compounds and sterile water. In certain embodiments, a pharmaceutical composition consists of one CSO compound and sterile water. In certain embodiments, the sterile water is pharmaceutical grade water. In certain embodiments, a pharmaceutical composition comprises one or more CSO compounds and phosphate-buffered saline (PBS). In certain embodiments, a pharmaceutical composition consists of one or more CSO compounds and sterile PBS. In certain embodiments, the sterile PBS is pharmaceutical grade PBS.

In certain embodiments, pharmaceutical compositions comprise one or more CSO compounds and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, CSO compounds of the invention may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

### Conjugate Groups

In certain embodiments, the CSOs according to the invention optionally further comprise one or more conjugate groups. Conjugate groups consist of one or more conjugate moiety and a conjugate linker which links the one or more conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups are attached at the 3′-end of oligonucleotides. In certain embodiments, conjugate groups are attached near the 3′-end of oligonucleotides. In certain embodiments, conjugate groups are attached at the 5′-end of oligonucleotides. In certain embodiments, conjugate groups are attached near the 5′-end of oligonucleotides.

In any embodiment herein, the conjugate group comprises a GalNAc cluster comprising 1-3 GalNAc ligands.

In any embodiment herein, the conjugate linker consists of a single bond.

In any embodiment herein, the conjugate linker is cleavable.

In any embodiment herein, the conjugate linker comprises 1-3 linker-nucleosides.

In any embodiment herein, wherein the conjugate group is attached to the CSO at the 5′-end of the functional domain. In any embodiment herein, wherein the conjugate group is attached to the CSO at the 3′-end of the functional domain. In any embodiment herein, wherein the conjugate group is attached to the CSO at the 5′-end of the cyclizing domain. In any embodiment herein, wherein the conjugate group is attached to the CSO at the 3′-end of the cyclizing domain.

In certain embodiments, the CSOs are covalently attached to one or more conjugate groups. In certain embodiments, conjugate groups modify one or more properties of the CSO, including but not limited to pharmacodynamics, pharmacokinetics, stability, binding, absorption, tissue distribution, cellular distribution, cellular uptake, charge and clearance. In certain embodiments, conjugate groups impart a new property on the CSO, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycerol-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Conjugate moieties are attached to the CSO through conjugate linkers. In certain embodiments, the conjugate linker is a single chemical bond (i.e., the conjugate moiety is attached directly to the CSO through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on the CSO and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-l-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

As used herein, linker-nucleosides are not considered to be part of the CSO in general or part of the cyclizing domain or the functional domain in particular. Accordingly, the nucleotides of a linker-nucleosides are not counted toward the length of the CSO or the domains thereof and are not used in determining the percent complementarity of the oligonucleotide for the reference nucleic acid. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the CSO. For example, in certain circumstances CSOs comprising a particular conjugate moiety are better taken up by a particular cell type, but once the CSO has been taken up, it is desirable that the conjugate group be cleaved to release the unconjugated or parent CSO. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

### Use

Cyclic structured oligonucleotides could be useful for various mechanisms of action. These include antisense for mRNA, ncRNA, microRNA, lncRNA and splicing. Also, double stranded cyclic structure could be used to deliver siRNA constructs. In addition, cyclic structure could provide novel approach to deliver antisense for ADAR and CRISPR based mechanisms. Furthermore, cyclic structure could provide a novel approach to deliver antisense to disrupt structures of RNA to increase translation. Cyclic structures also provide defined structures to nucleic acids thereby allowing varying degree of interaction with PRRs and induced immune cascade.

For example, when the functional domain is an antisense oligonucleotide, CSOs according to the invention are also useful in therapeutic approaches in which inhibition of gene expression is desired. This can include, for example, inhibition of an endogenous gene (e.g., an oncogene) or an exogenous gene (e.g., a gene essential for growth and/or metabolism of a pathogen).

In embodiments, the invention provides a method for inhibiting gene expression comprising administering a cyclic structured oligonucleotide as described herein or a composition comprising the cyclic structured oligonucleotide.

In embodiments, the invention provides a method for inhibiting allele-specific gene expression comprising administering a cyclic structured oligonucleotide as described herein or a composition comprising the cyclic structured oligonucleotide.

The method according to the invention are useful for treating a subject having disease or disorder wherein inhibiting expression of a gene would be beneficial. In embodiments, the disease or disorder results from abnormal expression or product of a cellular gene.

In embodiments, the cyclic structured oligonucleotide as described herein or a composition comprising the cyclic structured oligonucleotide is administered locally.

In embodiments, the cyclic structured oligonucleotide as described herein or a composition comprising the cyclic structured oligonucleotide is administered systemically.

In embodiments, a method for modulating RNA processing comprising administering a CSO compound as described herein wherein the functional domain comprises an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof. In embodiments, processing of RNA comprises splicing.

In embodiments, the invention provides a method for selecting a first mRNA transcript in a gene comprising at least two mRNA transcripts, the method comprising administering a cyclic structured oligonucleotide as described herein or a composition comprising the cyclic structured oligonucleotide.

In embodiments, the invention provides a method of treating a disease or disorder in a subject wherein modulating RNA processing would be beneficial to treat the subject, the method comprising administering a cyclic structured oligonucleotide as described herein or a composition comprising the cyclic structured oligonucleotide.

In embodiments, the invention provides a method of inducing nonsense mediated decay of a target RNA comprising administering a cyclic structured oligonucleotide as described herein or a composition comprising the cyclic structured oligonucleotide.

In embodiments, the invention provides a method of increasing a level of mRNA encoding a protein or a functional mRNA and increasing expression of the protein or the functional mRNA comprising administering a cyclic structured oligonucleotide as described herein or a composition comprising the cyclic structured oligonucleotide.

The cyclic structured oligonucleotide of the invention may be administered alone or in combination with any other agent or therapy. Agents or therapies can be co-administered or administered concomitantly. Such agent or therapy may be useful for treating or preventing the disease or condition and does not diminish the gene expression modulation effect of the cyclic oligonucleotide according to the invention. Agent(s) useful for treating or preventing the disease or condition includes, but is not limited to, small molecules, peptides vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the cyclic oligonucleotide according to the invention can be administered in combination with other compounds (for example lipids or liposomes) to enhance the specificity or magnitude of the gene expression modulation of the cyclic oligonucleotide according to the invention.

The cyclic oligonucleotide of the invention may be administered by any suitable route, including, without limitation, parenteral, mucosal delivery, oral, sublingual, transdermal, topical, inhalation, intratumoral, intravenous, subcutaneous, intrathecal, intranasal, aerosol, intraocular, intratracheal, intrarectal, vaginal, by gene gun, dermal patch or in eye drop or mouthwash form. In any of the methods according to the invention, administration of the cyclic oligonucleotide according to the invention, alone or in combination with any other agent, can be directly to a tissue or organ such as, but not limited to, the bladder, liver, lung or kidney. In certain embodiments, administration of the cyclic oligonucleotide according to the invention, alone or in combination with any other agent, is by intramuscular administration. In certain embodiments, administration of antisense oligonucleotides according to the invention, alone or in combination with any other agent, is by mucosal administration. In certain embodiments, administration of antisense oligonucleotides according to the invention, alone or in combination with any other agent, is by oral administration. In certain embodiments, administration of antisense oligonucleotides according to the invention, alone or in combination with any other agent, is by intrarectal administration. In certain embodiments, administration of antisense oligonucleotides according to the invention, alone or in combination with any other agent, is by intrathecal administration. In certain embodiments, administration of antisense oligonucleotides according to the invention, alone or in combination with any other agent, is by intratumoral administration. In certain embodiments, administration of antisense oligonucleotides according to the invention, alone or in combination with any other agent, is by parenteral administration. In certain embodiments, administration of antisense oligonucleotides according to the invention, alone or in combination with any other agent, is by subcutaneous administration.

In embodiments, any of the cyclic structured oligonucleotides described herein can be conjugated with a moiety that provides for site specific delivery of the CSO. In embodiments, such conjugates include, but are not limited to, an antibody, a peptide, a lipid, or a small molecule.

In embodiments, any of the cyclic structured oligonucleotides described herein can be encapsulated with a moiety that provides for site specific delivery of the CSO. In embodiments, the CSO can be encapsulated in, for example, a lipid, lipid nanoparticles (LNP), or peptide macrocyclic structures.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Administration of the antisense oligonucleotides according to the invention can be carried out using known procedures using an effective amount and for periods of time effective to reduce symptoms or surrogate markers of the disease. For example, an effective amount of an antisense oligonucleotide according to the invention for treating a disease and/or disorder could be that amount necessary to alleviate or reduce the symptoms, or delay or ameliorate a tumor, cancer, or bacterial, viral or fungal infection. In the context of administering a composition that modulates gene expression, an effective amount of an antisense oligonucleotide according to the invention is an amount sufficient to achieve the desired modulation as compared to the gene expression in the absence of the antisense oligonucleotide according to the invention. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular antisense oligonucleotide without necessitating undue experimentation.

### Synthesis

CSOs described herein can be prepared by any suitable art recognized method including, but not limited to, H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles), which can be carried out manually or by an automated synthesizer. The oligonucleotides of the invention may also be modified in a number of ways without compromising their ability to hybridize to their target (see e.g., Agrawal and Gait, Advances in Nucleic Acid Therapeutics, (2019) https://doi.org/10.1039/9781788015714).

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, the singular forms “a”, “an”, and “the” include both singular and plural referents unless the context clearly dictates otherwise.

As used herein, “administering” means providing a pharmaceutical agent to an animal.

As used herein, “animal” means a human or non-human animal.

As used herein, “individual in need thereof” refers to a human or non-human animal selected for treatment or therapy that is in need of such treatment or therapy.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is an increase in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is ataxia, neuropathy, and aggregate formation. In certain embodiments, amelioration of these symptoms results in improved motor function, reduced neuropathy, or reduction in number of aggregates.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases refers to nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “linker-nucleoside” means a nucleoside that links, either directly or indirectly, the CSO of the invention to a conjugate moiety. Linker-nucleosides are located within the conjugate linker and are not considered part of the CSO compound even if they are contiguous with the CSO.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid amenable to oligomeric compounds for reducing the amount or activity of the target nucleic acid as demonstrated in the examples herein below.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, the phrase “inhibiting the expression or activity” refers to a reduction or blockade of the expression or activity relative to the expression of activity in an untreated or control sample and does not necessarily indicate a total elimination of expression or activity.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethyl. “2′-MOE” means a 2′-OCH₂CH₂OCH₃ group in place of the 2′ OH group of a ribosyl sugar moiety.

As used herein, “motif” means the pattern of unmodified and/or modified sugar moieties, nucleobases, and/or internucleoside linkages, in an oligonucleotide.

As used herein, “mRNA” means an RNA transcript that encodes a protein and includes pre-mRNA and mature mRNA unless otherwise specified.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is an example of a modified nucleobase. A universal base is a modified nucleobase that can pair with any one of the five unmodified nucleobases. Modified bases, also referred to as heterocyclic base moieties, include other nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine ([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. In certain embodiments, the modified nucleobase is a 5-methylcytosine.

Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an antisense oligonucleotide may have one or more nucleotides with a sugar modification at one position and also one or more nucleotides with a sugar modification at a different position.

Sugar modifications contemplated in an oligonucleotide include, but are not limited to, a sugar substituent group selected from: OH; F; O-, S-, or N-alkyl; O-, S-, or N-alkenyl; O-, S or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)ₓCH₃, O(CH₂)ₓONH₂, and O(CH₂)ₓON((CH₂)ₓCH₃)₂, where x and y are independently from 1 to 10.

In some embodiments, the modified sugar comprises a substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an antisense oligonucleotide, or a group for improving the pharmacodynamic properties of an antisense oligonucleotide, and other substituents having similar properties. In one embodiment, the modification includes 2′-methoxyethoxy (2′-O—CH₂CH₂OCH₃, which is also known as 2′-O-(2-methoxyethyl) or 2′-MOE) (Martin et al., 1995), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl CH₂—CH═CH₂), methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics, for example, cyclobutyl moieties, in place of the pentofuranosyl sugar. Examples of U.S. patents that disclose the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, which are herein incorporated by reference in its entirety.

Representative sugar substituent groups include groups described in U.S. Patent Application Publication 2005/0261218, which is hereby incorporated by reference. In particular embodiments, the sugar modification is a 2′-O-Me modification, a 2′ F modification, a 2′ H modification, a 2′ amino modification, a 4′ thioribose modification or a phosphorothioate modification on the carboxy group linked to the carbon at position 6′, or combinations thereof.

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al, U.S. Pat. No. 7,569,686, Swayze et al., U.S. Pat. No. 7,741,457, and Swayze et al, U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al, U.S. Pat. No. 8,278,425), 4′-CH₂—O—N(CH₃)-2′ (see, e.g., Allerson et al., U.S. Pat. No. 7,696,345 and Allerson et al, U.S. Pat. No. 8,124,745), 4′-CH₂—C(H)(CH₃)-2′ (see, e.g., Zhou, et al, J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N3, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc, 20017, 129, 8362-8379; Wengel et al., U.S. Pat. No. 7,053,207; Imanishi et al., U.S. Pat. No. 6,268,490; Imanishi et al., U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

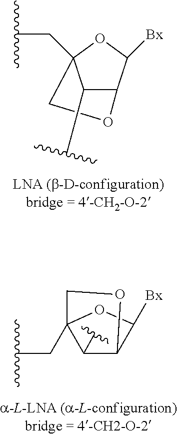

α-L-methyleneoxy (4′-CH2-0-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

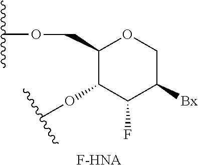

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

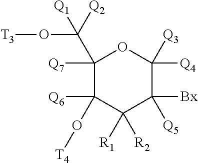

wherein, independently, for each of said modified THP nucleoside:
Bx is a nucleobase moiety;
T3 and T4 are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

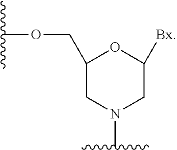

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

The nucleoside residues of the oligonucleotides of the functional or cyclizing domains can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art.

Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. In some embodiments, the synthetic antisense oligonucleotides of the invention may comprise combinations of internucleotide linkages. In some embodiments, the synthetic antisense oligonucleotides of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration.

In certain embodiments, the phosphorothioate linkages may be mixed Rp and Sp enantiomers, or they may be made stereoregular or substantially stereoregular in either Rp or Sp form. In embodiments where the linkages are mixed Rp and Sp enantiomers, the Rp and Sp forms may be at defined places within the oligonucleotide or randomly placed throughout the oligonucleotide.

As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within an animal or cells thereof. Typically, conversion of a prodrug within the animal is facilitated by the action of an enzymes (e.g., endogenous or viral enzyme) or chemicals present in cells or tissues and/or by physiologic conditions.

As used herein, “OMe” means methoxy. “2′-OMe” means a 2′-OCH₃ group in place of the 2′ OH group of a ribosyl sugar moiety.

The recitation of numerical ranges by endpoints includes all numbers and fractions subsumed within the respective ranges, as well as the recited endpoints.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

As used herein, “treat”, “treatment”, or “treating” refers to administering a compound described herein to effect an alteration or improvement of a disease, disorder, or condition.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating a disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “patient” generally refers to a mammal, such as a human. The term “mammal” is expressly intended to include warm blooded, vertebrate animals, including, without limitation, humans, non-human primates, rats, mice, cats, dogs, horses, cattle, cows, pigs, sheep and rabbits. As used herein, “individual in need thereof refers to a human or non-human animal selected for treatment or therapy that is in need of such treatment or therapy. As used herein, “inhibiting the expression or activity” refers to a reduction or blockade of the expression or activity of a RNA or protein and does not necessarily indicate a total elimination of expression or activity.

### EXAMPLES

### Synthesis of CSO Comprising an Antisense Oligonucleotide Functional Domain

Cyclic structured oligonucleotides according to the invention can be synthesized by procedures that are well known in the art, such as phosphoramidate or H-phosphonate chemistry which can be carried out manually or by an automated synthesizer. For example, the oligonucleotides of the invention may be synthesized by a linear synthesis approach.

Compounds employed herein have been synthesized using phosphoramidite chemistry. These protocols are described in detail, for example in pubs.rsc.org/en/content/chapter/bk9781788012096-00453/978-1-78801-209-6, which is incorporated herein by reference.

### Melting Temperature of CSOs of the Invention

To determine that the cyclizing domain hybridizes with the 3′-end of the oligonucleotide of the functional domain, thereby forming a cyclic structure, 2 μM samples of CSO compounds were mixed in 10 mM sodium phosphate, 100 nM sodium chloride buffer solution (pH 7.2). The samples were heated to 95° C. for 5 minutes and then allowed to slowly cool down to room temperature. The samples were stored overnight in a refrigerator before measuring the melting temperature (Tm). Cooling and heating curves from 20-90° C. are shown in Table 6.

TABLE 6

| Compound # | Tm (° C.) |
|---|---|
| 1009 | N/A |
| 1010 | N/A |
| 1101 | 43 |
| 1102 | 50 |
| 1103 | 37 |
| 1104 | 39 |
| 1105 | 45 |
| 1106 | 49 |
| 1107 | 41 |
| 1108 | 39 |
| 1109 | 31 |
| 1110 | N/A |
| 1111 | N/A |
| 1112 | N/A |
| 1124 | 64 |
| 1125 | 65 |
| 1126 | 62 |
| 1201 | 50 |
| 1202 | 44 |
| 1203 | 44 |
| 1204 | 30 |
| 1205 | N/A |
| 1206 | N/A |
| 1207 | 31 |
| 1208 | N/A |
| 1209 | N/A |
| 53 | 45 |
| 54 | 46 |
| 55 | 64 |

### Inhibition of Target RNA by CSO Comprising an Antisense Oligonucleotide Functional Domain

Cyclic structured oligonucleotides were designed targeting a PCSK9 nucleic acid and were tested for their effects on PCSK9 mRNA in vitro.

Hepa 1-6 cells were cultured in DMEM medium plus 10% FBS and 100 U/ml Pen/Strep (cells from ATCC). The cells were seeded and allowed to incubate overnight so that they are ˜70% confluent at the time of transfection—˜100,000 cells/ml/12 well plate. Cell media was changed and 900 μl was added to each well. The oligonucleotides were mixed with Lipofectamine in Opti-MEM medium, added to lipid (1:1 ratio) and incubated for 15-20 minutes. 100 μl was added to each well for an antisense concentration of 100 nM. After a treatment period of about 16 to 48 hours, cells were harvested for RNA and/or protein analysis.

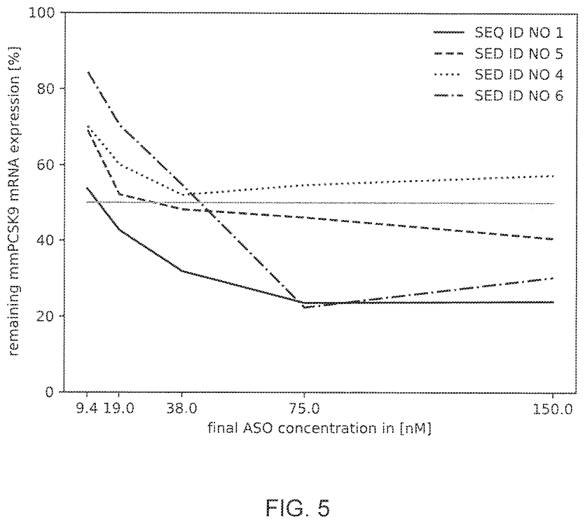

Culture supernatants were assayed for AK release cytotoxicity assay. Taqman probes for mPCSK9 and PPIB or HPRT1 (housekeeping controls) were used (probes provided by ThermoFisher). The results for each experiment are presented in FIG. 5.

Cyclic structured oligonucleotides were designed targeting a PNPLA3 nucleic acid and were tested for their effects on PNPLA3 mRNA in vitro.

Human HepG2 cells were cultured following ATCC recommended condition and media (Eagle's Minimum Essential Medium with 10% FBS). Cells were plated in PDL coated 96 well plates at 50K/well density and reverse transfected with 0.6 ul/well RNAiMax and oligonucleotide compounds of indicated concentration. 24 hrs later, cells are harvested using Cells to CT lysis reagent (ThermoFisher 4391851C). RNA was reverse transcribed to the cDNA templates using the RT reagent kit (ThermoFisher A39110). Quantitative PCR was performed using qPCR Master Mix (ThermoFisher 4444964). PNPLA3 expression level is quantified using the PNPLA3-FAM probe (ThermoFisher 4351368 Assay ID: Hs00228747) and normalized with the housekeeping gene POLR2A (ThermoFisher, 4448491, Assay ID: Hs01108291). Data are analyzed in GraphPad Prism.

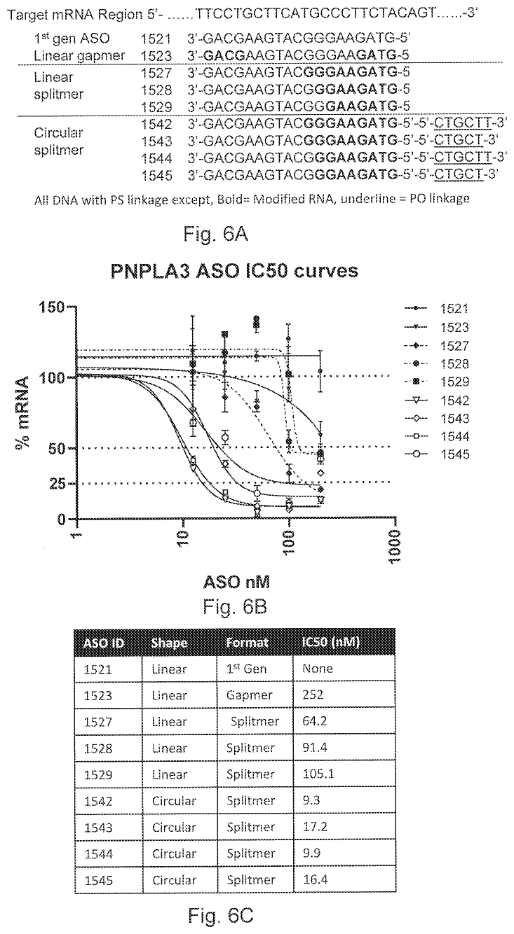

To see if circularization of linear ASO with modified RNA in a splitmer format could provide further efficacy, the level of PNPLA3 knockdown using oligonucleotides having the same base sequence in different format and chemistry arrangement were compared: linear ASO with no modified RNA ID 1521, Linear gapmer 1523, linear splitmer 1527, 1528, 1529, and circular splitmer ASO 1542, 1543, 1544, and 1545 at concentrations of 200 nM, 100 nM, 50 nM, 25 nM and 12.5 nM (FIG. 6A). As shown in FIG. 6B and FIG. 6C, 1^{st} generation format linear AS0 1521 was not active even at the higher concentration of 200 nM, the linear gapmer ASO 1523 is mildly active and can achieve about 50% knock with IC50 of 252 nM, the linear splitmer format ASOs 1527, 1528, and, 1529 can achieve better knockdown activity than the gapmer format, and all circularized splitmer ASOs (CSOs 1542, 1543, 1544, and 1545) were the most effective at reducing PNPLA3 mRNA level 24 hrs after transfection.

### Generation of Immune Response by CSO Comprising an Immunostimulatory Oligonucleotide Functional Domain

### Mouse Splenocyte Restimulation Assay

Spleens from C57BL/6J mice were mechanically dispersed into single-cell suspensions, with ammonium chloride lysis buffer (Cat #420302, BioLegend, San Diego, CA) used to remove erythrocytes. Cell viability was determined using vital dye stain and an automated cell counting system (Countess 3, Thermo Fisher, Waltham, MA). Following counting, 1×10⁵ viable mouse splenocytes were seeded into each well of a 96-well flat bottom sterile tissue culture treated plate (Cat #3596, Corning, Glendale, AZ) in RPMI 1640 (Cat #A1049101, Thermo Fisher) containing 10% Fetal Bovine Serum (Cat #F2442, MilliporeSigma, Burlington, MA).

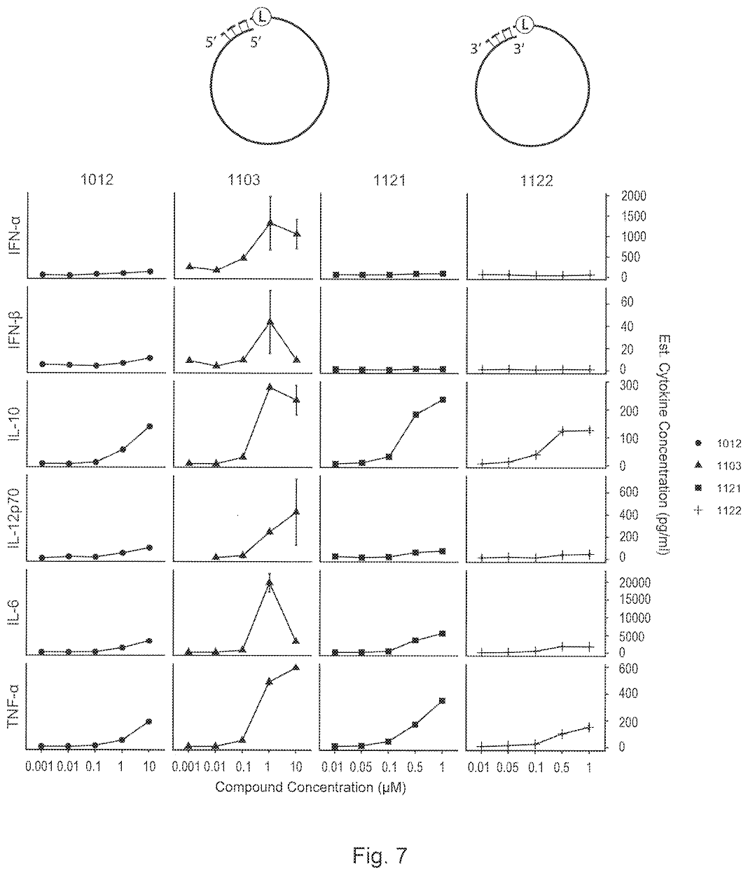

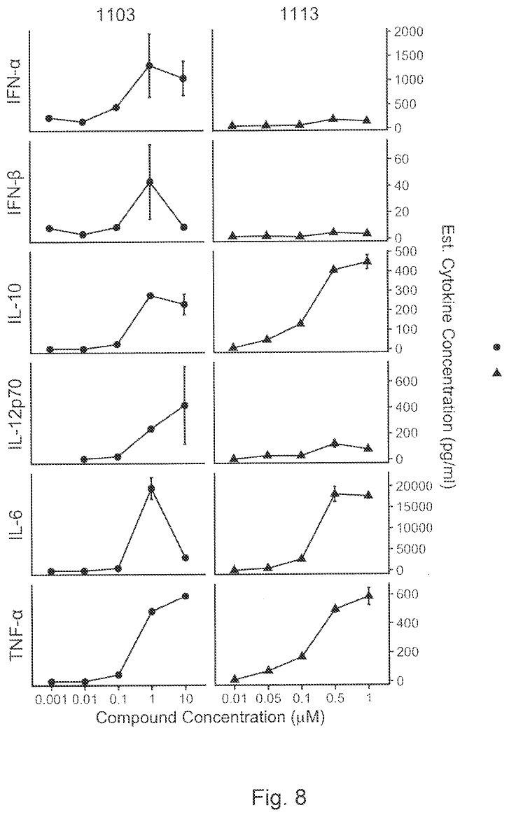

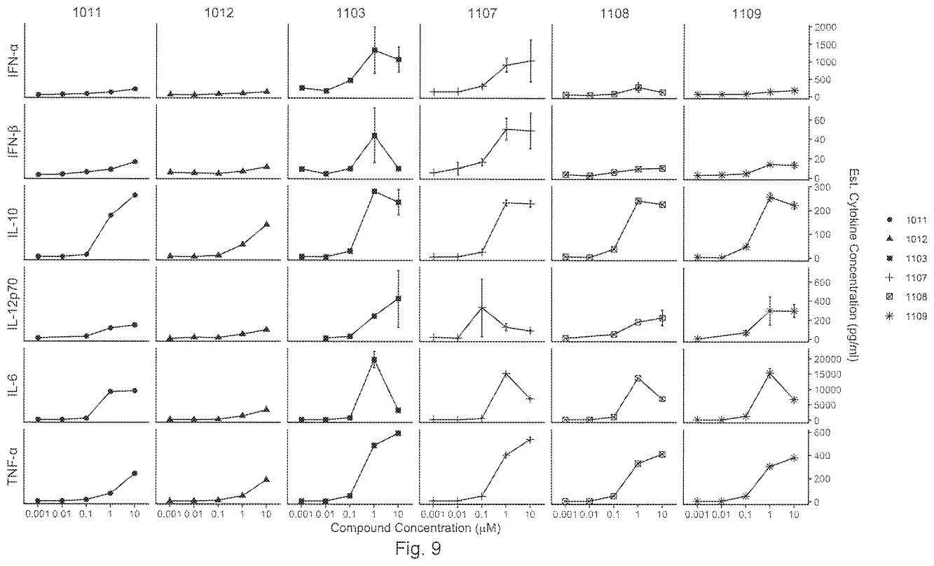

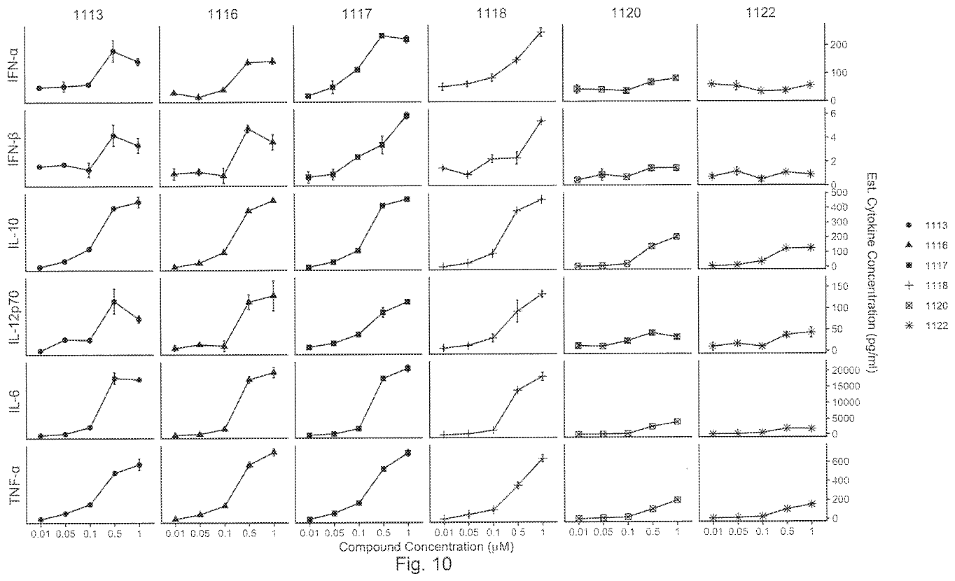

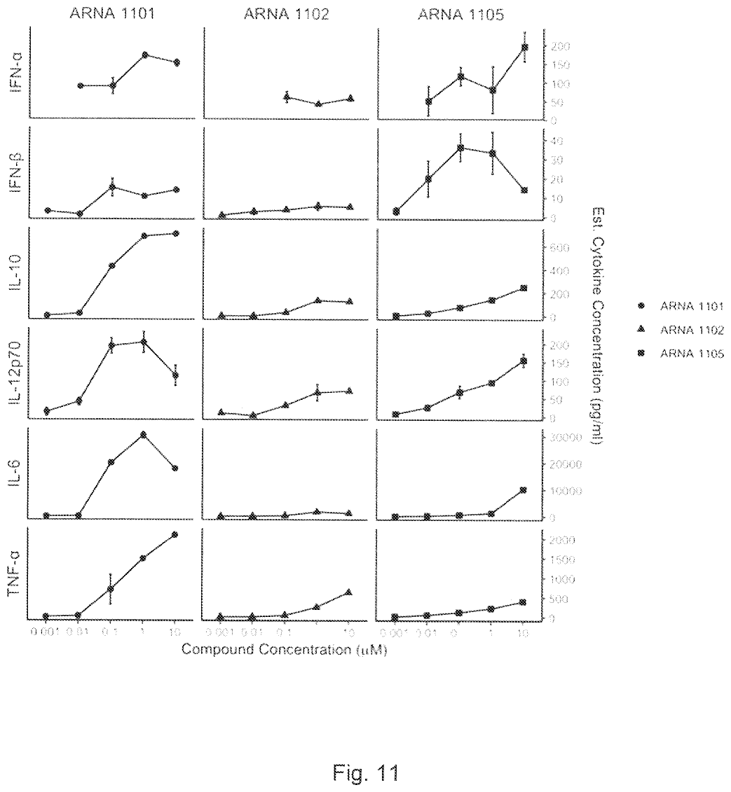

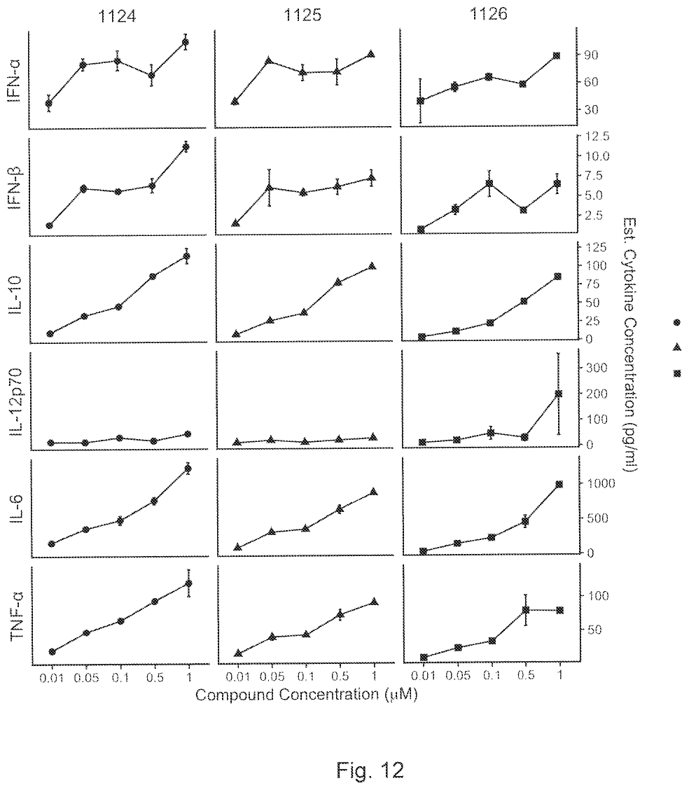

Immunostimulatory oligonucleotides (ISO) agonists for toll like receptor 9 (TLR9) and CSOs comprising TLR9 immunostimulatory oligonucleotide (ISO) functional domains were synthesized by Syngenis (Bentley, Australia) using standard methodologies and provided as lyophilized preparations, which were then reconstituted in annealing buffer (10 mM Tris, 50 mM NaCl, 1 mM EDTA, pH 7.5) using a heat block incubation step (95° C., 5 minutes). Following annealing, sample concentrations were determined using the absorbance method for oligonucleotide concentration determination using a NanoDrop spectrophotometer (Thermo Fisher). Control TLR9 agonists were obtained from Invivogen (San Diego, CA). Indicated doses of CSOs or control agonists were added to indicated wells at the indicated concentrations, and cells were stimulated for 24 hours in a 37° C., 5% CO2 incubator. Following the incubation period, cell culture supernatants were harvested and analyzed using a custom multiplex cytokine/chemokine assay kit (U-PLEX custom biomarker assay, Cat #K15069M-2, Meso Scale Diagnostics, Rockville, MD) with data collection performed on a MSD S600 bioanalyzer (Meso Scale) and raw data analysis performed through MSD Discovery Workbench (Meso Scale). Results are shown in FIGS. 7-12.

To evaluate the activity of CSOs comprising immune antagonist oligonucleotide functional domains, such CSOs were incubated alone or with a TLR9 agonist and cytokines were assessed 24 hours later.

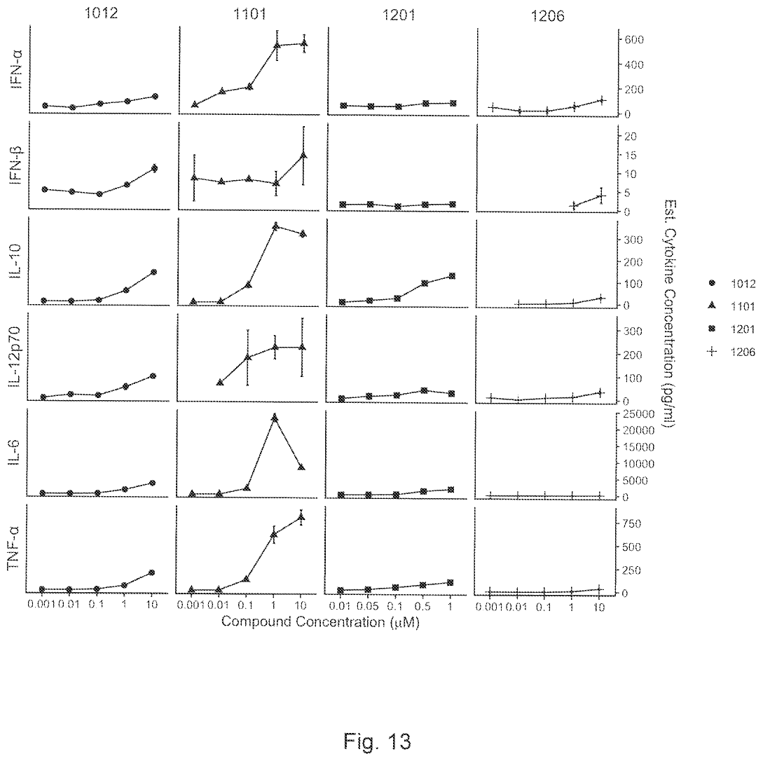

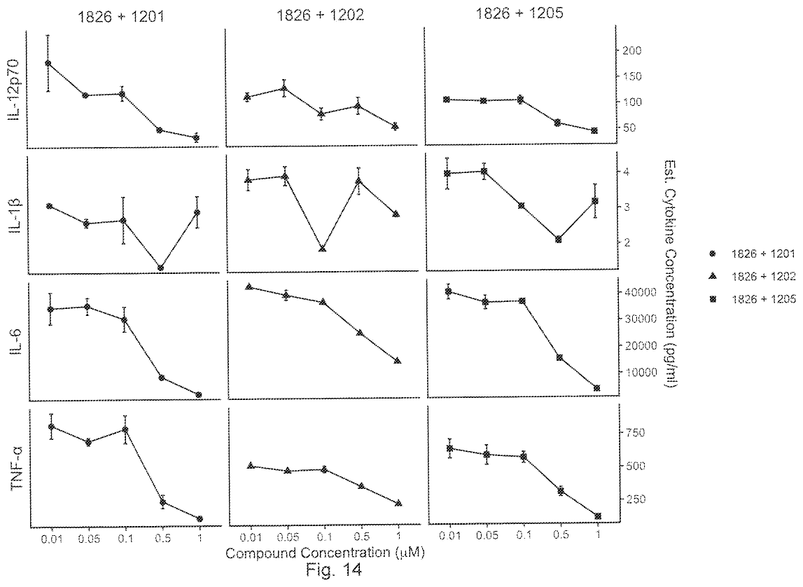

Results are shown in FIGS. 13 and 14.

While this invention has been particularly shown and described with references to preferred embodiments thereof, it will be understood by those skilled in the art that various changes in form and details may be made therein without departing from the scope of the invention encompassed by the appended claims.

1-100. (canceled)

101. A cyclic structured oligonucleotide (CSO) comprising:
a) a functional domain comprising an antisense oligonucleotide between 17 and 25 nucleotides in length, wherein the antisense oligonucleotide comprises at least 12 contiguous nucleobases complementary to an equal length portion of a target RNA sequence, and wherein the antisense oligonucleotide comprises:
i. a 3′ domain comprising 10 to 12 deoxyribonucleotides, and
ii. a 5′ domain contiguous with the 3′ domain, which comprises at least 4 modified nucleotides that are independently modified deoxyribonucleotides or modified ribonucleotides; wherein the modified nucleotides prevent RNase H cleavage in the 5′ domain;
b) a cyclizing domain comprising an oligonucleotide that is complementary to and of opposite polarity to a sequence of nucleotides within the functional domain and
c) a linker connecting the 5′ ends of the functional domain and the cyclizing domain.

102. The CSO according to claim 101, wherein the linker is a direct bond.

103. The CSO according to claim 101, wherein the cyclizing domain hybridizes with the sequence of nucleotides at the 3′-end of the oligonucleotide of the functional domain, thereby forming a cyclic structure.

104. The CSO according to claim 101, wherein the 3′ domain is 12 nucleotides in length and comprises nucleotides at positions 1, 2, 3, 4, 5, 6, 7, 8, 9, 10, 11, and 12 from the 3′ end.

105. The CSO according to claim 101, wherein the 3′ domain is 11 nucleotides in length and comprises nucleotides at positions 1, 2, 3, 4, 5, 6, 7, 8, 9, 10, and 11 from the 3′ end.

106. The CSO according to claim 101, wherein the 3′ domain is 10 nucleotides in length and comprises nucleotides at positions 1, 2, 3, 4, 5, 6, 7, 8, 9, and 10 from the 3′ end.

107. The CSO according to claim 101, wherein the 5′ domain comprises a modified internucleotide linkage or a modified nucleotide comprising a modification to a sugar a heterocyclic base, or a combination thereof.

108. The CSO according to claim 101, wherein the at least 4 modified nucleotides of the 5′ domain are modified ribonucleotides.

109. The CSO according to claim 108, wherein the modified ribonucleotides comprise 2′-substituted ribonucleotides.

110. The CSO according to claim 109, wherein the 2′-substituted ribonucleotides comprise 2′-OMe ribonucleotides or 2′MOE ribonucleotides.

111. The CSO according to claim 101, wherein each nucleotide of the 5′ domain is independently a modified deoxyribonucleotide or modified ribonucleotide.

112. The CSO according to claim 111, wherein the nucleotides of the 5′ domain are modified ribonucleotides.

113. The CSO according to claim 112, wherein the modified ribonucleotides comprise 2′-substituted ribonucleotides.

114. The CSO according to claim 113, wherein the 2′-substituted ribonucleotides comprise 2′-OMe ribonucleotides or 2′MOE ribonucleotides.

115. The CSO according to claim 101, wherein one or more conjugate groups are attached to the CSO.

116. The CSO according to claim 115, wherein the conjugate group is a GalNac or an antibody.

117. A pharmaceutical composition comprising a CSO according to claim 101 and a pharmaceutically acceptable carrier.

118. The pharmaceutical composition according to claim 117, further comprising one or more agents selected from a small molecule, a peptide, a vaccine, an antigen, an antibody, a cytotoxic agent, a kinase inhibitor, an allergen, an antibiotic, an siRNA molecule, an antisense oligonucleotide, a TLR antagonist, a chemotherapeutic agent, a targeted therapeutic agent, an activated cell, a protein, a gene therapy vector, a peptide vaccine, a protein vaccine, a DNA vaccine, an adjuvant, and a co-stimulatory molecule, or combinations thereof.

119. A method for inhibiting gene expression comprising administering a cyclic structured oligonucleotide (CSO) according to claim 101.

120. A method of treating a disease or disorder in a subject in need thereof, the method comprising administering a cyclic structured oligonucleotide (CSO) according to claim 101.
